SEQUENCE LISTING (10 sequences)

SEQ ID NO: 1 - protein, 5 aa, Artificial sequence
  synthetic
IKVAV

SEQ ID NO: 2 - protein, 15 aa, Artificial sequence
  synthetic
YRSRKYSSWYVALKR

SEQ ID NO: 3 - protein, 4 aa, Artificial sequence
  synthetic
VVAA

SEQ ID NO: 4 - protein, 4 aa, Artificial sequence
  synthetic
AAGG

SEQ ID NO: 5 - protein, 4 aa, Artificial sequence
  synthetic
AVGG

SEQ ID NO: 6 - protein, 4 aa, Artificial sequence
  synthetic
VAGG

SEQ ID NO: 7 - protein, 4 aa, Artificial sequence
  synthetic
GGGG

SEQ ID NO: 8 - protein, 4 aa, Artificial sequence
  synthetic
VAAA

SEQ ID NO: 9 - protein, 4 aa, Artificial sequence
  synthetic
AVAA

SEQ ID NO: 10 - protein, 4 aa, Artificial sequence
  synthetic
AAAA

(19) United States
(12) Patent Application Publication
(10) Pub. No.: US 2024/0325548 A1
(43) Pub. Date: Oct. 3, 2024

(54) DYNAMIC BIOACTIVE SCAFFOLDS AND THERAPEUTIC USES THEREOF AFTER CNS INJURY

(71) Applicant: Northwestern University, Evanston, IL (US)

(72) Inventors: Zaida Alvarez Pinto, Evanston, IL (US); Samuel Isaac Stupp, Evanston, IL (US)

(21) Appl. No.: 18/278,068

(22) PCT Filed: Feb. 23, 2022
(86) PCT No.: PCT/US2022/017490

§ 371 Date: Aug. 21, 2023

Related U.S. Application Data
(60) Provisional application No. 63/152,498, filed on Feb. 23, 2021.

(51) Int. Cl. A61K 47/64; A61K 38/18; A61P 25/28

(52) U.S. Cl. CPC A61K 47/64; A61K 38/18; A61K 38/1825; A61K 38/185; A61K 38/1866; A61P 25/28

(57) ABSTRACT

Provided herein are peptide amphiphiles (PAs), supramolecular assemblies comprising PAs, compositions comprising PAs, and methods of use thereof. In some embodiments, provided herein are supramolecular assemblies comprising an IKVAV PA and a growth factor mimetic PA. In some embodiments, the PAs, compositions, and supramolecular assemblies described herein are used in methods of treating nervous system injury.

### STATEMENT REGARDING RELATED APPLICATIONS

This application is a national phase application under 35 U.S.C. § 371 of PCT International Application No.: PCT/US2022/017490, filed on Feb. 23, 2022, which claims priority to U.S. Provisional Patent Application No. 63/152,498, filed Feb. 23, 2021, the entire contents of which are incorporated herein by reference for all purposes.

### STATEMENT REGARDING FEDERAL FUNDING

This invention was made with government support under agreement number FA8650-15-2-5518 awarded by the Air Force Research Laboratory, grant number ROINS104219 awarded by the National Institute on Aging, and grant numbers R21NS107761 and R21NS107761-01A1 awarded by the National Institute on Neurological Disorders and Stroke. The government has certain rights in the invention.

### SEQUENCE LISTING

The text of the computer readable sequence listing filed herewith, titled “NWEST_39312_252_SequenceListing_Corrected_ST25”, created Jan. 4, 2024, having a file size of 6,432 bytes, is hereby incorporated by reference in its entirety.

### TECHNICAL FIELD

Provided herein are peptide amphiphiles (PAs), supramolecular assemblies comprising PAs, compositions comprising PAs, and methods of use thereof. In some embodiments, provided herein are supramolecular assemblies comprising an IKVAV PA and a growth factor mimetic PA. In some embodiments, the PAs, compositions, and supramolecular assemblies described herein are used in methods of treating nervous system injury.

### BACKGROUND

The development of therapies to avoid permanent paralysis in humans after traumatic injuries remains a major challenge given the inability of damaged axons to regenerate in the adult central nervous system (CNS). An emerging signaling strategy is to use nanostructures that target specific cells to deliver a therapeutic cargo, or materials functioning as bioactive scaffolds in the extracellular space. However, success is limited by lack of understanding of how to optimize the design of such nanostructures to enable high bioactivity of the delivered cargo, resulting in a lack of highly effective therapeutics to date.

### SUMMARY

In some aspects, provided herein are supramolecular assemblies. In some embodiments, provided herein is a supramolecular assembly comprising at least two peptide amphiphiles. In some embodiments, the at least two peptide amphiphiles comprise at least one IKVAV peptide amphiphile comprising a hydrophobic segment, a structural peptide segment, a charged peptide segment, and a bioactive peptide comprising the amino acid sequence IKVAV (SEQ ID NO: 1); and at least one growth factor mimetic peptide amphiphile. In some embodiments, the at least one IKVAV peptide amphiphile comprises a fluorescence anisotropy value of less than 0.3. In some embodiments, the at least one IKVAV peptide amphiphile comprises a proton relaxation rate (¹H—R₂) of less than 4 s¹.

In some embodiments, the hydrophobic segment comprises an 8-24 carbon alkyl chain (C₈₋₂₄). In some embodiments, the hydrophobic segment comprises a 16 carbon alkyl chain (C₁₆). In some embodiments, the structural peptide segment comprises A₂G₂(SEQ ID NO: 4). In some embodiments, the charged peptide segment comprises E₂, E₃, or E₄(SEQ ID NO: 11). In some embodiments, the bioactive peptide is attached to the charged peptide segment by a linker. In some embodiments, the linker is a single glycine (G) residue. In some embodiments, the IKVAV peptide amphiphile comprises C16-A2G2E4GIKVAV (SEQ ID NO: 12).

In some embodiments, the at least one growth factor mimetic peptide amphiphile comprises a hydrophobic segment comprising an 8-24 carbon alkyl chain (C₈₋₂₄), a structural peptide segment comprising V₂A2, A₂G2, a charged peptide segment comprising E₂, E₃, or E₄, and a growth factor mimetic peptide sequence. In some embodiments, the growth factor mimetic sequence is a vascular endothelial growth factor (VEGF) mimetic sequence, a fibroblast growth factor 2 (FGF-2) mimetic sequence, a Glial cell-derived neurotrophic factor (GDNF) mimetic sequence, a brain-derived neurotrophic factor (BDNF) mimetic sequence, or a Netrin-1 mimetic sequence. In some embodiments, the growth factor mimetic sequence is an FGF-2 mimetic sequence. For example, in some embodiments the FGF-2 mimetic sequence comprises YRSRKYSSWYVALKR (SEQ ID NO: 2). In some embodiments, the growth factor mimetic peptide is attached to the charged peptide segment by a linker. In some embodiments, the linker is a single glycine (G) residue.

In some embodiments, the at least one growth factor mimetic peptide amphiphile comprises C₁₆-V₂A₂E₄GYRSRKYSSWYVALKR (SEQ ID NO: SEQ ID NO: 13) or C₁₆. A₂G₂E₄GYRSRKYSSWYVALKR (SEQ ID NO: 14). In some embodiments, the at least one IKVAV peptide amphiphile comprises C₁₆-A₂G₂E₄GIKVAV (SEQ ID NO: 12) and the at least one growth factor mimetic peptide amphiphile comprises C₁₆V₂A₂E₄GYRSRKYSSWYVALKR (SEQ ID NO: 13) or C₁-A₂G₂E₄GYRSRKYSSWYVALKR (SEQ ID NO: 14). In some embodiments, the at least one IKVAV peptide amphiphile comprises C₁₆-A₂G₂E₄GIKVAV (SEQ ID NO: 12) and the at least one growth factor mimetic peptide amphiphile comprises C₁₆V₂A₂E₄GYRSRKYSSWYVALKR (SEQ ID NO: 13).

In some aspects, provided herein are compositions. In some embodiments, provided herein is a composition comprising a supramolecular assembly as described herein. In some embodiments, provided herein is a composition comprising at least one IKVAV peptide amphiphile comprising a hydrophobic segment, a structural peptide segment, a charged peptide segment, and a bioactive peptide comprising the amino acid sequence IKVAV; and at least one growth factor mimetic peptide amphiphile. In some embodiments, the at least one IKVAV peptide amphiphile and the at least one growth factor mimetic peptide amphiphile interact to form a supramolecular assembly within the composition.

In some embodiments, the at least one IKVAV peptide amphiphile comprises a fluorescence anisotropy value of less than 0.3. In some embodiments, the at least one IKVAV peptide amphiphile comprises a proton relaxation rate (¹H—R₂) of less than 4 s⁻¹. In some embodiments, the hydrophobic segment comprises an 8-24 carbon alkyl chain (C₈₋₂₄). For example, in some embodiments the hydrophobic segment comprises a 16 carbon alkyl chain (C₁₆). In some embodiments, the structural peptide segment comprises A₂G₂. In some embodiments, the charged peptide segment comprises E₂, E₃, or E₄. In some embodiments, the bioactive peptide is attached to the charged peptide segment by a linker. In some embodiments, the linker is a single glycine (G) residue. In some embodiments, the IKVAV peptide amphiphile comprises C₁₆-A₂G₂E₄GIKVAV (SEQ ID NO: 12).

In some embodiments, the at least one growth factor mimetic peptide amphiphile comprises a hydrophobic segment comprising an 8-24 carbon alkyl chain (C₈₋₂₄), a structural peptide segment comprising V₂A₂(SEQ ID NO: 3) or A₂G₂(SEQ ID NO: 4), a charged peptide segment comprising E₂, E₃, or E₄ (SEQ ID NO: 11), and a growth factor mimetic peptide sequence. In some embodiments, the growth factor mimetic sequence is a vascular endothelial growth factor (VEGF) mimetic sequence, a fibroblast growth factor 2 (FGF-2) mimetic sequence, a Glial cell-derived neurotrophic factor (GDNF) mimetic sequence, a brain-derived neurotrophic factor (BDNF) mimetic sequence, or a Netrin-1 mimetic sequence. In some embodiments, the growth factor mimetic sequence is an FGF-2 mimetic sequence. In some embodiments, the FGF-2 mimetic sequence comprises YRSRKYSSWYVALKR (SEQ ID NO: 2). In some embodiments, the growth factor mimetic peptide is attached to the charged peptide segment by a linker. In some embodiments, the linker is a single glycine (G) residue.

In some embodiments, the at least one growth factor mimetic peptide amphiphile comprises C₁₆-V₂A₂E₄GYRSRKYSSWYVALKR (SEQ ID NO: 13) or C₁₆-A₂G₂E₄GYRSRKYSSWYVALKR (SEQ ID NO: 14). In some embodiments, the at least one IKVAV peptide amphiphile comprises C₁₆-A₂G₂E₄GIKVAV (SEQ ID NO: 12) and the at least one growth factor mimetic peptide amphiphile comprises C₁₆-V₂A₂E₄GYRSRKYSSWYVALKR (SEQ ID NO: 13) or C₁₆-A₂G₂E₄GYRSRKYSSWYVALKR (SEQ ID NO: 14). In some embodiments, the at least one IKVAV peptide amphiphile comprises C₁-A₂G₂E₄GIKVAV (SEQ ID NO: 12) and the at least one growth factor mimetic peptide amphiphile comprises C₁₆-V₂A₂E₄GYRSRKYSSWYVALKR (SEQ ID NO: 13).

The compositions described herein may be used in a method of treating a nervous system injury in a subject. In some embodiments, the nervous system injury is a central nervous system injury. For example, in some embodiments the central nervous system injury is a spinal cord injury.

In some aspects, provided herein are methods of treating a nervous system in jury in a subject. In some embodiments, provided herein is a method of treating a nervous system injury in a subject, comprising providing to the subject a composition as described herein. In some embodiments, the nervous system injury is a central nervous system injury. In some embodiments, the central nervous system injury is a spinal cord injury.

Other aspects and embodiments of the disclosure will become apparent in view of the appended materials and figures.

### BRIEF DESCRIPTION OF THE DRAWINGS

FIG. 1A-1E shows a library of investigated IKVAV PA molecules. (A) Specific chemical structures of IKVAV PA molecules used and molecular graphics representation of a supramolecular nanofiber displaying the IKVAV bioactive signal. (B) Cryo-TEM micrographs of IKVAV PAs in the library and their corresponding color-coded representation of RMSF values for single IKVAV PA filaments. (C) Bar graphs of the average RMSF values of the different IKVAV PA molecules (error bars correspond to 3 independent simulations; *P<0.05, **P<0.01, ***P<0.001, one-way ANOVA with Bonferroni). (D) SAXS patterns and (E) WAXS profiles of the different IKVAV PA nanofibers (the scattering intensities were offset vertically for clarity; the Bragg peak corresponding to the β-sheet spacing around 1.35 Å is framed in a gray box). Scale bars: 200 nm.

FIG. 2A-2J shows the effect of supramolecular motion on hNPCs signaling in vitro. (A) Molecular graphics representation of an IKVAV PA nanofiber indicating the chemical structure and location of DPH used as a probe in fluorescence depolarization measurements (top); bar graph of fluorescence anisotropy of IKVAV PA solutions (error bars correspond to 3 independent experiments; n.s. no significant, ***P<0.0001, one-way ANOVA with Bonferroni). (B) Chemical structure of the IKVAV peptide sequence highlighting the K residue probed by NMR (top); bar graphs of the K relaxation time for the different IKVAV PAs investigated (error bars correspond to 3 runs per condition; ***P<0.0001 vs IKVAV PA1, ^{#}P<0.05, ^{###}P<0.0001 vs IKVAV PA2 and ⁺P<0.05, ⁺⁺⁺P<0.0001 vs IKVAV PA5, one-way ANOVA with Bonferroni). (C) Differentiation conditions used for hNPCs. (D) Representative micrographs of hNPCs treated with IKVAV PA1, PA2, PA4 and PA5; NESTIN-stem cells (red), ITGB1-receptor (green), and DAPI-nuclei (blue). (E) WB results of ITGB1, p-FAK, FAK, ILK, and TUJ-1 in hNPCs treated with laminin and the various IKVAV PAs. (F) Representative confocal micrographs of hNPCs treated with IKVAV PA1, PA2, PA4 and PA5; NESTIN-stem cells (red), SOX-2-stem cells (green), TUJ-1-neurons (white), and DAPI-nuclei (blue). (G, H) Bar graphs of the percentage of SOX-2⁺ and NESTIN⁺-stem cells (G) and TUJ-1⁺ neuronal cells (H) treated with the various IKVAV PAs (error bars correspond to 3 independent differentiations; **P<0.01, ***P<0.001 vs IKVAV PA2 and ^{##}P<0.01, ^{###}P<0.001 vs IKVAV PA5, one-way ANOVA with Bonferroni). (I) Fluorescence anisotropy (left) and K residue relaxation times (right) obtained for IKVAV PA2 nanofibers in the absence (No Ca²⁺) or presence (Ca²⁺) of calcium ions (***P<0.001, student's t-test). (J) WB results of ITGB1, p-FAK, FAK, and ILK in hNPCs treated with IKVAV PA2 in the absence (−) or presence (+) of Ca²⁺. Scale bars: (D) 10 mm, (F) 100 mm.

FIG. 3A-3L shows that two chemically different PA scaffolds with two identical bioactive sequences reveal differences in growth of corticospinal axons after SCI. (A) Chemical structures of the two PA molecules used. (B) Molecular graphics representation of a supramolecular nanofiber displaying two bioactive signals (top); cryo-TEM micrographs of IKVAV PA2 co-assembled with FGF2 PAs (FGF2 PA1 and FGF2 PA2) (bottom). (C) Storage modulus of IKVAV PA2 (green) and their respective co-assemblies with FGF2 PAs (FGF2 PA1, red and FGF2 PA2, blue). (D) Fluorescent micrographs of spinal cords (green) injected with IKVAV PA2+FGF2 PA1 (red) covalently labeled with Alexa 647. (E) Dot plot of PA scaffold volume as a function of time after implantation. (F) Schematic illustration showing the site of BDA and PA injections (left); fluorescent micrographs of the brain cortex (top, right); NeuN-neurons (green), BDA-labelled neurons (red) and DAPI-nuclei (blue) and transverse spinal cord section stained for GFAP-astrocytes (green), BDA-labelled descending axons (red) and DAPI-nuclei (blue) (bottom, right). (G) Fluorescent micrographs of longitudinal spinal cord sections in sham, IKVAV PA2+FGF2 PA1, and IKVAV PA2+FGF2 PA2 groups; GFAP-astrocytes (green), BDA-labelled axons (red) and DAPI-nuclei (blue); vertical white dashed lines indicate the proximal border (PB), the distal border (DB), and the central part of the lesion (LC). (H) Representative magnified images for those in G. (I) Schematic lesion site and vertical lines used to count the number of axons crossing at each location indicated (top); plot of the number of crossing axons (bottom) (error bars correspond to 6 animals per group; *P<0.05, **P<0.01, ***P<0.001 vs sham and ^{#}P<0.05, ^{##}P<0.01, ^{###}P<0.001 vs IKVAV PA2 and IKVAV PA2+FGF2 PA2 groups, repeated measures of two-way ANOVA with Bonferroni). (J) WB results (left) and dot plot of the normalized values for GAP43 and MBP protein in sham, IKVAV PA2, IKVAV PA2+FGF2 PA1, and IKVAV PA2+FGF2 PA2 (right) (**P<0.01, ***P<0.001 vs sham and ###P<0.001 vs IKVAV PA2+FGF2 PA1 group, one-way ANOVA with Bonferroni). (K) Representative 3D fluorescent micrographs of BDA-labelled axon regrowth (red) and myelin basic protein (MBP, green) (left) and laminin (white) (right). (L) WB results (left) and dot plot of the normalized values for laminin and fibronectin expression in conditions described in J (right) (***P<0.001 vs sham and ^{#}P<0.05, #P<0.01, ^{###}P<0.001 vs IKVAV PA2+FGF2 PA1 group, one-way ANOVA with Bonferroni). Data points in E correspond to 3 animals per group and to 4 animals per group in J and L. Scale bars: (D, G) 1500 m, (F) 25 m (top) and 200 m (bottom), (H) 100 m, and (K) 2 m.

FIGS. 4A-4E show that two chemically different PA scaffolds with two identical bioactive sequences reveal differences in angiogenesis. (A) Fluorescent micrographs of transverse spinal cord sections in uninjured, IKVAV PA2+FGF2 PA1, IKVAV PA2+FGF2 PA2 and sham groups; GFAP-astrocytes (green), DiI-labelled blood vessels (red), and DAPI-nuclei (blue). (B) Dot plots of the vascular area fraction, perfused vascular length, and number of branches in the transverse sections of groups in A (*P<0.05, ***P<0.0001 vs sham and ^{##}P<0.001, ###P<0.0001 vs IKVAV PA2+FGF2 PA1 group, one-way ANOVA with Bonferroni). (C) Fluorescent images of BrdU⁺/CD31⁺ cells in the center of the lesion in animals injected with IKVAV PA2+FGF2 PA1 and IKVAV PA2+FGF2 PA2; CD31-blood vessels (green), BrdU-newly generated cells (red), and DAPI-nuclei (blue). (D) Dot plot of the number of BrdU⁺/CD31⁺ cells per mm² in groups treated with IKVAV PA2 alone, IKVAV PA2+FGF2 PA1, IKVAV PA2+FGF2 PA2, and saline (sham) (*P<0.05, ***P<0.001 vs sham and ###P<0.0001 vs IKVAV PA2+FGF2 PA1 group, one-way ANOVA with Bonferroni). (E) WB results (left) and dot plot of the normalized values for CD31 protein (right) (**P<0.001, ***P<0.0001 vs sham and ###P<0.001 vs IKVAV PA2+FGF2 PA1 group one-way ANOVA with Bonferroni). Data points in B and D correspond to 6 animals per group and to 4 animals per group in E. Scale bars: (A) 200 m, (C) 25 m.

FIG. 5A-5D show that two chemically different PA scaffolds with two identical bioactive sequences reveal differences in neuronal survival and functional recovery. (A) Fluorescent micrographs of transverse spinal cord sections corresponding to uninjured, IKVAV PA2+FGF2 PA1, IKVAV PA2+FGF2 PA2 and sham groups; NeuN-neurons (green), DiI-labelled blood vessels (red) and DAPI-nuclei (blue), dashed lines indicate the grey matter (horn). (B) High-magnification images of the ventral horn area for slices in A (left); NeuN-neurons (green), DiI-labelled blood vessels (red), and DAPI-nuclei (blue); ChAT-motor neurons (green), DiI-labelled blood vessels (red), and DAPI-nuclei (blue) (right). (C) Dot plots showing the number of NeuN⁺ (left) and ChAT⁺ (right) cells per transverse section (data points correspond to a total of 48 sections; 8 sections per animal and 6 animals per group; **P<0.01, ***P<0.001 vs sham and ###P<0.001 vs IKVAV PA2+FGF2 PA1 group, one-way ANOVA with Bonferroni). (D) Experimental timeline of in vivo experiments (top) and Basso Mouse Scale (BMS) for locomotion (bottom) (error bars correspond to 38 animals per group; **P<0.001, ***P<0.0001 all PA groups vs sham and ###P<0.0001 vs IKVAV PA2+FGF2 PA2 and IKVAV PA2 groups by repeated measures of two-way ANOVA with Bonferroni). Scale bars: (A) 200 m, (B) 25 m.

FIG. 6A-6J show data validating cell signaling differences in vitro between two PA scaffolds exhibiting different supramolecular motion. (A) Confocal micrographs of HUVECs treated with IKVAV PA2+FGF2 PA1 and IKVAV PA2+FGF2 PA2; ACTIN-cytoskeleton (red), DAPI-nuclei (blue). (B) Bar graph of the number of branches in HUVECs treated with laminin+FGF-2, IKVAV PA2 alone, IKVAV PA2+FGF2 PA1, and IKVAV PA2+FGF2 PA2. (C) WB results (left) and bar graphs of the normalized values for active FGFR1 (p-FGFR1) vs total FGFR1 (FGFR1) and active ERK1/2 (p-ERK1/2) using the conditions in B (right). (D) Confocal micrographs of hNPCs on coatings of IKVAV PA2+FGF2 PA1 and IKVAV PA2+FGF2 PA2; EDU-proliferative marker (red), SOX-2-neural stem cell marker (green), and DAPI-nuclei (blue). (E) Bar graph of the percentage of EDU+/SOX-2⁺ cells on the various coatings. (F) WB results (left) and bar graphs of the normalized values for active FGFR1 (p-FGFR1) vs total FGFR1 (FGFR1) and b1 INTEGRIN (ITGB1) (right). (G) ¹H-NMR spin-spin relaxation time of the aromatic protons in Y and W amino acids in the FGF2 mimetic signal at 6.81 ppm (solid lines are single linear best fits). (H) Bar graph of the aromatic relaxation times measured in G (error bars correspond to 3 runs per condition; *P<0.05 student's t-test). (I) Fluorescence anisotropy of FGF2 PAs chemically modified with Cy3 dye (error bars correspond to 3 independent experiments; ***P<0.001 student's t-test). (J) Color-coded representation of RMSF values in clusters of FGF2 PAs (left) and the corresponding bar graph (right) (IKVAV PA2 molecules are shown in transparent grey, ions and water molecules are removed for clarity, and the simulation box is shown in blue) (error bars correspond to 5 independent simulations; **P<0.01 Student's T-test). Error bars in B and E correspond to 3 independent experiments and C and F correspond to 4 independent experiments per condition; ***P<0.0001 vs Laminin+FGF2 and ###P<0.0001 vs IKVAV PA2+FGF2 PA1, one-way ANOVA with Bonferroni. Scale bars: (A) 200 μm, (D) 100 μm.

FIG. 7 shows the chemical structure (left) and mass spectra (right) of IKVAV PAs.

FIG. 8 shows plots representing the root mean square deviation (RMSD) vs. time of IKVAV PAs.

FIG. 9 shows bar graphs of the normalized values for ITGB1, p-FAK/FAK, ILK, and TUJ-1 in hNPCs cultured on ornithine coatings and treated with laminin and the library of IKVAV PAs (error bars correspond to 3 independent differentiations; **P<0.01, ***P<0.001 vs. IKVAV PA2 and ^{#}P<0.05, ^{##}P<0.01, ^{##}P<0.001 vs. IKVAV PA5, one-way ANOVA with Bonferroni).

FIG. 10A-10C shows the effect of calcium on supramolecular motion and in vitro cell signaling. (A) Anisotropy of IKVAV PA2 and IKVAV PA5 solutions in the absence (No Ca²⁺) or presence (Ca²⁺) of calcium (error bars correspond to 3 independent experiments; **P<0.01, ***P<0.001, Student's T-test). (B) Representative fluorescent micrographs of hNPCs cultured on the conditions mentioned in A. (C) WB results (left) and corresponding bar graph representing the normalized protein levels for of ITGB1, p-FAK/FAK, ILK, and TUJ-1 (right) in hNPCs treated with IKVAV PA2 or IKVAV PA5 in the absence (−) or presence (+) of calcium (Ca²⁺) (calcium alone (+) was used as a control; error bars correspond to 3 independent experiments per condition; **P<0.01, ***P<0.001, Student's T-test). Scale bars: 10 mm.

FIG. 11A-11C show cryo-TEM images and storage modulus of IKVAV PA2 co-assembled with FGF2 PAs at different molar ratios. Cryo-TEM micrographs (left) and storage modulus (right) of IKVAV PA2 co-assembled with FGF2 PAs (PA1 or PA2) at molar ratios of (A) 95:5, (B) 90:10, and (C) 80:20 (data points correspond to 3 replicas per condition; n.s. indicates not significant, *P<0.05, Student's two-tailed T-test). Scale bars: 200 nm.

FIG. 12A-12F show the characterization of co-assembled IKVAV PA2+FGF2 PAs systems. (A) SAXS scattering pattern, (B) WAXS profile, and (C) FT-IR of IKVAV PA2 (green), IKVAV PA2+FGF2 PA1 (red) and IKVAV PA2+FGF2 PA2 (blue). (D) High-magnification negative TEM images of IKVAV PA2 (green), IKVAV PA2+FGF2 PA1 (red) and IKVAV PA2+FGF2 PA2 (blue). (E) Fiber width of conditions mentioned in D (error bars correspond to 50 fibers measured per condition; **P<0.01 vs. IKVAV PA2 and ^{#}P<0.05 vs. IKVAV PA2+FGF2 PA1 by one-way ANOVA with Bonferroni). (F) Optical density (O.D.) plot at 600 nm of IKVAV PA2, FGF2 PA1, FGF2 PA2 and its co-assemblies at different percentages (left) and photographs of FGF2 PA (orange) and co-assembled with IKVAV PA2 (red) (right) (error bars correspond to 3 replicas per condition; ***P<0.0001 vs. its co-assembled samples one-way ANOVA with Bonferroni). Scale bars: 100 nm.

FIG. 13A-13E show cleared spinal cords injected with dual signal co-assemblies. (A) Chemical structures and (B) mass spectra of the Alexa Fluor® 647-labeled IKVAV PA2. (C) and Cryo-TEM image of Alexa Fluor® 647-labeled IKVAV PA2. (D) Photographs of mouse spinal cords injected with PA before (non-cleared) and after (Cleared) clearing the tissue. (E) Full micrograph reconstructions of mouse spinal cords (green) injected with IKVAV PA2+FGF2 PA1 or IKVAV PA2+FGF2 PA2 (both in red) where IKVAV PA2 contains 1% of Alexa Fluor® 647-labeled. Scale bar: (C) 100 nm and (E) 1000 m.

FIG. 14A-14D show the effect of IKVAV PAs on CST axon regrowth. (A) Fluorescent micrographs of longitudinal spinal cord sections in Backbone PA, IKVAV PA1, IKVAV PA2, and IKVAV PA4 groups; GFAP-astrocytes (green), BDA-labelled axons (red) and DAPI-nuclei (blue); vertical white dashed lines indicate the proximal border (PB), the distal border (DB), and the central part of the lesion (LC). (B) Representative magnified images for those in A. (C) Schematic lesion site and vertical lines used to count the number of axons crossing at each location indicated (top); plot of the number of crossing axons (bottom) (error bars correspond to 6 animals per group; *P<0.05, **P<0.01, ***P<0.001 vs sham, +P<0.05, ++P<0.01 vs. IKVAV PA1 and #P<0.05 vs. IKVAV PA4 groups, repeated measures of two-way ANOVA with Bonferroni). (D) WB results (bottom) and corresponding bar graph representing the normalized protein levels of GFAP for sham, Backbone PA, IKVAV PA1, IKVAV PA2, and IKVAV PA4 (top) (data points correspond to 4 animals per condition; ***P<0.001 vs. sham, #P<0.05 vs. Backbone PA, one-way ANOVA with Bonferroni). Scale bars: (A) 1500 m and (B) 100 m.

FIG. 15A-15F show the effect of IKVAV PA2 co-assembled with FGF2 PAs on axonal regrowth and glial scar formation. (A) Fluorescence images of longitudinal spinal cord sections of animals injected with saline solution (sham), IKVAV PA2+FGF2 PA1, IKVAV PA2+FGF2 PA2, and IKVAV PA2 alone; BDA-labelled descending axons (red) and DAPI-nuclei (blue); vertical white dashed lines indicate the proximal border (PB) and the distal border (DB). (B) Detailed images of BDA-labeled axons (red) in the center of the lesion for the conditions mentioned in A; vertical white dashed lines indicate the central part of the lesion (LC). (C) Representative images of longitudinal spinal cord sections stained for GFAP-astrocytes (green) and DAPI-nuclei (blue) within the lesion border in conditions mentioned in A. (D) WB results (bottom) and corresponding bar graph representing the normalized protein levels for GFAP using the conditions in A (top) (data points correspond to 4 animals per condition; ***P<0.001 vs. sham, one-way ANOVA with Bonferroni). (E) Representative 3D fluorescence micrographs taken in the center of the lesion of BDA-labeled axon (red), GFAP-astrocytes (green), and DAPI-nuclei (blue). (F) 3D micrograph reconstruction of BDA-labeled axon regrowth covered with myelin basic protein (MBP, green) (top) and along laminin (white) (bottom) in IKVAV PA2 group. Scale bars: (A, C) 100 m, (B) 50 m, and (E, F) 2 m.

FIG. 16A-16E show the effect of IKVAV PA2 co-assembled with FGF2 PAs on serotoninergic neuronal regrowth. (A) Fluorescent micrographs of longitudinal spinal cord sections in sham, IKVAV PA2+FGF2 PA1, IKVAV PA2+FGF2 PA2 and IKVAV PA2 groups; Laminin-ECM (green), 5HT-serotoninergic neurons (red) and DAPI-nuclei (blue). (B, C) Representative magnified images of the (B) proximal border (PB) and (C) distal border (DB) for those in A; vertical white dashed lines indicate the PB and DB. (D) Schematic lesion site and vertical lines used to count the number of 5HT axons crossing at each location indicated (top); plot of the number of crossing axons (bottom) (error bars correspond to 6 animals per group; *P<0.05, **P<0.01 vs. sham and ^{#}P<0.05 vs. IKVAV PA2 and IKVAV PA2+FGF2 PA2 groups, repeated measures of two-way ANOVA with Bonferroni). (E) Representative magnified image of the lesion center (LC) in IKVAV PA2+FGF2 PA1. Scale bars: (A) 1500 m and (B,C) 100 m (E) 50 m.

FIG. 17A-17E shows footprint analysis of animals injected with dual signal co-assemblies. (A) Representative photographs of mouse hindlimb positioning when walking after 3 months post-injury in Sham, IKVAV PA2+FGF2 PA1, IKVAV PA2+FGF2 PA2 groups. (B) Bar graph representing the impact force used to create the lesion in the spinal cords of animals treated with saline solution (Sham), IKVAV PA2+FGF2 PA1, IKVAV PA2+FGF2 PA2, IKVAV PA2 (data points show 38 animals analyzed; n.s. indicates not significant). (C) Representative footprints of animals injected with the various conditions plotted in B. (D, E) Bar graphs representing (D) stride length in mm and (E) stride width in mm of animals injected with the various conditions mentioned in B (data points correspond to 38 animals per condition; ***P<0.0001 vs. sham and ^{#}P<0.0001 vs. IKVAV PA2+FGF2 PA1 group, one-way ANOVA with Bonferroni).

FIG. 18 shows ¹H-NMR of PA co-assemblies. ¹H-NMR spectra of the aromatic protons in Y and W amino acids in the FGF2 mimetic sequence in IKVAV PA2+FGF2 PA1 and IKVAV PA2+FGF2 PA2 systems recorded at 600 MHz.

### DEFINITIONS

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies or protocols herein described, as these may vary in accordance with routine experimentation and optimization. It is also to be understood that the terminology used in the description is for the purpose of describing the particular versions or embodiments only, and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a”, “an” and “the” include plural reference unless the context clearly dictates otherwise. Thus, for example, reference to “a peptide amphiphile” is a reference to one or more peptide amphiphiles and equivalents thereof known to those skilled in the art, and so forth.

As used herein, the term “comprise” and linguistic variations thereof denote the presence of recited feature(s), element(s), method step(s), etc. without the exclusion of the presence of additional feature(s), element(s), method step(s), etc. Conversely, the term “consisting of” and linguistic variations thereof, denotes the presence of recited feature(s), element(s), method step(s), etc. and excludes any unrecited feature(s), element(s), method step(s), etc., except for ordinarily-associated impurities. The phrase “consisting essentially of” denotes the recited feature(s), element(s), method step(s), etc. and any additional feature(s), element(s), method step(s), etc. that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

The term “amino acid” refers to natural amino acids, unnatural amino acids, and amino acid analogs, all in their D and L stereoisomers, unless otherwise indicated, if their structures allow such stereoisomeric forms.

Natural amino acids include alanine (Ala or A), arginine (Arg or R), asparagine (Asn or N), aspartic acid (Asp or D), cysteine (Cys or C), glutamine (Gln or Q), glutamic acid (Glu or E), glycine (Gly or G), histidine (His or H), isoleucine (Ile or I), leucine (Leu or L), Lysine (Lys or K), methionine (Met or M), phenylalanine (Phe or F), proline (Pro or P), serine (Ser or S), threonine (Thr or T), tryptophan (Trp or W), tyrosine (Tyr or Y) and valine (Val or V).

Unnatural amino acids include, but are not limited to, azetidinecarboxylic acid, 2-aminoadipic acid, 3-aminoadipic acid, beta-alanine, naphthylalanine (“naph”), aminopropionic acid, 2-aminobutyric acid, 4-aminobutyric acid, 6-aminocaproic acid, 2-aminoheptanoic acid, 2-aminoisobutyric acid, 3-aminoisbutyric acid, 2-aminopimelic acid, tertiary-butylglycine (“tBuG”), 2,4-diaminoisobutyric acid, desmosine, 2,2′-diaminopimelic acid, 2,3-diaminopropionic acid, N-ethylglycine, N-ethylasparagine, homoproline (“hPro” or “homoP”), hydroxylysine, allo-hydroxylysine, 3-hydroxyproline (“3Hyp”), 4-hydroxyproline (“4Hyp”), isodesmosine, allo-isoleucine, N-methylalanine (“MeAla” or “Nime”), N-alkylglycine (“NAG”) including N-methylglycine, N-methylisoleucine, N-alkylpentylglycine (“NAPG”) including N-methylpentylglycine. N-methylvaline, naphthylalanine, norvaline (“Norval”), norleucine (“Norleu”), octylglycine (“OctG”), ornithine (“Orn”), pentylglycine (“pG” or “PGly”), pipecolic acid, thioproline (“ThioP” or “tPro”), homoLysine (“hLys”), and homoArginine (“hArg”).

The term “amino acid analog” refers to a natural or unnatural amino acid where one or more of the C-terminal carboxy group, the N-terminal amino group and side-chain bioactive group has been chemically blocked, reversibly or irreversibly, or otherwise modified to another bioactive group. For example, aspartic acid-(beta-methyl ester) is an amino acid analog of aspartic acid; N-ethylglycine is an amino acid analog of glycine; or alanine carboxamide is an amino acid analog of alanine. Other amino acid analogs include methionine sulfoxide, methionine sulfone, S-(carboxymethyl)-cysteine, S-(carboxymethyl)-cysteine sulfoxide and S-(carboxymethyl)-cysteine sulfone.

As used herein, the term “peptide” refers an oligomer to short polymer of amino acids linked together by peptide bonds. In contrast to other amino acid polymers (e.g., proteins, polypeptides, etc.), peptides are of about 50 amino acids or less in length. A peptide may comprise natural amino acids, non-natural amino acids, amino acid analogs, and/or modified amino acids. A peptide may be a subsequence of naturally occurring protein or a non-natural (artificial) sequence.

As used herein, the term “artificial” refers to compositions and systems that are designed or prepared by man, and are not naturally occurring. For example, an artificial peptide, peptoid, or nucleic acid is one comprising a non-natural sequence (e.g., a peptide without 100% identity with a naturally-occurring protein or a fragment thereof).

As used herein, a “conservative” amino acid substitution refers to the substitution of an amino acid in a peptide or polypeptide with another amino acid having similar chemical properties, such as size or charge. For purposes of the present disclosure, each of the following eight groups contains amino acids that are conservative substitutions for one another:
1) Alanine (A) and Glycine (G);
2) Aspartic acid (D) and Glutamic acid (E);
3) Asparagine (N) and Glutamine (Q);
4) Arginine (R) and Lysine (K);
5) Isoleucine (I), Leucine (L), Methionine (M), and Valine (V);
6) Phenylalanine (F), Tyrosine (Y), and Tryptophan (W);
7) Serine (S) and Threonine (T); and
8) Cysteine (C) and Methionine (M).

Naturally occurring residues may be divided into classes based on common side chain properties, for example: polar positive (or basic) (histidine (H), lysine (K), and arginine (R)); polar negative (or acidic) (aspartic acid (D), glutamic acid (E)); polar neutral (serine (S), threonine (T), asparagine (N), glutamine (Q)); non-polar aliphatic (alanine (A), valine (V), leucine (L), isoleucine (I), methionine (M)); non-polar aromatic (phenylalanine (F), tyrosine (Y), tryptophan (W)); proline and glycine; and cysteine. As used herein, a “semi-conservative” amino acid substitution refers to the substitution of an amino acid in a peptide or polypeptide with another amino acid within the same class.

In some embodiments, unless otherwise specified, a conservative or semi-conservative amino acid substitution may also encompass non-naturally occurring amino acid residues that have similar chemical properties to the natural residue. These non-natural residues are typically incorporated by chemical peptide synthesis rather than by synthesis in biological systems. These include, but are not limited to, peptidomimetics and other reversed or inverted forms of amino acid moieties. Embodiments herein may, in some embodiments, be limited to natural amino acids, non-natural amino acids, and/or amino acid analogs.

Non-conservative substitutions may involve the exchange of a member of one class for a member from another class.

As used herein, the term “sequence identity” refers to the degree of which two polymer sequences (e.g., peptide, polypeptide, nucleic acid, etc.) have the same sequential composition of monomer subunits. The term “sequence similarity” refers to the degree with which two polymer sequences (e.g., peptide, polypeptide, nucleic acid, etc.) differ only by conservative and/or semi-conservative amino acid substitutions. The “percent sequence identity” (or “percent sequence similarity”) is calculated by: (1) comparing two optimally aligned sequences over a window of comparison (e.g., the length of the longer sequence, the length of the shorter sequence, a specified window, etc.), (2) determining the number of positions containing identical (or similar) monomers (e.g., same amino acids occurs in both sequences, similar amino acid occurs in both sequences) to yield the number of matched positions, (3) dividing the number of matched positions by the total number of positions in the comparison window (e.g., the length of the longer sequence, the length of the shorter sequence, a specified window), and (4) multiplying the result by 100 to yield the percent sequence identity or percent sequence similarity. For example, if peptides A and B are both 20 amino acids in length and have identical amino acids at all but 1 position, then peptide A and peptide B have 95% sequence identity. If the amino acids at the non-identical position shared the same biophysical characteristics (e.g., both were acidic), then peptide A and peptide B would have 100% sequence similarity. As another example, if peptide C is 20 amino acids in length and peptide D is 15 amino acids in length, and 14 out of 15 amino acids in peptide D are identical to those of a portion of peptide C, then peptides C and D have 70% sequence identity, but peptide D has 93.3% sequence identity to an optimal comparison window of peptide C. For the purpose of calculating “percent sequence identity” (or “percent sequence similarity”) herein, any gaps in aligned sequences are treated as mismatches at that position.

Any polypeptides described herein as having a particular percent sequence identity or similarity (e.g., at least 70%) with a reference sequence ID number, may also be expressed as having a maximum number of substitutions (or terminal deletions) with respect to that reference sequence. For example, a sequence having at least Y % sequence identity (e.g., 90%) with SEQ ID NO:Z (e.g., 100 amino acids) may have up to X substitutions (e.g., 10) relative to SEQ ID NO:Z, and may therefore also be expressed as “having X (e.g., 10) or fewer substitutions relative to SEQ ID NO:Z.”

As used herein, the term “nanofiber” refers to an elongated or threadlike filament (e.g., having a significantly greater length dimension that width or diameter) with a diameter typically less than 100 nanometers.

As used herein, the term “supramolecular” (e.g., “supramolecular complex,” “supramolecular interactions,” “supramolecular fiber,” “supramolecular polymer,” etc.) refers to the non-covalent interactions between molecules (e.g., polymers, macromolecules, etc.) and the multicomponent assemblies, complexes, systems, and/or fibers that form as a result.

As used herein, the terms “self-assemble” and “self-assembly” refer to formation of a discrete, non-random, aggregate structure from component parts; said assembly occurring spontaneously through random movements of the components (e.g. molecules) due only to the inherent chemical or structural properties and attractive forces of those components.

As used herein, the term “peptide amphiphile” refers to a molecule that, at a minimum, includes a hydrophobic segment, a structural peptide segment and/or charged peptide segment (often both). In some embodiments, a peptide amphiphile includes a bioactive peptide (e.g. an IKVAV peptide, a growth factor mimetic peptide). In some embodiments, a peptide amphiphile includes a linker (e.g. G). The peptide amphiphile may express a net charge at physiological pH, either a net positive or negative net charge, or may be zwitterionic (i.e., carrying both positive and negative charges). Certain peptide amphiphiles consist of or comprise: (1) a hydrophobic, non-peptide segment (e.g., comprising an acyl group of six or more carbons), (2) a structural peptide segment; (3) a charged peptide segment, and (4) a bioactive peptide segment (e.g. IKVAV peptide, growth factor mimetic peptide).

As used herein and in the appended claims, the term “lipophilic moiety” or “hydrophobic moiety” refers to the moiety (e.g., an acyl, ether, sulfonamide, or phosphodiester moiety) disposed on one terminus (e.g., C-terminus, N-terminus) of the peptide amphiphile, and may be herein and elsewhere referred to as the lipophilic or hydrophobic segment or component. The hydrophobic segment should be of a sufficient length to provide amphiphilic behavior and aggregate (or nanosphere or nanofiber) formation in water or another polar solvent system. Accordingly, in the context of the embodiments described herein, the hydrophobic component preferably comprises a single, linear acyl chain of the formula: Cₙ₋₁H₂ₙ₋₁C(O)— where n=2-25. In some embodiments, a linear acyl chain is the lipophilic group (saturated or unsaturated carbons), palmitic acid. However, other lipophilic groups may be used in place of the acyl chain such as steroids, phospholipids and fluorocarbons.

As used interchangeably herein, the terms “structural peptide” or “structural peptide segment” refer to a portion of a peptide amphiphile, typically disposed between the hydrophobic segment and the charged peptide segment. The structural peptide is generally composed of three to ten amino acid residues with non-polar, uncharged side chains (e.g., His (H), Val (V), Ile (I), Leu (L), Ala (A), Phe (F)) selected for their propensity to form hydrogen bonds or other stabilizing interactions (e.g., hydrophobic interactions, van der Waals' interactions, etc.) with structural peptide segments of adjacent structural peptide segments. In some embodiments, the structural peptide segment has a propensity to form α-helix and/or β-sheet secondary structures. In some embodiments, assemblies of peptide amphiphiles having structural peptide segments display linear or 2D structure when examined by microscopy and/or α-helix and/or β-sheet character when examined by circular dichroism (CD).

In some embodiments, the structural peptide segment has a low propensity to for α-helix and/or β-sheet secondary structures. For example, in some embodiments the structural peptide segment has a total propensity for forming β-sheet conformations of 4 or less. In some embodiments, assemblies of peptide amphiphiles having structural peptide segments with a total propensity for forming β-sheet conformations of 4 or less display a less ordered character (e.g. less ordered secondary structure, such as less rigid β-sheet conformations). Such PAs may be advantageous as they possess a relatively high degree of internal motion, enabling the formation of dynamic supramolecular assemblies. In some embodiments, nanofibers of peptide amphiphiles having structural peptide segments with a total propensity for forming β-sheet conformations of 4 or less (e.g. A₂G₂(SEQ ID NO: 4), GGGG (SEQ ID NO: 7)) display a propensity to form random coil structures.

As used herein, the term “beta (β)-sheet-forming peptide segment” refers to a structural peptide segment that has a propensity to display β-sheet-like character (e.g., when analyzed by CD). In some embodiments, amino acids in a beta (β)-sheet-forming peptide segment are selected for their propensity to form a beta-sheet secondary structure. Examples of suitable amino acid residues selected from the twenty naturally occurring amino acids include Met (M), Val (V), Ile (I), Cys (C), Tyr (Y), Phe (F), Gln (Q), Leu (L), Thr (T), Ala (A), and Gly (G) (listed in order of their propensity to form beta sheets). However, non-naturally occurring amino acids of similar beta-sheet forming propensity may also be used. Peptide segments capable of interacting to form beta sheets and/or with a propensity to form beta sheets are understood (See, e.g., Mayo et al. Protein Science (1996), 5:1301-1315; herein incorporated by reference in its entirety).

As used herein, the term “charged peptide segment” refers to a portion of a peptide amphiphile that is rich (e.g., >50%, >75%, etc.) in charged amino acid residues, or amino acid residue that have a net positive or negative charge under physiologic conditions. A charged peptide segment may be acidic (e.g., negatively charged), basic (e.g., positively charged), or zwitterionic (e.g., having both acidic and basic residues).

As used herein, the terms “carboxy-rich peptide segment,” “acidic peptide segment,” and “negatively-charged peptide segment” refer to a peptide sequence of a peptide amphiphile that comprises one or more amino acid residues that have side chains displaying carboxylic acid side chains (e.g., Glu (E), Asp (D), or non-natural amino acids). A carboxy-rich peptide segment may optionally contain one or more additional (e.g., non-acidic) amino acid residues. Non-natural amino acid residues, or peptidomimetics with acidic side chains could be used, as will be evident to one ordinarily skilled in the art. There may be from about 2 to about 7 amino acids, and or about 3 or 4 amino acids in this segment.

As used herein, the terms “amino-rich peptide segment”, “basic peptide segment,” and “positively-charged peptide segment” refer to a peptide sequence of a peptide amphiphile that comprises one or more amino acid residues that have side chains displaying positively-charged acid side chains (e.g., Arg (R), Lys (K), His (H), or non-natural amino acids, or peptidomimetics). A basic peptide segment may optionally contain one or more additional (e.g., non-basic) amino acid residues. Non-natural amino acid residues with basic side chains could be used, as will be evident to one ordinarily skilled in the art. There may be from about 2 to about 7 amino acids, and or about 3 or 4 amino acids in this segment.

As used herein, the term “bioactive peptide” refers to amino acid sequences that mediate the action of sequences, molecules, or supramolecular complexes associated therewith. Peptide amphiphiles and structures (e.g., nanofibers) bearing bioactive peptides (e.g., an IKVAV peptide) exhibit the functionality of the bioactive peptide. In some embodiments, a “bioactive peptide” comprising the bioactive amino acid sequence IKVAV (SEQ ID NO: 1) is referred to herein as an “IKVAV peptide amphiphile” or an “IKVAV PA”. In some embodiments, a “bioactive peptide” is a peptide comprising a growth factor mimetic peptide sequence. A bioactive peptide comprising a growth factor mimetic peptide sequence is referred to herein as a “growth factor mimetic peptide amphiphile” or a “growth factor mimetic PA”.

As used herein, the term “biocompatible” refers to materials and agents that are not toxic to cells or organisms. In some embodiments, a substance is considered to be “biocompatible” if its addition to cells in vitro results in less than or equal to approximately 10% cell death, usually less than 5%, more usually less than 1%.

As used herein, “biodegradable” as used to describe the polymers, hydrogels, and/or wound dressings herein refers to compositions degraded or otherwise “broken down” under exposure to physiological conditions. In some embodiments, a biodegradable substance is a broken down by cellular machinery, enzymatic degradation, chemical processes, hydrolysis, etc. In some embodiments, a wound dressing or coating comprises hydrolyzable ester linkages that provide the biodegradability.

As used herein, the phrase “physiological conditions” relates to the range of chemical (e.g., pH, ionic strength) and biochemical (e.g., enzyme concentrations) conditions likely to be encountered in the intracellular and extracellular fluids of tissues. For most tissues, the physiological pH ranges from about 7.0 to 7.4.

As used herein, the terms “treat,” “treatment,” and “treating” refer to reducing the amount or severity of a particular condition, disease state (e.g., CNS injury), or symptoms thereof, in a subject presently experiencing or afflicted with the condition or disease state. The terms do not necessarily indicate complete treatment (e.g., total elimination of the condition, disease, or symptoms thereof). “Treatment,” encompasses any administration or application of a therapeutic or technique for a disease (e.g., in a mammal, including a human), and includes inhibiting the disease, arresting its development, relieving the disease, causing regression, or restoring or repairing a lost, missing, or defective function; or stimulating an inefficient process.

As used herein, the terms “prevent,” “prevention,” and preventing” refer to reducing the likelihood of a particular condition or disease state (e.g., CNS injury) from occurring in a subject not presently experiencing or afflicted with the condition or disease state. The terms do not necessarily indicate complete or absolute prevention. For example “preventing CNS injury” refers to reducing the likelihood of CNS injury occurring in a subject not presently experiencing or diagnosed with a CNS injury. In order to “prevent CNS injury” a composition or method need only reduce the likelihood of CNS injury, not completely block any possibility thereof. “Prevention,” encompasses any administration or application of a therapeutic or technique to reduce the likelihood of a disease developing (e.g., in a mammal, including a human). Such a likelihood may be assessed for a population or for an individual.

As used herein, the terms “co-administration” and “co-administering” refer to the administration of at least two agent(s) or therapies to a subject (e.g., a supramolecular assembly described herein and one or more therapeutic agents). In some embodiments, the co-administration of two or more agents or therapies is concurrent. In other embodiments, a first agent/therapy is administered prior to a second agent/therapy. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful (e.g., toxic) agent(s), and/or when co-administration of two or more agents results in sensitization of a subject to beneficial effects of one of the agents via co-administration of the other agent.

### DETAILED DESCRIPTION

Provided herein are peptide amphiphiles (PAs), compositions comprising PAs, supramolecular assemblies comprising PAs, and methods of use thereof.

In some aspects, provided herein are peptide amphiphiles. In some embodiments, the peptide amphiphile molecules and compositions of the embodiments described herein are synthesized using preparatory techniques well-known to those skilled in the art, preferably, by standard solid-phase peptide synthesis, with the addition of a fatty acid in place of a standard amino acid at the N-terminus (or C-terminus) of the peptide, in order to create the lipophilic segment (although in some embodiments, alignment of nanofibers is performed via techniques not previously disclosed or used in the art (e.g., extrusion through a mesh screen). Synthesis typically starts from the C-terminus, to which amino acids are sequentially added using either a Rink amide resin (resulting in an —NH2 group at the C-terminus of the peptide after cleavage from the resin), or a Wang resin (resulting in an —OH group at the C-terminus). Accordingly, some embodiments described herein encompass peptide amphiphiles having a C-terminal moiety that may be selected from the group consisting of —H, —OH, —COOH, —CONH2, and —NH2.

In some embodiments, peptide amphiphiles comprise a hydrophobic segment (i.e. a hydrophobic tail) linked to a peptide. In some embodiments, the peptide comprises a structural peptide segment. In some embodiments, the structural peptide segment is a hydrogen-bond-forming segment, or beta-sheet-forming segment. In some embodiments, the structural peptide segment has the propensity to form random coil structures (e.g. a total propensity for forming (3-sheet conformations of 4 or less). In some embodiments, the structural peptide segment has a low propensity to form ordered secondary structures and therefore possesses a relatively high level of internal motion.

In some embodiments, the peptide comprises a charged segment (e.g., acidic segment, basic segment, zwitterionic segment, etc.). In some embodiments, the peptide further comprises linker or spacer segments for adding solubility, flexibility, distance between segments, etc. In some embodiments, peptide amphiphiles comprise a spacer segment (e.g., peptide and/or non-peptide spacer) at the opposite terminus of the peptide from the hydrophobic segment. In some embodiments, the spacer segment comprises peptide and/or non-peptide elements. In some embodiments, the spacer segment comprises one or more bioactive groups (e.g., alkene, alkyne, azide, thiol, etc.). In some embodiments, various segments may be connected by linker segments (e.g., peptide (e.g., GG) or non-peptide (e.g., alkyl, OEG, PEG, etc.) linkers).

The lipophilic or hydrophobic segment is typically incorporated at the N- or C-terminus of the peptide after the last amino acid coupling, and is composed of a fatty acid or other acid that is linked to the N- or C-terminal amino acid through an acyl bond. In aqueous solutions, PA molecules self-assemble (e.g., into cylindrical micelles (a.k.a., nanofibers)) to bury the lipophilic segment in their core and display the bioactive peptide on the surface. In some embodiments, the structural peptide undergoes intermolecular hydrogen bonding to form beta sheets that orient parallel to the long axis of the micelle. In some embodiments, the structural peptide displays weak intermolecular hydrogen bonding, resulting in a less rigid beta-sheet conformation within the nanofibers.

In some embodiments, a hydrophobic (e.g., hydrocarbon and/or alkyl/alkenyl/alkynyl tail, or steroid such as cholesterol) segment of sufficient length (e.g., 2 carbons, 3 carbons, 4 carbons, 5 carbons, 6 carbons, 7 carbons, 8 carbons, 9 carbons, 10 carbons, 11 carbons, 12 carbons, 13 carbons, 14 carbons, 15 carbons, 16 carbons, 17 carbons, 18 carbons, 19 carbons, 20 carbons, 21 carbons, 22 carbons, 23 carbons, 24 carbons, 25 carbons, 26 carbons, 27 carbons, 28 carbons, 29 carbons, 30 carbons or more, or any ranges there between) is covalently coupled to peptide segment (e.g., a peptide a structural peptide segment and a charged peptide segment) to yield a peptide amphiphile molecule. In some embodiments, a plurality of such PAs will self-assemble in water (or aqueous solution) into a nanostructure (e.g., nanofiber). In various embodiments, the relative lengths of the peptide segment and hydrophobic segment result in differing PA molecular shape and nanostructural architecture. For example, a broader peptide segment and narrower hydrophobic segment results in a generally conical molecular shape that has an effect on the assembly of PAs (See, e.g., J. N. Israelachvili Intermolecular and surface forces; 2nd ed.; Academic: London San Diego, 1992; herein incorporated by reference in its entirety). Other molecular shapes have similar effects on assembly and nanostructural architecture.

In some embodiments, to induce self-assembly of an aqueous solution of peptide amphiphiles, the pH of the solution may be changed (raised or lowered) or multivalent ions, such as calcium, or charged polymers or other macromolecules may be added to the solution.

In some embodiments, the hydrophobic segment is a non-peptide segment (e.g., alkyl/alkenyl/alkynyl group). In some embodiments, the hydrophobic segment comprises an alkyl chain (e.g., saturated) of 4-25 carbons (e.g., 4, 5, 6, 7, 8, 9, 10, 11, 12, 13, 14, 15, 16, 17, 18, 19, 20, 21, 22, 23, 24, 25), fluorinated segments, fluorinated alkyl tails, heterocyclic rings, aromatic segments, pi-conjugated segments, cycloalkyls, oligothiophenes etc. In some embodiments, the hydrophobic segment comprises an acyl/ether chain (e.g., saturated) of 2-30 carbons (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30). In some embodiments, the hydrophobic segment comprises an 8-24 carbon alkyl chain (C₈₋₂₄). In some embodiments, the hydrophobic segment comprises a 16 carbon alkyl chain (C₁₆).

In some embodiments, PAs comprise one or more peptide segments. Peptide segment may comprise natural amino acids, modified amino acids, unnatural amino acids, amino acid analogs, peptidomimetics, or combinations thereof. In some embodiments, peptide segment comprise at least 50% sequence identity or similarity (e.g., conservative or semi-conservative) to one or more of the peptide sequences described herein.

In some embodiments, peptide amphiphiles comprise a charged peptide segment. The charged segment may be acidic, basic, or zwitterionic.

In some embodiments, peptide amphiphiles comprise an acidic peptide segment. For example, in some embodiments, the acidic peptide comprises one or more (e.g., 1, 2, 3, 4, 5, 6, 7, or more) acidic residues (D and/or E) in sequence. In some embodiments, the acidic peptide segment comprises up to 7 residues in length and comprises at least 50% acidic residues. In some embodiments, an acidic peptide segment comprises (Xa)₁₋₇, wherein each Xa is independently D or E. In some embodiments, an acidic peptide segment comprises E₂₋₄. For example, in some embodiments an acidic peptide segment comprises E₂. In some embodiments, an acidic peptide segment comprises E₃. In other embodiments, an acidic peptide segment comprises E₄.

In some embodiments, peptide amphiphiles comprise a basic peptide segment. For example, in some embodiments, the acidic peptide comprises one or more (e.g., 1, 2, 3, 4, 5, 6, 7, or more) basic residues (R, H, and/or K) in sequence. In some embodiments, the basic peptide segment comprises up to 7 residues in length and comprises at least 50% basic residues. In some embodiments, an acidic peptide segment comprises (Xb)₁₋₇, wherein each Xb is independently R, H, and/or K.

In some embodiments, peptide amphiphiles comprises a structural peptide segment. In some embodiments, the structural peptide segment is a beta-sheet-forming segment. In some embodiments, the structural peptide segment displays weak hydrogen bonding and lacks secondary structure. In some embodiments, the structural peptide segment displays weak hydrogen bonding and has the propensity to form random coil structures rather than rigid beta-sheet conformations. In some embodiments, the structural peptide segment is rich in one or more of H, I, L, F, V, G, and A residues. In some embodiments, the structural peptide segment comprises an alanine- and valine-rich peptide segment (e.g., V₂A₂(SEQ ID NO: 3), V₃A₃(SEQ ID NO: 19), A₂V₂(SEQ ID NO: 31), A₃V₃(SEQ ID NO: 16), or other combinations of V and A residues, etc.). In some embodiments, the structural peptide segment comprises 4 or more consecutive A and/or V residues, or conservative or semi-conservative substitutions thereto. In some embodiments, the structural peptide segment comprises V₂A₂(SEQ ID NO: 3). In some embodiments, the structural peptide segment comprises an alanine and glycine-rich peptide segment (e.g. A₂G₂(EQ ID NO: 4), A₃G₃(SEQ ID NO: 17), or other combinations of A and G residues, etc.). In some embodiments, the structural peptide segment comprises A₂G₂(SEQ ID NO: 4). In some embodiments, the structural peptide segment comprises a glycine-rich peptide segment. For example, in some embodiments the structural peptide segment comprises G₃ or G₄ (SEQ ID NO: 7).

In some embodiments, the structural peptide segment has a total propensity for forming j-sheet conformations of 4 or less (e.g. less than 4, less than 3.9, less than 3.8, less than 3.7, less than 3.6, less than 3.5, less than 3.4, less than 3.3, less than 3.2, less than 3.1, less than 3.0, less than 2.9. less than 2.8, less than 2.7, less than 2.6, less than 2.5, less than 2.4, less than 2.3, less than 2.2, less than 2.1, less than 2.0, less than 1.9, less than 1.8, less than 1.7, less than 1.6, less than 1.5, less than 1.4, less than 1.3, less than 1.2, less than 1.1, or less than 1.)

The total propensity for forming β-sheet conformations may be calculated as the sum of the propensity for forming β-sheet conformations of each amino acid in the structural peptide segment. The propensity of each amino acid for forming β-sheet conformations and methods for calculating the same are described in, for example, Fujiwara, K., Toda, H. & Ikeguchi, M. Dependence of α-helical and β-sheet amino acid propensities on the overall protein fold type. BMC Struct Biol 12, 18 (2012), the entire contents of which are incorporated herein by reference. Exemplary values are shown in Table 1, below. For the purposes of calculating the total propensity for forming β-sheet conformations of the structural peptide segment, the value shown in the “total residues” column from table 1 for each amino acid is added together. For example, for an A2G2 structural peptide segment, the total propensity for forming β-sheet conformations is 0.75+0.75+0.67+0.67=2.84. The structural peptide segment may comprise any suitable number and combination of amino acids to achieve a total propensity for forming β-sheet conformations of 4 or less.

TABLE 1
Amino acid Propensities for β-sheet conformations

| Amino Acid | Exposed Residues | Buried Residues | Total Residues |
|---|---|---|---|
| V | 2.31 | 1.57 | 2.00 |
| I | 2.02 | 1.39 | 1.79 |
| L | 1.18 | 0.93 | 1.15 |
| M | 1.01 | 0.84 | 1.01 |
| P | 0.49 | 0.42 | 0.40 |
| A | 0.48 | 0.72 | 0.75 |
| C | 1.24 | 1.07 | 1.36 |
| F | 1.50 | 1.10 | 1.4 |
| Y | 1.71 | 1.12 | 1.37 |
| W | 1.90 | 0.91 | 1.23 |
| Q | 0.96 | 0.82 | 0.72 |
| S | 0.86 | 0.85 | .081 |
| T | 1.58 | 1.08 | 1.21 |
| N | 0.71 | 0.76 | 0.63 |
| H | 1.15 | 0.98 | 0.99 |
| D | 0.61 | 0.76 | 0.55 |
| K | 1.14 | 0.98 | 0.76 |
| E | 0.89 | 0.86 | 0.65 |
| R | 1.27 | 0.82 | 0.85 |
| G | 0/41 | 0.81 | 0.67 |

In some embodiments, a structural peptide segment having a total propensity for forming β-sheet conformations of 4 or less indicates that the amino acids within the structural peptide segment have weaker interactions with neighboring molecules. For example, the structural peptide segment may display weak hydrogen-bonding abilities. Accordingly, such structural peptide segments and the peptide amphiphiles comprising the same may create more dynamic supramolecular assemblies. For example, an A₂G₂ structural peptide segment may display random coil structures rather than rigid beta-sheet conformations.

In some embodiments, a bioactive peptide amphiphile (e.g. an IKVAV peptide amphiphile) comprises a relatively low fluorescent anisotropy value. Anisotropy is calculated using the following equation:

$$A = \frac{{F}_{‖} - {gF}_{⊥}}{{F}_{‖} + 2 {gF}_{⊥}}$$

Where F_{∥} represents the parallel intensity to the excitation plane, F_{⊥} is the perpendicular intensity to the excitation plane, g is grating factor (G-factor) that represents the intensity ratio of the sensitivity of the detection system for vertically and horizontally polarized light.

In some embodiments, the bioactive peptide amphiphile (e.g. IKVAV peptide amphiphile) comprises a fluorescence anisotropy value of less than 0.3 (e.g. less than 0.3, less than 0.29, less than 0.25, less than 0.24, less than 0.23, less than 0.22, less than 0.21, or less than 0.2).

In some embodiments, the bioactive peptide amphiphile (e.g. IKVAV peptide amphiphile) comprises a relatively low proton relaxation rate (¹H—R₂). Lower proton relaxation rates are indicative of higher motion, thus facilitating the formation of dynamic supramolecular assemblies with high degrees of internal motion. For example, the relaxation rate for the methylene protons attached to the e carbon of the K residue in the IKVAV sequence may be measured by transverse-relaxation nuclear magnetic resonance (T2-NMR) spectroscopy. In some embodiments, the IKVAV peptide amphiphile comprises a proton relaxation rate (¹H—R₂) of less than 4-1. In some embodiments, the IKVAV peptide amphiphile comprises a proton relaxation rate (¹H—R₂) of less than 3-1.

In some embodiments, peptide amphiphiles comprise a non-peptide spacer or linker segment. In some embodiments, the non-peptide spacer or linker segment is located at the opposite terminus of the peptide from the hydrophobic segment. In some embodiments, the spacer or linker segment provides the attachment site for a bioactive group. In some embodiments, the spacer or linker segment provides a reactive group (e.g., alkene, alkyne, azide, thiol, maleimide etc.) for functionalization of the PA. In some embodiments, the spacer or linker is a substantially linear chain of CH₂, O, (CH₂)₂O, O(CH₂)₂, NH, and C═O groups (e.g., CH₂(O(CH₂)₂)₂NH, CH₂(O(CH₂)₂)₂NHCO(CH₂)₂CCH, etc.). In some embodiments, a spacer or linker further comprises additional bioactive groups, substituents, branches, etc. In some embodiments, the linker segment is a single glycine (G) residue.

Suitable peptide amphiphiles for use in the materials herein, as well as methods of preparation of PAs and related materials, amino acid sequences for use in PAs, and materials that find use with PAs, are described in the following patents: U.S. Pat. Nos. 9,044,514; 9,040,626; 9,011,914; 8,772,228; 8,748,569 8,580,923; 8,546,338; 8,512,693; 8,450,271; 8,236,800; 8,138,140; 8,124,583; 8,114,835; 8,114,834; 8,080,262; 8,076,295; 8,063,014; 7,851,445; 7,838,491; 7,745,708; 7,683,025; 7,554,021; 7,544,661; 7,534,761; 7,491,690; 7,452,679; 7,371,719; 7,030,167;
all of which are herein incorporated by reference in their entireties.

The characteristics (e.g., shape, rigidity, hydrophilicity, etc.) of a PA supramolecular structure depend upon the identity of the components of a peptide amphiphile (e.g., lipophilic segment, acidic segment, structural peptide segment, bioactive segment, etc.). For example, nanofibers, nanospheres, intermediate shapes, and other supramolecular structures are achieved by adjusting the identity of the PA component parts. In some embodiments, characteristics of supramolecular nanostructures of PAs are altered by post-assembly manipulation (e.g., heating/cooling, stretching, etc.).

In some embodiments, a peptide amphiphile comprises: (a) a hydrophobic tail comprising an alkyl chain of 8-24 carbons; (b) a structural peptide segment (e.g., comprising A₂G₂(SEQ ID NO: 4) or G₄ (SEQ ID NO: 7)); and (c) a charged segment (e.g., comprising E₂-E₄) In some embodiments, any PAs within the scope described herein, comprising the components described herein, or within the skill of one in the field, may find use herein.

In some embodiments, peptide amphiphiles comprise a bioactive moiety (e.g., IKVAV peptide). In particular embodiments, a bioactive moiety is the most C-terminal or N-terminal segment of the PA. In some embodiments, the bioactive moiety is attached to the end of the charged segment. In some embodiments, the bioactive moiety is exposed on the surface of an assembled PA structure (e.g., nanofiber). A bioactive moiety is typically a peptide, but is not limited thereto.

In some embodiments, the bioactive moiety is a peptide identified in the extracellular matrix (ECM). For example, the bioactive moiety may be a peptide sequence found in collagens, elastins, fibronectins, or laminins. In some embodiments, the bioactive moiety is a peptide sequence found in laminins. For example, the bioactive moiety may be found in laminin-1, laminin-2, laminin-3, laminin-4, laminin-5, laminin-6, laminin-7, laminin-8, laminin-9, laminin-10, laminin-11, laminin-12, laminin-13, laminin-14, or laminin-15. In some embodiments, the bioactive moiety is a peptide sequence found in laminin-1. In particular embodiments, the bioactive moiety is the peptide sequence IKVAV (SEQ ID NO: 1). In some embodiments, the bioactive moiety is a recombinant peptide. In some embodiments, a bioactive moiety is a peptide sequence that binds a peptide or polypeptide of interests, for example, an ECM protein.

In some embodiments, a peptide amphiphile comprises (e.g., from C-terminus to N-terminus or from N-terminus to C-terminus): bioactive peptide (e.g., IKVAV peptide)-charged segment (e.g., comprising E₂₋₄, etc.)-structural peptide segment (e.g., comprising A₂G₂(SEQ ID NO: 4), G₄ (SEQ ID NO: 7))-hydrophobic tail (e.g., comprising an alkyl chain of 8-24 carbons).

In some embodiments, a peptide amphiphile comprises (e.g., from C-terminus to N-terminus or from N-terminus to C-terminus): bioactive peptide (e.g., IKVAV peptide)-flexible linker (e.g. comprising G, etc.)-charged segment (e.g., comprising E₂₋₄, etc.)-structural peptide segment (e.g., comprising A₂G₂(SEQ ID NO: 4), G₄ (SEQ ID NO: 7))-hydrophobic tail (e.g., comprising an alkyl chain of 8-24 carbons).

In some embodiments, provided herein is a bioactive PA comprising IKVAV as the bioactive peptide, also referred to herein as an “IKVAV peptide amphiphile”. In some embodiments the IKVAV peptide amphiphile comprises (e.g., from C-terminus to N-terminus or from N-terminus to C-terminus): IKVAV-charged segment (e.g., comprising E₂₋₄)-structural peptide segment (e.g., comprising A₂G₂, G₄)-hydrophobic tail (e.g., comprising an alkyl chain of 8-24 carbons). In some embodiments, the peptide amphiphile further comprises a linker. For example, in some embodiments, the peptide amphiphile comprises a single glycine residue linking the bioactive peptide (e.g. IKVAV) to the charged peptide segment. In some embodiments, the IKVAV peptide amphiphile comprises C₁₆-A₂G₂E₄GIKVAV (SEQ ID NO: 12). In some embodiments, the IKVAV peptide amphiphile comprises C₁₆G₄E₄GIKVAV (SEQ ID NO: 18).

In some embodiments, the bioactive moiety is a growth factor mimetic peptide. In some embodiments, the growth factor mimetic peptide comprises a growth factor mimetic sequence.

In some embodiments, the growth factor mimetic sequence is a vascular endothelial growth factor (VEGF) mimetic sequence, a fibroblast growth factor 2 (FGF-2) mimetic sequence, a Glial cell-derived neurotrophic factor (GDNF) mimetic sequence, a brain-derived neurotrophic factor (BDNF) mimetic sequence, or a Netrin-1 mimetic sequence. In some embodiments, the growth factor mimetic sequence is an FGF-2 mimetic sequence. In some embodiments, the FGF-2 mimetic sequence comprises YRSRKYSSWYVALKR (SEQ ID NO: 2).

In some embodiments, provided herein is a bioactive PA comprising a growth factor mimetic sequence as the bioactive peptide. Such a peptide amphiphile is referred to herein as a “growth factor mimetic peptide amphiphile”.

In some embodiments, provided herein is a growth factor mimetic peptide amphiphile comprising (e.g., from C-terminus to N-terminus or from N-terminus to C-terminus): growth factor mimetic peptide sequence-charged segment (e.g., comprising E₂₋₄, etc.)-structural peptide segment (e.g., comprising A₂G₂(SEQ ID NO: 4), V₂A₂(SEQ ID NO: 3))-hydrophobic tail (e.g., comprising an alkyl chain of 8-24 carbons). In some embodiments, the peptide amphiphile further comprises In some embodiments, the peptide amphiphile further comprises a linker. For example, in some embodiments, the peptide amphiphile comprises a single glycine residue linking the growth factor mimetic peptide sequence to the charged peptide segment.

In some embodiments, provided herein is a growth factor mimetic peptide amphiphile comprising (e.g., from C-terminus to N-terminus or from N-terminus to C-terminus): a growth factor mimetic peptide sequence-flexible linker (e.g. comprising G, etc.)-charged segment (e.g., comprising E₂₋₄, etc.)-structural peptide segment (e.g., comprising A₂G₂(SEQ ID NO: 4), V₂A₂(SEQ ID NO: 3))-hydrophobic tail (e.g., comprising an alkyl chain of 8-24 carbons). In some embodiments, the growth factor mimetic peptide amphiphile comprises C₁₆-V₂A₂E₄GYRSRKYSSWYVALKR (SEQ ID NO: 13) or C₁₆A₂G₂E₄GYRSRKYSSWYVALKR (SEQ ID NO: 14).

In some embodiments, a PA further comprises an attachment segment or residue (e.g., G) for attachment of the hydrophobic tail to the peptide portion of the PA.

In some embodiments, provided herein are compositions comprising at least two peptide amphiphiles as described herein. In some embodiments, provided herein are compositions comprising at least one IKVAV peptide amphiphile and at least one growth factor mimetic peptide amphiphile. In some embodiments, provided herein is a composition comprising at least one IKVAV peptide amphiphile comprising a hydrophobic segment, a structural peptide segment, a charged peptide segment, and a bioactive peptide comprising the amino acid sequence IKVAV and at least one growth factor mimetic peptide amphiphile. In some embodiments, the IKVAV peptide amphiphile comprises a hydrophobic segment comprising an 8-24 carbon alkyl chain (C₈₋₂₄), a structural peptide segment comprising AAGG (SEQ ID NO: 4), a charged peptide segment comprising E₄(SEQ ID NO: 11), a linker (e.g. G), and the IKVAV (SEQ ID NO: 1) peptide sequence. In some embodiments, the growth factor mimetic peptide amphiphile comprises a hydrophobic segment comprising an 8-24 carbon alkyl chain (C₈₋₂₄), a structural peptide segment comprising V₂A₂(SEQ ID NO: 3) or A₂G₂(SEQ ID NO: 4), a charged peptide segment comprising E₂, E₃, or E₄ (SEQ ID NO: 11), and a growth factor mimetic peptide sequence. In some embodiments, the at least one IKVAV peptide amphiphile and the at least one growth factor mimetic peptide amphiphile interact to form a supramolecular assembly within the composition.

In some embodiments, provided herein are supramolecular assemblies comprising at least two peptide amphiphiles described herein. In some embodiments, a supramolecular assembly is a nanofiber. In some embodiments, provided herein is a supramolecular assembly comprising at least two bioactive peptide amphiphiles as described herein. In some embodiments, provided herein is a supramolecular assembly comprising an IKVAV peptide amphiphile and a growth factor mimetic peptide amphiphile. In some embodiments, provided herein is a supramolecular assembly comprising an IKVAV peptide amphiphile comprising a hydrophobic segment, a structural peptide segment, a charged peptide segment, and a bioactive peptide comprising the amino acid sequence IKVAV and a growth factor mimetic peptide amphiphile. In some embodiments, the IKVAV peptide amphiphile comprises a hydrophobic segment comprising an 8-24 carbon alkyl chain (C₈₋₂₄), a structural peptide segment comprising AAGG (SEQ ID NO: 4), a charged peptide segment comprising E₄ (SEQ ID NO: 11), a linker (e.g. G), and the IKVAV peptide sequence (SEQ ID NO 1). In some embodiments, the growth factor mimetic peptide amphiphile comprises a hydrophobic segment comprising an 8-24 carbon alkyl chain (C₈₋₂₄), a structural peptide segment comprising V₂A₂(SEQ ID NO: 3) or A₂G₂(SEQ ID NO: 4), a charged peptide segment comprising E₂, E₃, or E₄ (SEQ ID NO: 11), and a growth factor mimetic peptide sequence.

In some embodiments, supramolecular assemblies (e.g. nanostructures, such as nanofibers) are assembled from a first type of PA bearing a bioactive moiety (e.g., IKVAV peptide amphiphiles) and a growth factor mimetic PA. In some embodiments, the compositions or supramolecular assemblies (e.g. nanostructures, such as nanofibers) described herein comprise a molar ratio of IKVAV PAs to growth factor mimetic PAs of about 90:10. In some embodiments, the molar ratio of IKVAV PA:growth factor mimetic PA is about 99:1, about 98:2, about 97:3, about 96:4, about 95:5, about 94:6, about 93:7, about 92:8, about 91:9: about 90:10, about 89:11, about 88:12, abut 87:13, about 86:14, or about 85:15. In some embodiments, the ratio of IKVAV PA to growth factor mimetic determines the mechanical characteristics (e.g., liquid or gel) of the nanofiber material and under what conditions the material will adopt various characteristics (e.g., gelling upon exposure to physiologic conditions, liquifying upon exposure to physiologic conditions, etc.).

In some embodiments, the compositions and supramolecular assemblies described herein additionally describe one or more filler PAs. The term “filler PA” or “diluent PA” are used interchangeably herein to refer to a PA comprising a hydrophobic segment, a structural peptide segment, and a charged peptide segment as described herein, but lacking a bioactive moiety (e.g. lacking the IKVAV peptide sequence, lacking the growth factor mimetic peptide sequence, etc.). In some embodiments, a filler PA is a non-bioactive PA molecule having highly charged glutamic acid residues on the terminal end of the molecule (e.g., surface-displayed end). These negatively charged PAs allow for the gelation to take place between nanofibers via ionic crosslinks. In some embodiments, a filler PA is a non-bioactive PA molecule having highly charged lysine residues on the terminal end of the molecule (e.g., surface-displayed end). These positively charged PAs allow for the gelation to take place under basic conditions. The filler PAs provide the ability to incorporate other bio-active PAs molecules into the nanofiber matrix while still ensuring the ability of the nanofibers solution to gel. In some embodiments, the solutions are annealed for increased viscosity and stronger gel mechanics. These filler PAs have sequences are described in, for example, U.S. Pat. No. 8,772,228 (e.g., C₁₆-VVVAAAEEE (SEQ ID NO: 19)), which is herein incorporated by reference in its entirety.

In some embodiments, the PA nanofiber described herein exhibit a small cross-sectional diameter (e.g., <25 nm, <20 nm, <15 nm, about 10 nm, etc.). In some embodiments, the small cross-section of the nanofibers (˜10 nm diameter) allows the fibers to permeate the brain parenchyma.

In some embodiments, the PAs, compositions, and supramolecular assemblies described herein find use in treating or preventing a nervous system injury in a subject. In some embodiments, the PAs, compositions, and supramolecular assemblies (e.g. nanofibers) described herein may be used for methods of treatment of nervous system injury in a subject. For example, the PAs, compositions, and supramolecular assemblies described herein may be used in methods for treatment of prevention of injury to the central nervous system (CNS), including the brain and the spinal cord, or the peripheral nervous system (PNS), including the nerves and ganglia outside of the brain and spinal cord. In some embodiments, the PAs, compositions, and supramolecular assemblies described herein may be used for treatment or prevention of injury to the CNS or PNS in a subject. In some embodiments, the injury is a spinal cord injury. The spinal cord injury may be cervical, lumbar, thoracic, sacral, or any combination thereof.

The injury may be a traumatic injury. A traumatic injury refers to an injury caused by trauma, for example trauma such as that caused by an automobile accident, a fall, violence, sports injury, surgical injury, and the like.) For example, the PAs, compositions, and supramolecular assemblies described herein may be used for the treatment of traumatic spinal cord injury. As another example, the PAs, compositions, and supramolecular assemblies described herein may be used for the treatment of traumatic brain injury (TBI). Alternatively, the injury may be a non-traumatic injury. For example, the injury may be a non-traumatic injury to the CNS (e.g., the brain and/or the spinal cord) or the PNS caused by, for example, cancer, multiple sclerosis, inflammation, arthritis, spinal stenosis, tumors, blood loss, and the like.

In some embodiments, the composition comprising PAs and/or supramolecular assemblies (e.g. nanofibers) as described herein is provided to a subject suspected of having a traumatic spinal cord injury. For example, the composition may be provided to the subject exhibiting one or more symptoms including loss of sensation and/or loss of motor control in one or more areas of the body (e.g. hands, arms, legs, feet, etc.), low blood pressure, inability to regulate blood pressure, inability to regulate body temperature, inability to sweat below the area of injury, chronic pain, and/or swelling of the spinal cord. The composition may be provided to the subject to treat the injury. In some embodiments, treating the injury may prevent worsening of one or more symptoms associated with the injury. In some embodiments, treating the injury may reduce the severity of and/or eliminate one or more symptoms associated with the injury. In some embodiments, the composition is used to promote vascularization, nerve regeneration, functional recovery, and/or to limit the damage after spinal cord injury.

The composition may be provided to a subject at any suitable point following injury (e.g. traumatic spinal cord injury) to treat the injury. For example, the composition may be provided to the subject within 24 hours of the injury (e.g. within 24 hours, within 12 hours, within 10 hours, within 9 hours, within 8 hours, within 7 hours, within 6 hours, within 5 hours, within 4 hours, within 3 hours, within 2 hours, or within 1 hour from injury. In some embodiments, the composition may be provided to the subject after a duration longer than 24 hours has passed following injury or diagnosis of injury.

The composition may be administered in any suitable amount, depending on factors including the age of the subject, weight of the subject, severity of the injury, and the like. The composition may be administered in combination with other suitable treatments for injury or preventative measures to prevent the severity of the injury from worsening.

In some embodiments, compositions described herein are formulated for delivery to a subject. Suitable routes of administrating the composition described herein include, without limitation: topical, subcutaneous, transdermal, intradermal, intralesional, intraarticular, intraperitoneal, intravesical, transmucosal, gingival, intradental, intracochlear, transtympanic, intraorgan, epidural, intrathecal, intramuscular, intravenous, intravascular, intraosseus, periocular, intratumoral, intracerebral, and intracerebroventricular administration. In some embodiments, the PA compositions are administered parenterally. In some embodiments, parenteral administration is by intrathecal administration, intracerebroventricular administration, or intraparenchymal administration. The PA compositions herein can be administered as the sole active agent or in combination with other pharmaceutical agents such as other agents used in the treatment of nervous system injury in a subject.

### Example 1

### Supramolecular Motion in Bioactive Scaffolds Promotes Recovery from Spinal Cord Injury

Overview: Described herein are peptide amphiphile supramolecular polymers containing two distinct signals. These supramolecular polymers were tested in a mouse model of severe spinal cord injury. One signal activates the transmembrane receptor b1-integrin and a second one activates the basic fibroblast growth factor 2 receptor. By mutating the peptide sequence of the amphiphilic monomers in nonbioactive domains, the motions of molecules within scaffold fibrils were intensified, which resulted in notable differences in vascular growth, axonal regeneration, myelination, survival of motor neurons, reduced gliosis, and functional recovery. Accordingly, the signaling of cells by ensembles of molecules can be optimized by tuning their internal motions.

### Introduction

Pharmacological signaling of cells usually proceeds through strong binding of small organic molecules to proteins that activate or inhibit particular responses. An emerging signaling strategy is to use nanostructures that target specific cells to deliver a therapeutic cargo, or materials functioning as bioactive scaffolds in the extracellular space. Less developed aspects of this field is the molecular design of materials bearing signals for receptors and the connections between such signals and the motions of molecules within artificial scaffolds. Described herein is a supramolecular scaffold of nanoscale fibrils that integrates two different orthogonal biological signals, the laminin signal IKVAV which promotes differentiation of neural stem cells into neurons and to extend axons, and the fibroblast growth factor-2 (FGF-2) mimetic peptide YRSRKYSSWYVALKR (SEQ ID NO: 2), which activates the receptor FGFR1 to promote cell proliferation and survival. The two signals were placed at the termini of two different peptides with alkyl tails, known as peptide amphiphiles (PAs), that copolymerize noncovalently in aqueous media to form supramolecular fibrils. Different domains that alter the physical properties of a potential scaffold therapy to restore functional recovery in vivo after hind limb paralysis in a murine model of severe spinal cord injury (SCI) were investigated herein. The development of SCI therapies that avoid permanent paralysis in humans after traumatic injuries remains a major challenge given the inability of damaged axons to regenerate in the adult central nervous system (CNS). Herein, it was discovered that keeping both biological signals at the same density, but slightly mutating the tetrapeptide sequence of these domains, could dramatically change the biological responses of cells in vitro as well as the functional recovery from SCI in mice in vivo.

### Results

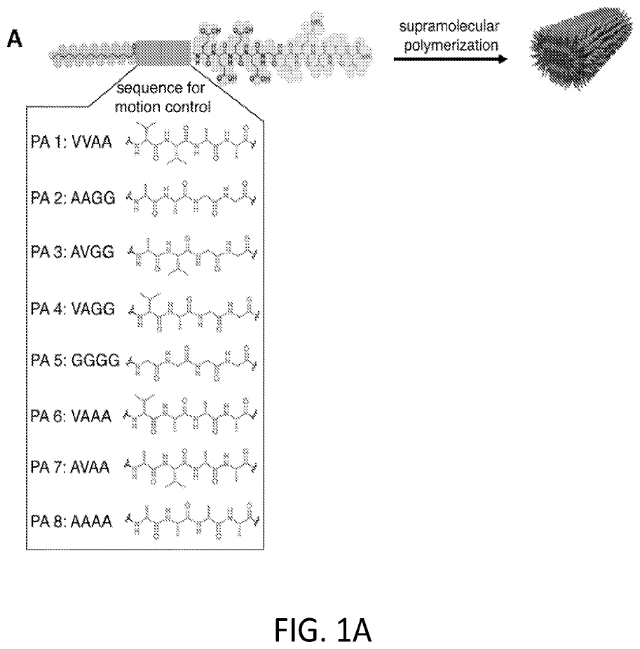

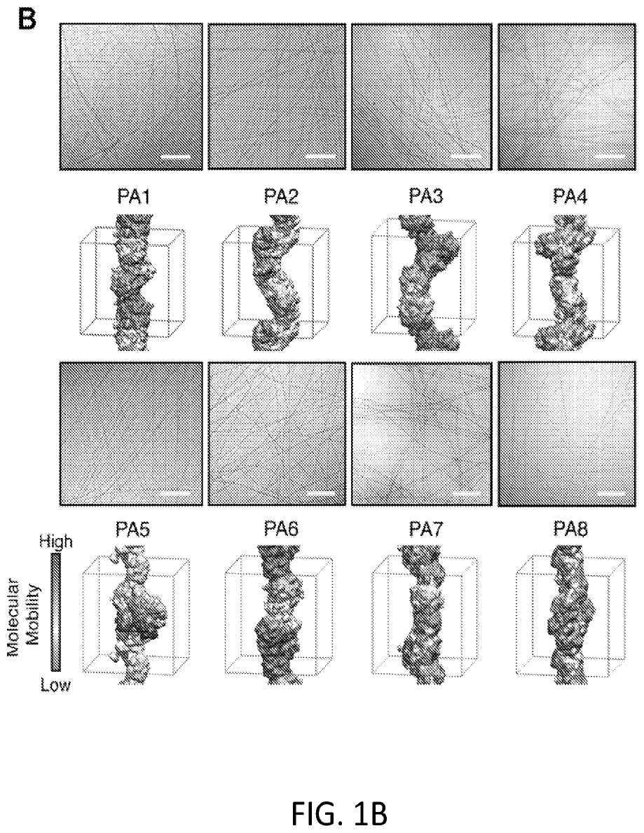

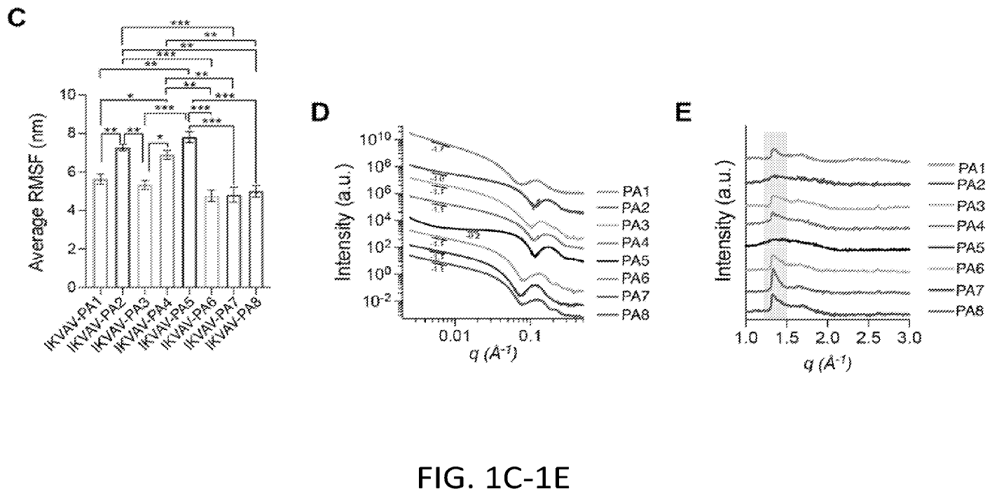

In order to investigate nanofiber-shaped supramolecular polymers with different physical properties that display the same two signals, a library of different IKVAV PAs was synthesized in which the tetrapeptide domain controlling physical behavior has different sequences of the amino acids V, A and G (IKVAV PA1-PA8) (see FIG. 1A, FIG. 7, and Table 5 for the list of PAs used and their peptide sequences). These amino acids were selected because they affect the propensity of molecules within the fibrils to form β-sheets, which have high intermolecular cohesion as a result of their hydrogen-bond density. These interactions in turn results in suppressed mobility of PA molecules within the fibril. For example, V₂A₂(PA1) has a high propensity to form β-sheet structure because of its valine content whereas A₂G₂(PA2) is potentially a less ordered segment without secondary structure (see FIG. 1A). The rest of the sequences were selected as potential candidates for an intermediate level of motion. All IKVAV PAs utilized the sequence E₄G, which spaces this segment from the bioactive signal and provides high solubility in water.

TABLE 5
List of PAs used and Peptide Sequences

| Sample Name | Peptide Sequence |
|---|---|
| IKVAV PA1 | C₁₆-V₂A₂E₄GIKVAV (SEQ ID NO: 20) |
| IKVAV PA2 | C₁₆-A₂G₂E₄GIKVAV (SEQ ID NO: 12) |
| IKVAV PA3 | C₁₆-AVG₂E₄GIKVAV (SEQ ID NO: 21) |
| IKVAV PA4 | C₁₆-VAG₂E₄GIKVAV (SEQ ID NO: 22) |
| IKVAV PA5 | C₁₆-G₄E₄GIKVAV (SEQ ID NO: 18) |
| IKVAV PA6 | C₁₆-VA₃E₄GIKVAV (SEQ ID NO: 23) |
| IKVAV PA7 | C₁₆-AVA₂E₄GIKVAV (SEQ ID NO: 24) |
| IKVAV PA8 | C₁₆-A₄E₄GIKVAV (SEQ ID NO: 25) |
| Backbone PA | C₁₆-V₂A₂E₂ (SEQ ID NO: 26) |
| FGF2 PA1 | C₁₆-V₂A₂E₄GYRSRKYSSWYVALKR (SEQ ID |
| | NO: 13) |
| FGF2 PA2 | C₁₆-A₂G₂E₄GYRSRKYSSWYVALKR (SEQ ID |
| | NO: 14) |
| VVIAK PA1 | C₁₆-V₂A₂E₄GVVIAK (SEQ ID NO: 27) |
| VVIAK PA2 | C₁₆-A₂G₂E₄GVVIAK (SEQ ID NO: 28) |
| scrFGF2 PA1 | C₁₆-V₂A₂E₄GWRSKKYSLYYVASRR (SEQ ID |
| | NO: 29) |
| scrFGF2 PA2 | C₁₆-A₂G₂E₄GWRSKKYSLYYVASRR (SEQ ID |
| | NO: 30) |
| Alexa647 IKVAV PA2 | C₁₆-A₂G₂E₄GIKVAV (SEQ ID NO: 12)-Alexa647 |
| FGF2 PA1Cy3 | C₁₆-V₂A₂E₄GYRSRKYSSWYVALKR (SEQ ID |
| | NO: 13)-Cy3 |
| FGF2 PA2Cy3 | C₁₆-A₂G₂E₄GYRSRKYSSWYVALKR( SEQ ID |
| | NO: 14)-Cy3 |

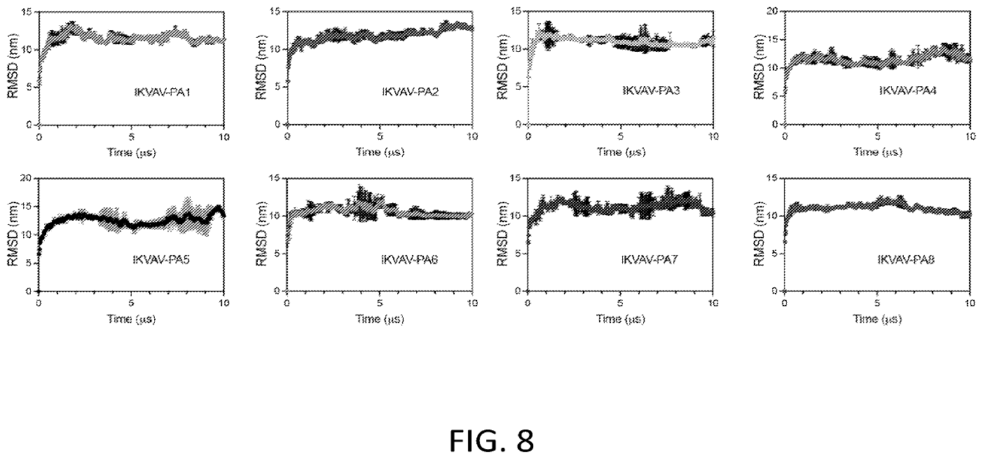

Cryogenic transmission electron microscopy (Cryo-TEM) revealed that all IKVAV PAs formed nanofibers after supramolecular polymerization in water (FIG. 1B). Furthermore, synchrotron solution small-angle x-ray scattering (SAXS) confirmed the formation of filaments revealing a slope in the range −1 to −1.7 in the Guinier region except for PA5, which suggests a mixture of filaments and spherical micelles (slope=−0.2) (FIG. 1D). The physical behavior of the various assemblies in the library was compared using coarse-grained molecular dynamic (CG-MD) simulations using the MARTINI force field (13) (FIG. 8). These simulations predicted that molecules within the various IKVAV PA fibers had different degrees of internal dynamics (FIG. 1B). Differences in the ability of the molecules to change positions internally over appreciable distances (on the order of nanometers) were suggested by the simulations, which yielded values of the parameter defined as the root-meansquare fluctuation (RMSF), which is a measure of the average displacement of a PA molecule during the last 5 ms of the simulation (FIG. 1C). These simulations indicate that molecules in PA2 fibers indeed have a high degree of internal motion, as well as PA5 which only contains G residues. Wide-angle X-ray analysis (WAXS) also revealed the presence of internal order (β-sheet Bragg peak with a d-spacing of 4.65 Å) in all the IKVAV PAs except for those with low RMSF values (PA2 and PA5) (FIG. 1E).

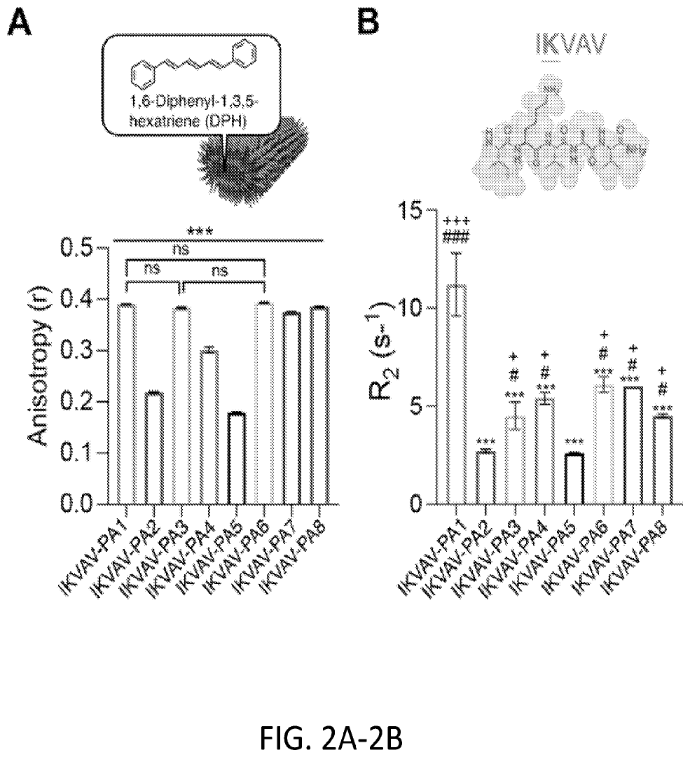

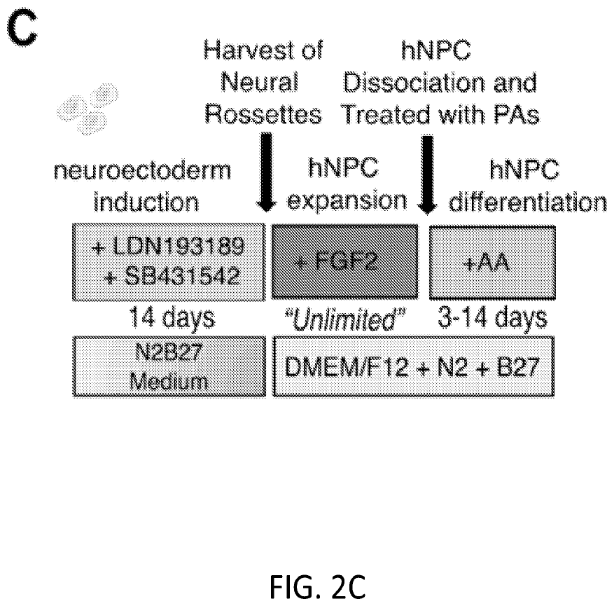

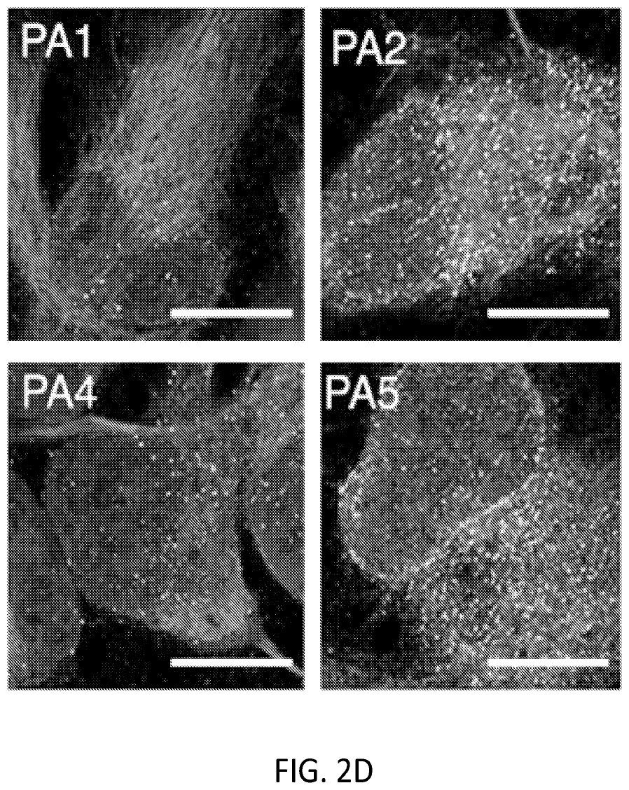

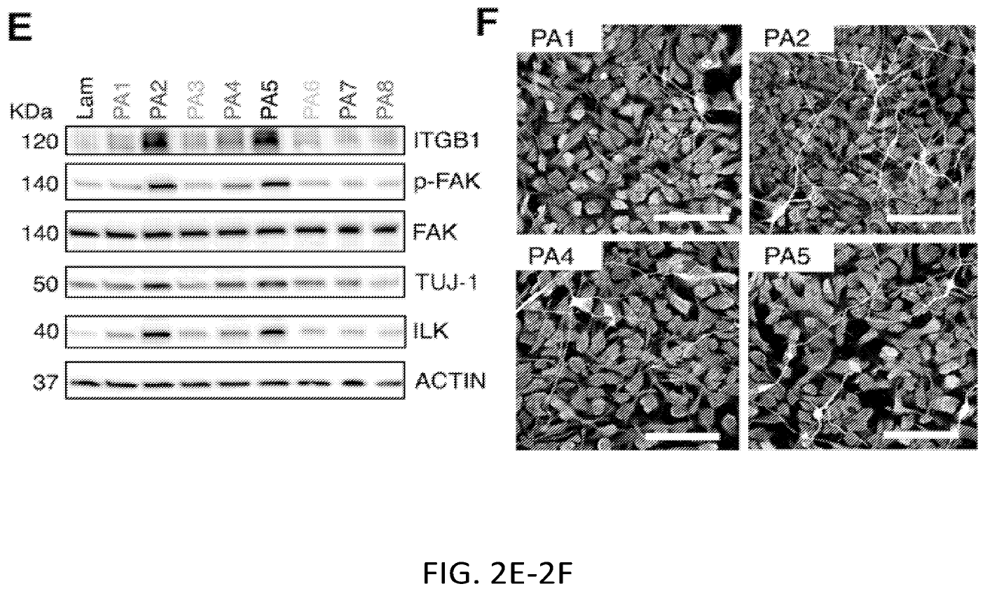

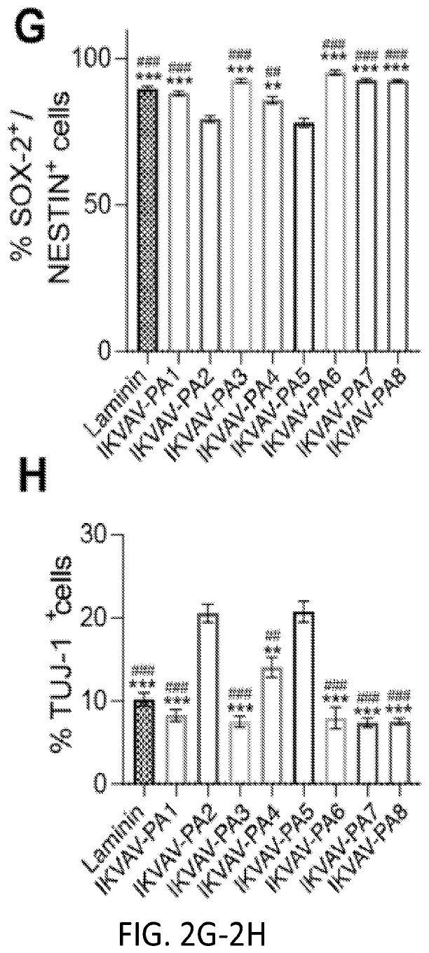

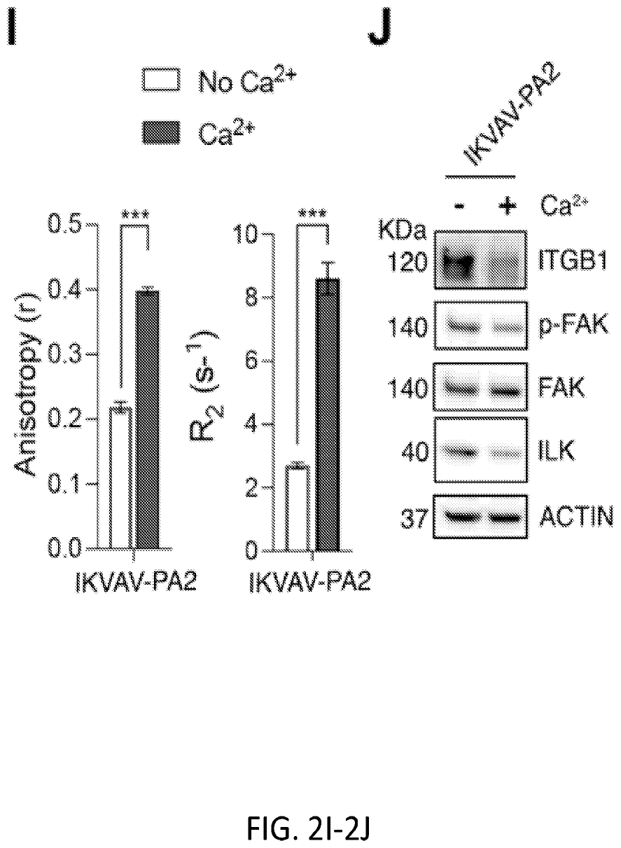

In order to probe differences in dynamics among the IKVAV PAs, fluorescence depolarization (FD) measurements were taken by encapsulating 1,6-diphenyl-1,3,5-hexatriene (DPH) within PA nanofibers to measure the microviscosity of the inner hydrophobic core. PA2 and PA5 had the lowest anisotropy values (0.21 and 0.18, respectively) indicating they formed the most dynamic supramolecular assemblies, PA4 had intermediate dynamics (0.30), and the remaining PAs had less intense supramolecular motion (0.40 to 0.37) (FIG. 2A). Molecular dynamics in the IKVAV epitope were also measured using transverse-relaxation nuclear magnetic resonance (T2-NMR) spectroscopy. These experiments obtained the relaxation rate for the methylene protons attached to the e carbon (He) of the K residue in the IKVAV sequence (observed at 2.69 to −2.99 parts per million). IKVAV PA1 showed the highest relaxation rate (a low degree of motion), whereas IKVAV PA2 and PA5 had the lowest relaxation rates in the IKVAV PA library (¹H—R₂=2.7±0.1 and 2.6±0.003 s⁻¹, respectively, consistent with greater motion (FIG. 2B and Table 2). Consistent with FD results, IKVAV PA4 reveals an intermediate level of supramolecular motion between PA1 and PA2 (or PA5). Collectively, the simulations as well as FD, WAXS, and T2-NMR measurements are consistent with three levels of supramolecular motion in the library of molecules investigated.

TABLE 2
Library of IKVAV PAs and their T2 Relaxation Times.

| Sample Name | Tetrapeptide Sequence | □_{H} (ppm) | R2 (/s) |
|---|---|---|---|
| IKVAV PA1 | VVAA (SEQ ID NO: 3) | 2.72 | 11.2 ± 2.9 |
| IKVAV PA2 | AAGG (SEQ ID NO: 4) | 2.87 | 2.7 ± 0.1 |
| IKVAV PA3 | AVGG (SEQ ID NO: 5) | 2.69 | 4.5 ± 0.7 |
| IKVAV PA4 | VAGG (SEQ ID NO: 6) | 2.77 | 5.6 ± 0.4 |
| IKVAV PA5 | GGGG (SEQ ID NO: 7) | 2.79 | 2.6 ± 0.03 |
| IKVAV PA6 | VAAA (SEQ ID NO: 8) | 2.78 | 6.1 ± 0.8 |
| IKVAV PA7 | AVAA (SEQ ID NO: 9) | 2.81 | 6.0 ± 0.03 |
| IKVAV PA8 | AAAA (SEQ ID NO: 10) | 2.99 | 4.5 ± 0.2 |
| IKVAV PA2(+Ca²⁺) | AAGG (SEQ ID NO: 4) | 2.87 | 8.6 ± 0.5 |
| | (+Ca²⁺) | | |

### Supramolecular Motion and In Vitro Bioactivity

In vitro experiments were performed to determine if the IKVAV signal was equally bioactive in the library of IKVAV PAs. To establish the bioactivity of IKVAV PAs, neural progenitor cells derived from human embryonic stem cells (hNPCs) were treated either with the different IKVAV PA fibers in solution or the recombinant protein laminin (FIG. 2C). PA filaments associate closely with cells and can activate receptors when their surfaces display signals.

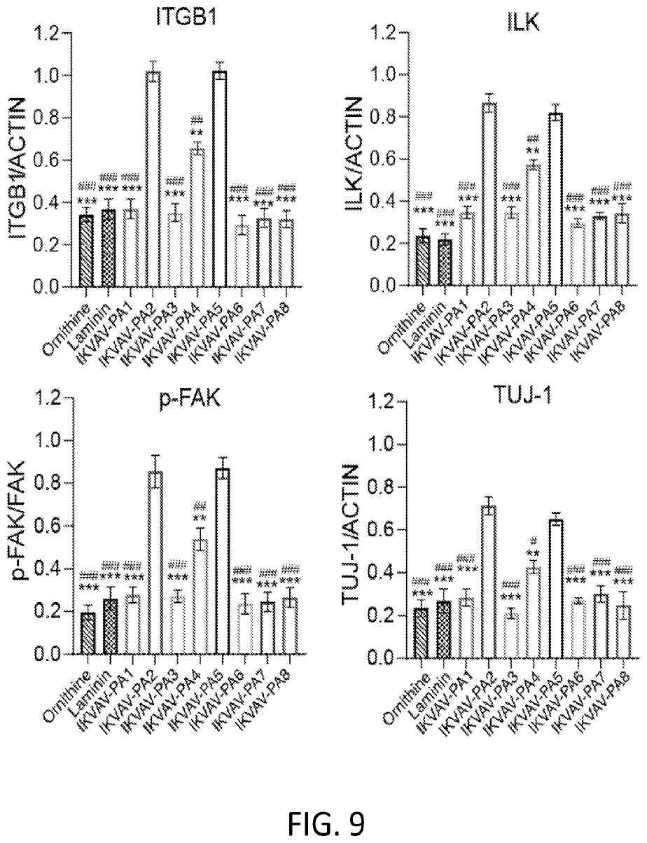

The activation of the transmembrane receptor β1-INTEGRIN (ITGB1) expressed in the presence of IKVAV PAs and laminin was evaluated using the active form-specific antibody HUTS4. The activation of the receptor's intracellular signaling pathway was also verified. Fluorescence confocal microscopy and western blot (WB) analysis showed that IKVAV PA2 and PA5 induced substantially higher concentrations of active ITGB1 and the downstream effectors integrin-linked kinase (ILK) and phospho-focal adhesion kinase (p-FAK) relative to the rest of the IKVAV PAs, the IKVAV peptide, and laminin or ornithine coatings as controls (FIGS. 2, D and E, and FIG. 9). An intermediate level of activation with PA4 was observed that correlated with its intermediate supramolecular motion relative to the rest of the PAs in the library. PAs displaying the VVIAK scrambled sequence resulted in minimal cellular activation of ITGB1. Furthermore, pre-treatment with an ITGB1 antibody blocked the attachment of hNPCs on all IKVAV PAs, suggesting that an IKVAV-ITGB1 interaction mediated this process.

Although hNPCs upregulated the neuronal form of β-TUBULIN (TUJ-1⁺) when treated with IKVAV PAs, this induction (which reflects neuronal differentiation commitment) was higher for IKVAV PA2 and PA5 (20.5±1% and 20.7±1.2%, respectively), the two most dynamic supramolecular fibrils (FIG. 2, F to H). The other IKVAV PAs, with the exception of IKVAV PA4 which showed an intermediate neuronal differentiation commitment (PA4: 14±1.2%), had a lower percentage of induction of TUJ-1⁺ neuronal cells (PA1: 8.2±0.7%, PA3: 7.5±0.6%, PA6: 7.9±1.3%, PA7: 7.4±0.6%, and PA8: 7.5±0.5%). By using puromycin-based protein synthesis analysis (SUnSET technique), it was verified that all of the conditions showed similar protein translation levels, so the observed differences were not linked to a metabolic effect.

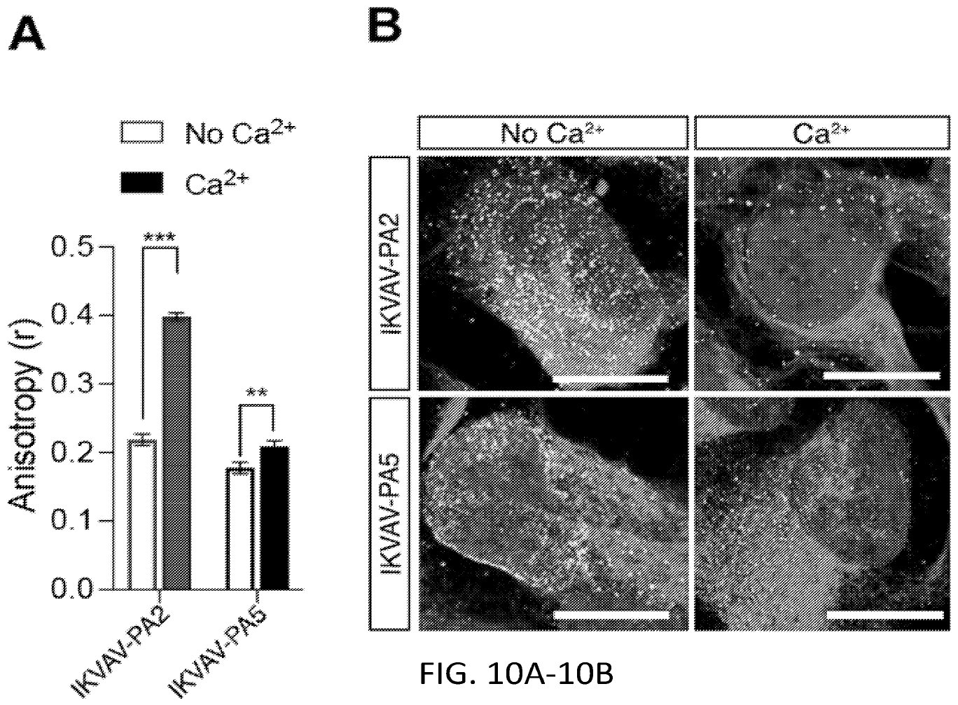

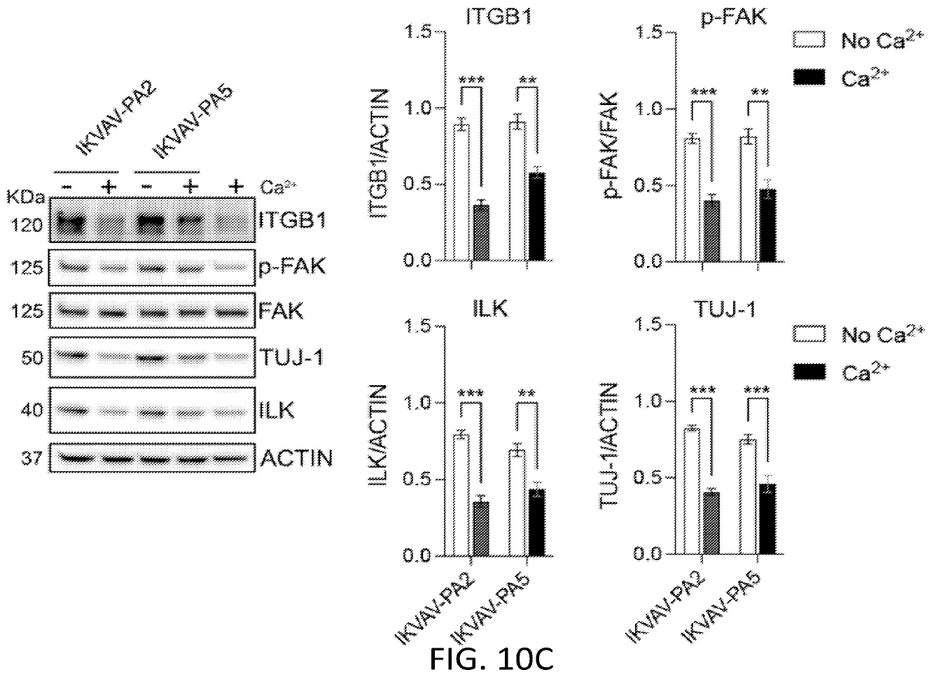

In vitro experiments were performed in which hNPCs were treated with the most bioactive IKVAV PAs (PA2 and PA5) mixed with 5 mM CaCl₂) which electrostatically cross-links with negatively charged PA fibers. The addition of Ca²⁺ suppressed supramolecular motion, which was confirmed by FD and T2-NMR experiments (FIG. 2I). When supramolecular motion was decreased by adding Ca²⁺ ions to the media, the activation of ITGB1 and its downstream intracellular pathway (ILK, p-FAK/FAK) also decreased (FIG. 2J and FIG. 10a-10c). These results showed a strong positive correlation between dynamics and in vitro bioactivity as mutations were introduced in the tetrapeptide amino acid sequence in the non-bioactive domain of IKVAV PAs.

### SCI Model: Axon Regrowth and Formation of Glial Scar

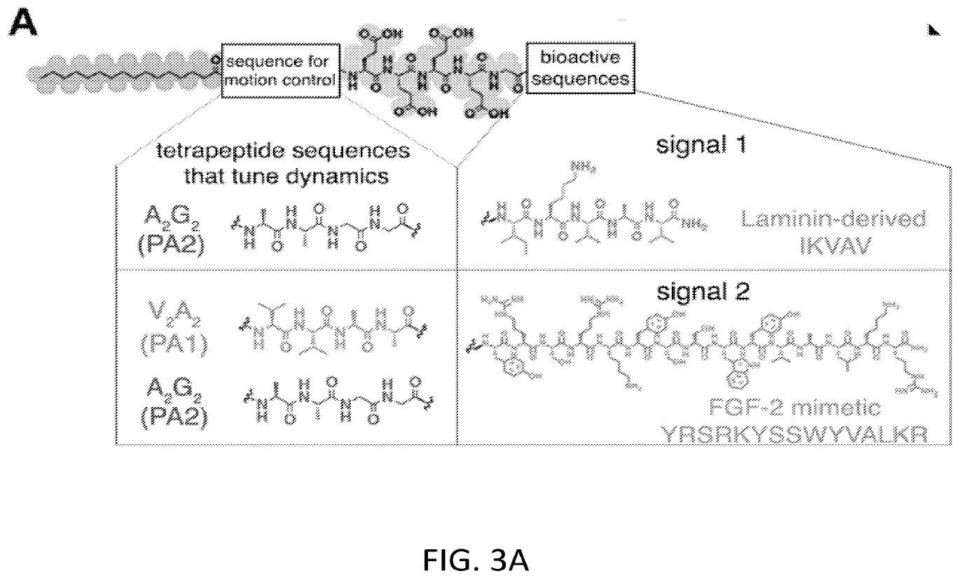

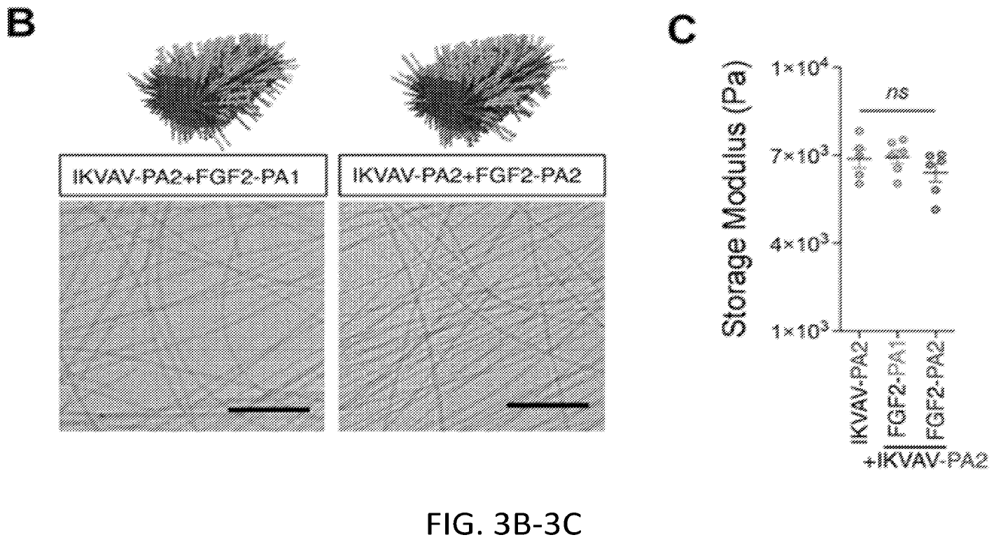

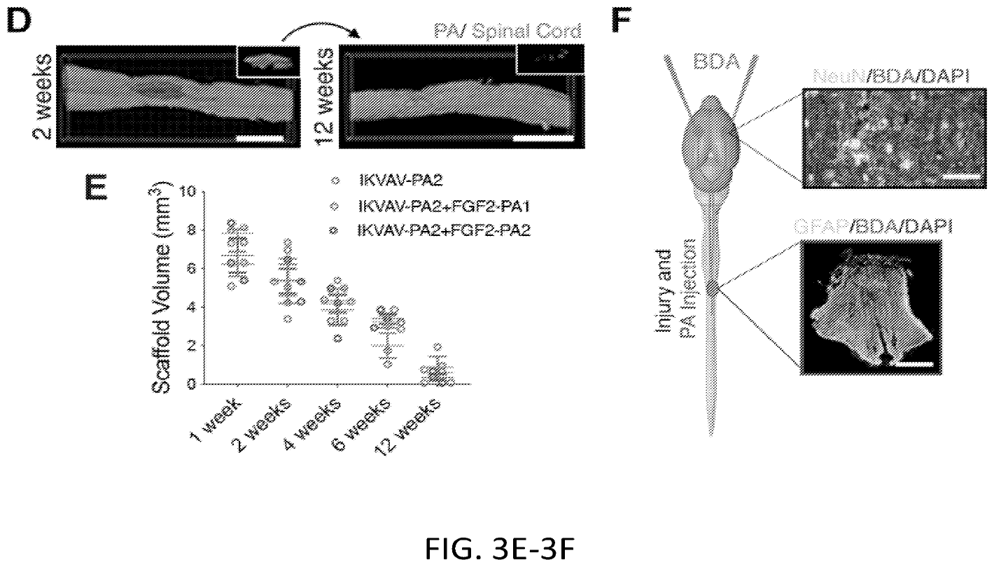

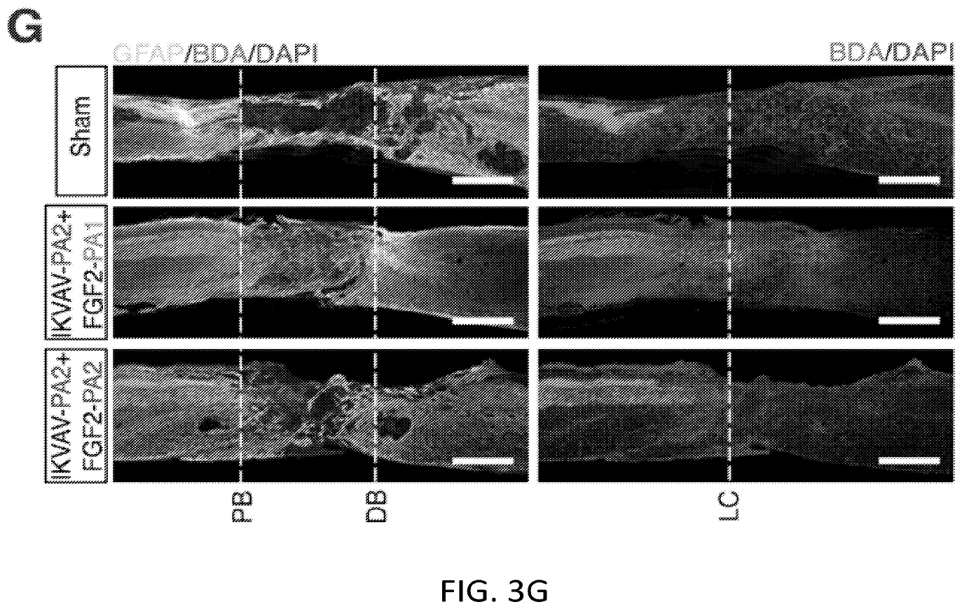

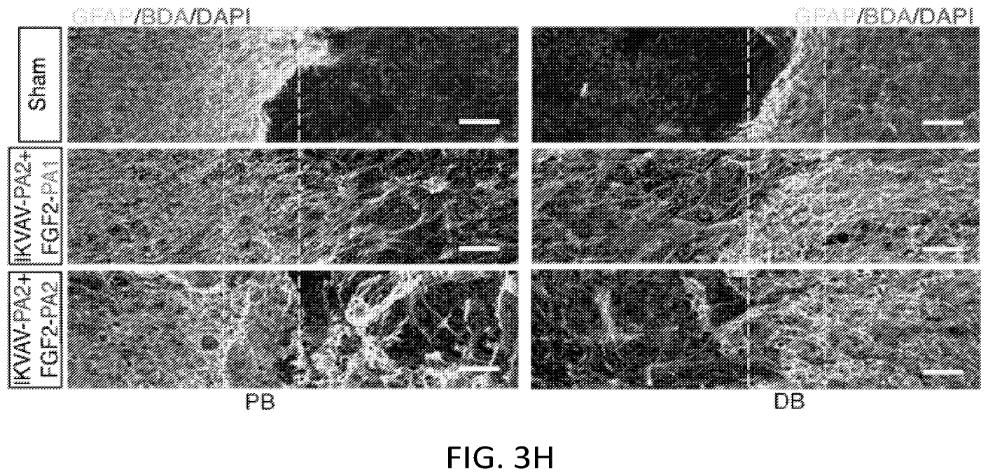

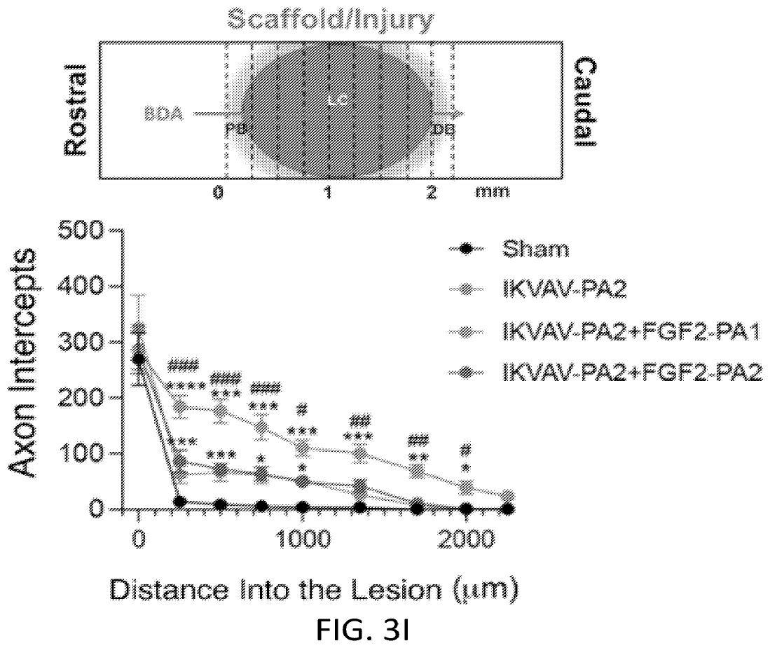

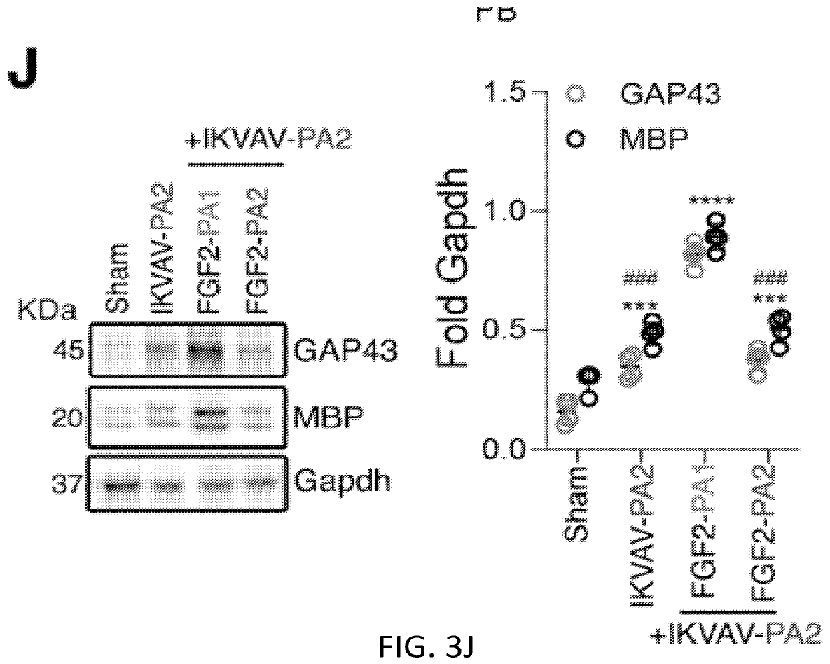

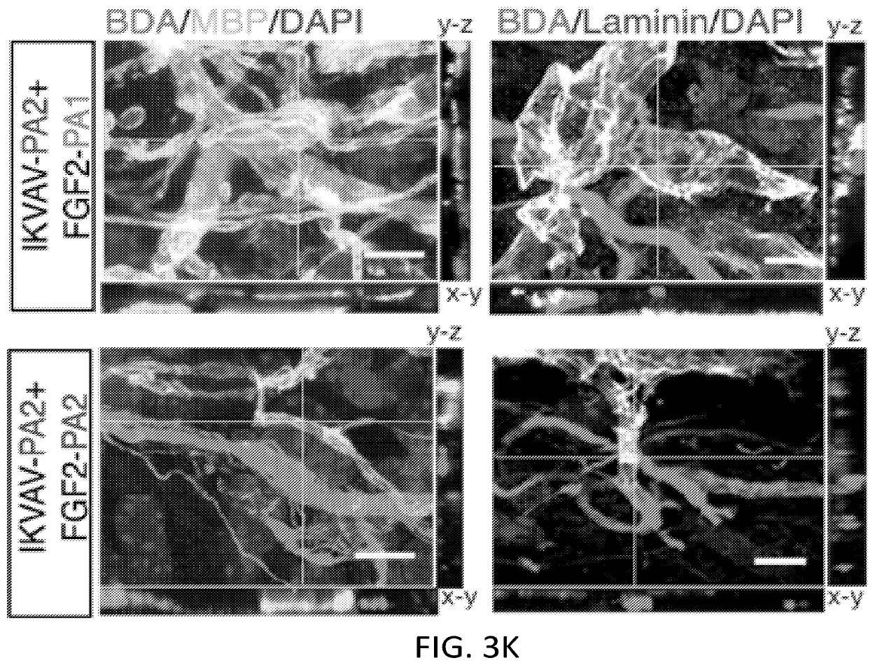

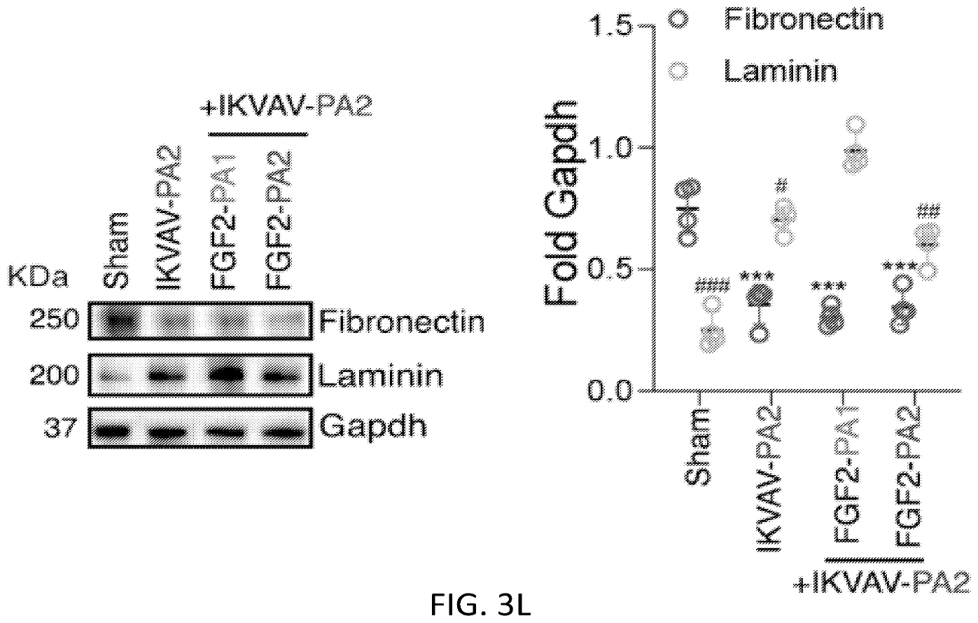

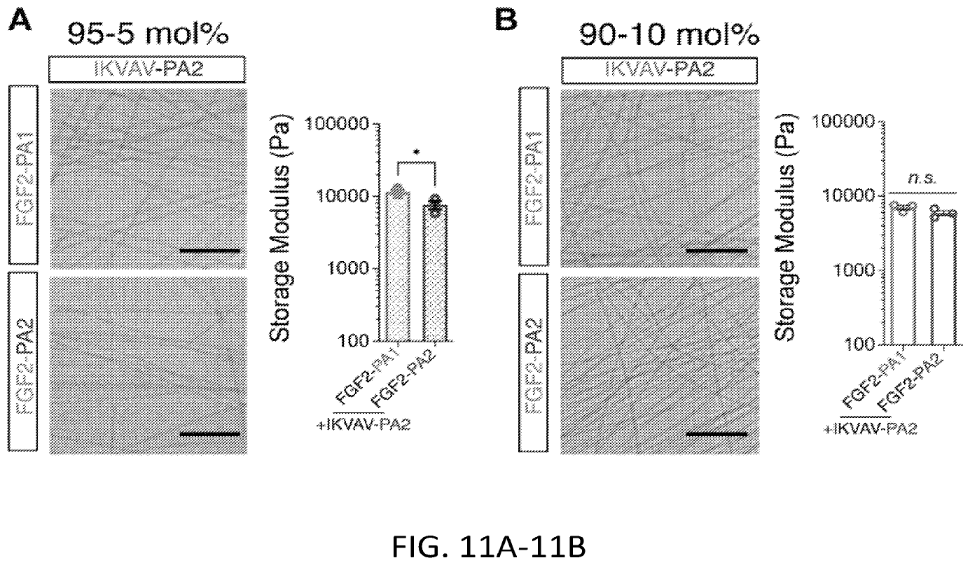

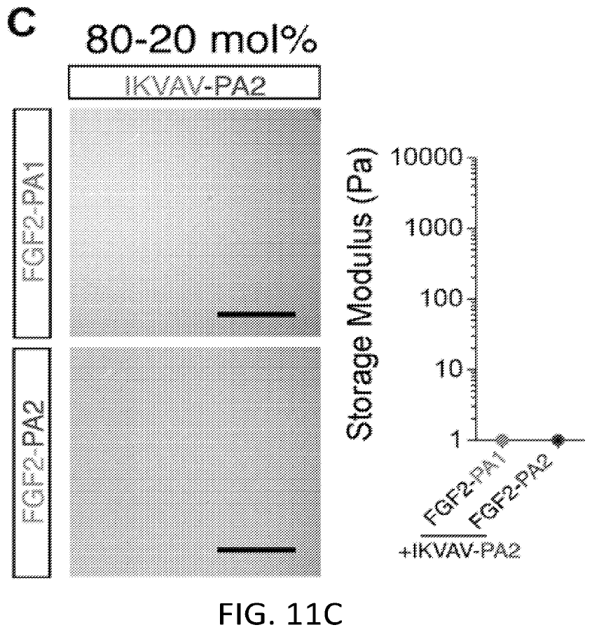

Next, the ability of dual signal fibrils to enhance functional recovery after SCI in vivo was tested. Given the low level of in vitro bioactivity observed for IKVAV PA1, PA3, PA4, PA6, PA7 and PA8, these PAs were not used in combination with the FGF2 PAs. Nanofibers that display both signals simultaneously were also tested, so the binary systems had to be miscible and form hydrogels with similar mechanical properties upon contact with physiological fluids once injected at the site of the injury. Only IKVAV PA2 was both miscible and could form hydrogels with similar mechanical properties when mixed with either FGF2 PA1 or FGF2 PA2, particularly at a molar ratio of 90:10 (FIG. 3, A to C, FIG. 11A-11C, Table 3). Furthermore, both FGF2 PAs alone formed highly aggregated short fibers that further contributed to immiscibility with other IKVAV PAs such as PA1, PA4 or PA5.

TABLE 3
List of PA co-assemblies used.

| | Co-Assembly Ratios Used (Mol %) | | | |
|---|---|---|---|---|
| Sample Name (A + B + . . . ) | A | B | C | D |
| IKVAV PA1 + FGF2 PA1 | 90% or 95% | 10% or 5% | — | — |
| IKVAV PA1 + FGF2 PA2 | 90% or 95% | 10% or 5% | — | — |
| IKVAV PA2 + FGF2 PA1 | 90% or 95% | 10% or 5% | — | — |
| IKVAV PA2 + FGF2 PA2 | 90% or 95% | 10% or 5% | — | — |
| IKVAV PA4 + FGF2 PA1 | 90% | 10% | — | — |
| IKVAV PA4 + FGF2 PA2 | 90% | 10% | — | — |
| IKVAV PA5 + FGF2 PA1 | 90% | 10% | — | — |
| IKVAV PA5 + FGF2 PA2 | 90% | 10% | — | — |
| VVIAK PA2 + scrFGF2 PA1 | 90% | 10% | — | — |
| VVIAK PA2 + scrFGF2 PA2 | 90% | 10% | — | — |
| Alexa-647 IKVAV PA2 + IKVAV | 1% | 89% | 1% | 9% |
| PA2 + Cy3 FGF2 PA1 + FGF2 | | | | |
| PA1 | | | | |
| Alexa-647 IKVAV PA2 + IKVAV | 1% | 89% | 1% | 9% |
| PA2 + Cy3 FGF2 PA2 + FGF2 | | | | |
| PA2 | | | | |

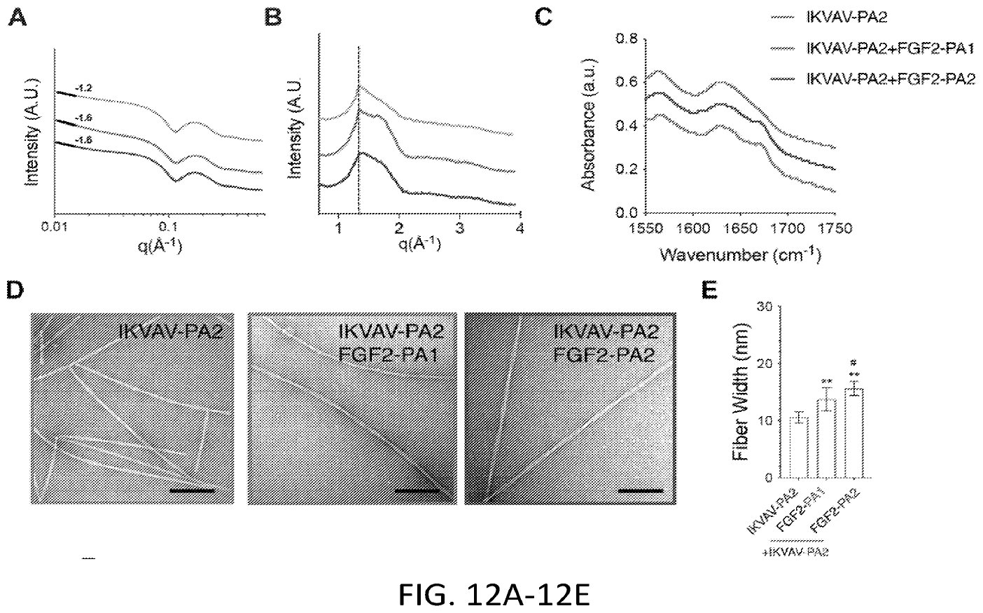

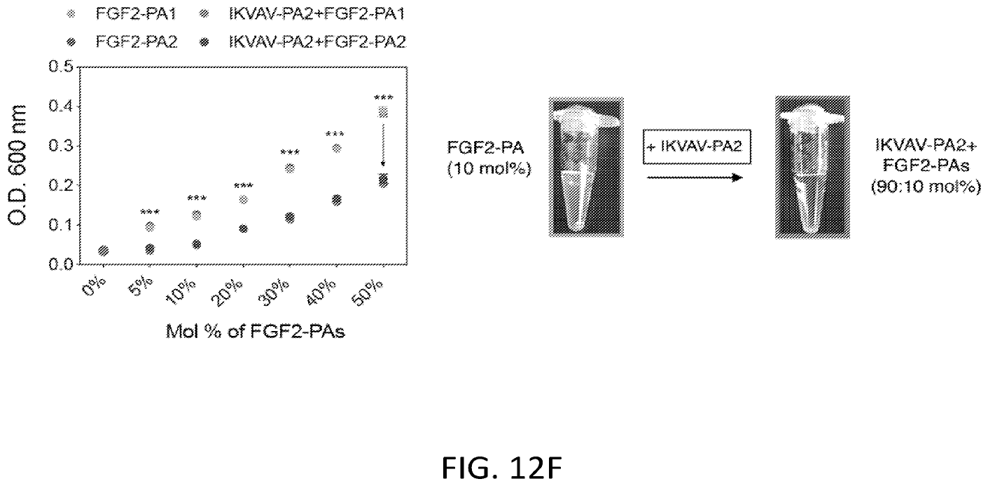

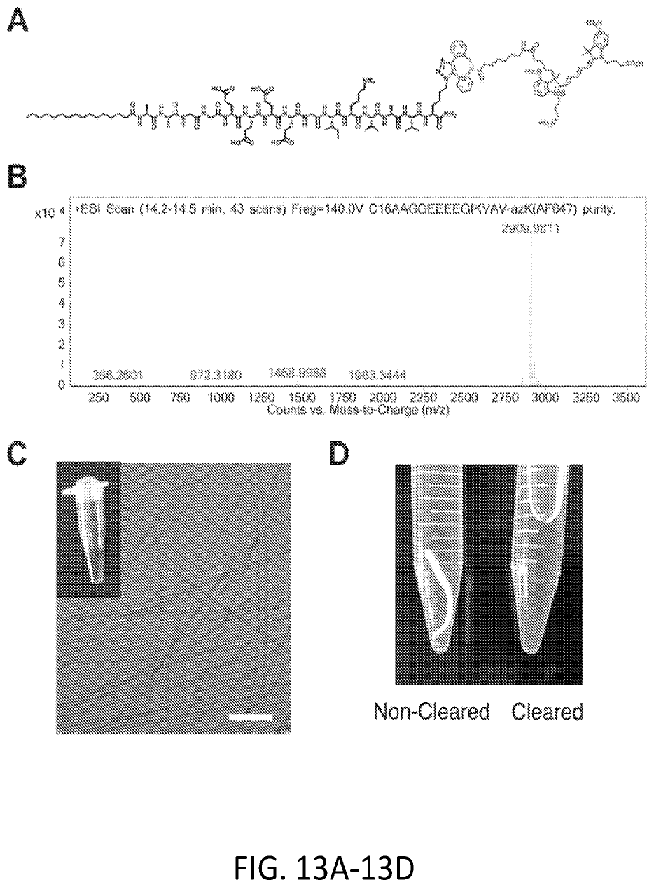

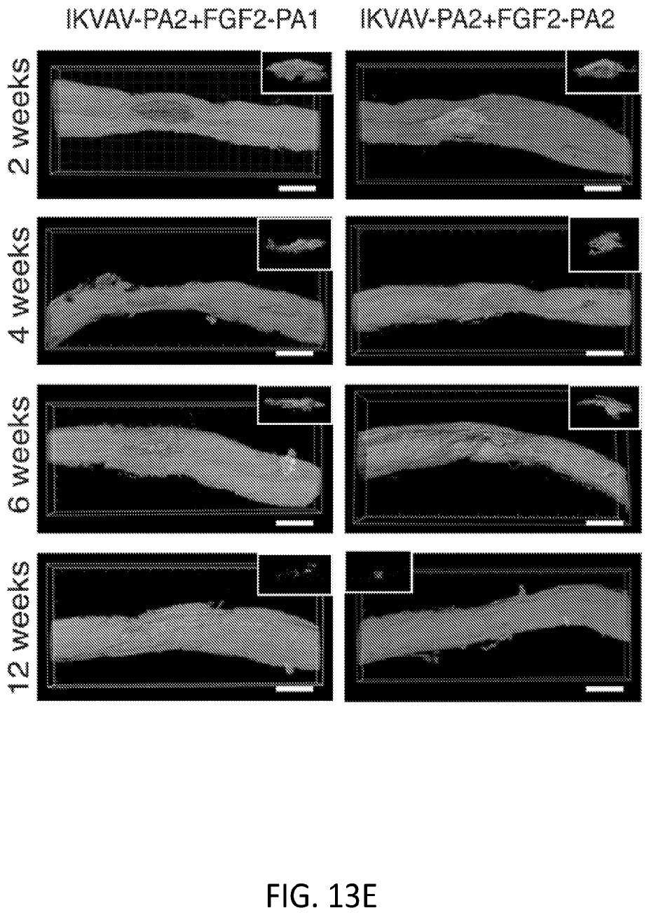

The miscible and gel-forming binary systems with similar mechanical properties, IKVAV PA2 with either FGF2 PA1 or FGF2 PA2, were taken forward to in vivo experiments (FIG. 3A and FIG. 12). Saline solutions of 90:10 molar ratio of IKVAV PA2 co-assembled with either FGF2 PA1 or with FGF2 PA2 were injected into the spinal cord of mice 24 h after a severe contusion in an established murine model of SCI (20). IKVAV PA2, which was the most bioactive single signal system, was used as a control in all in vivo experiments. All PA solutions gelled in situ when delivered into the spinal cord and localized into the damaged area. To track and quantify the bioactive scaffold's biodegradation as a function of time, the PA molecules were fluorescently labeled with Alexa 647 dye. The fluorescent materials were injected into the spinal cord 24 h post-injury and their volume was measured at 1, 2, 4, 6, and 12 weeks by fully reconstructing spinal cords using spinning disk confocal microscopy (see FIG. 3D). The soft materials biodegraded gradually within a period of 1 to 12 weeks after implantation, and no differences in biodegradation rate among the three experimental materials were observed (see FIG. 3E and FIG. 13).

Bilateral injections of biotinylated dextran amine (BDA) were administered 10 weeks after the injury into the sensorimotor cortex in order to trace the corticospinal tracts (CST), which mediate voluntary motor function (FIG. 3F). Anterogradely labeled CST axon regrowth was evaluated 12 weeks after injury in all PA and sham (injection of saline solution only) groups. This process required quantifying the number of labeled axons that regrew to the proximal lesion border and beyond. IKVAV PA1 and PA fibers lacking any bioactive signals on their surfaces (“backbone PA”) were injected as controls.

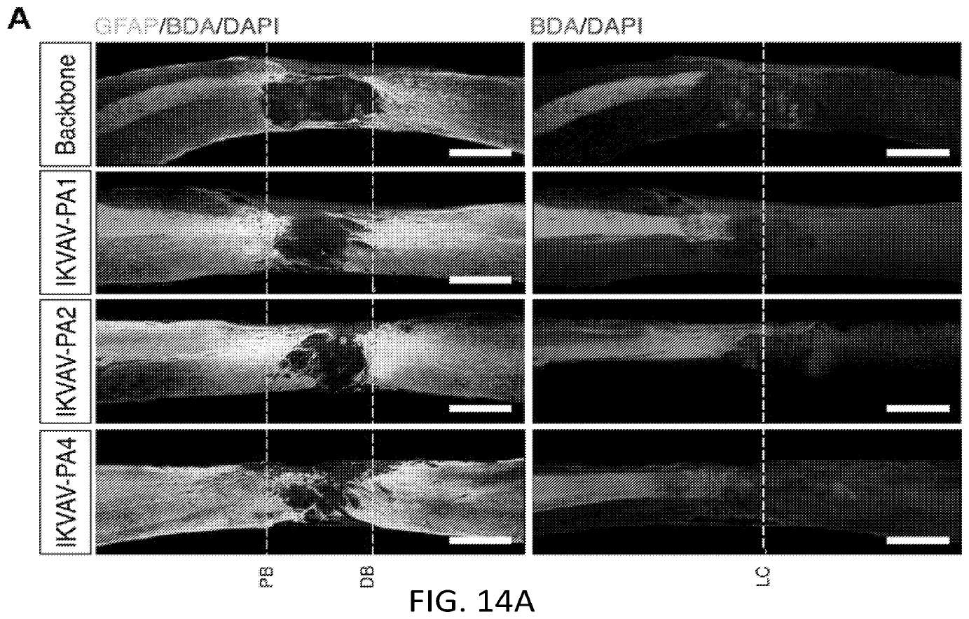

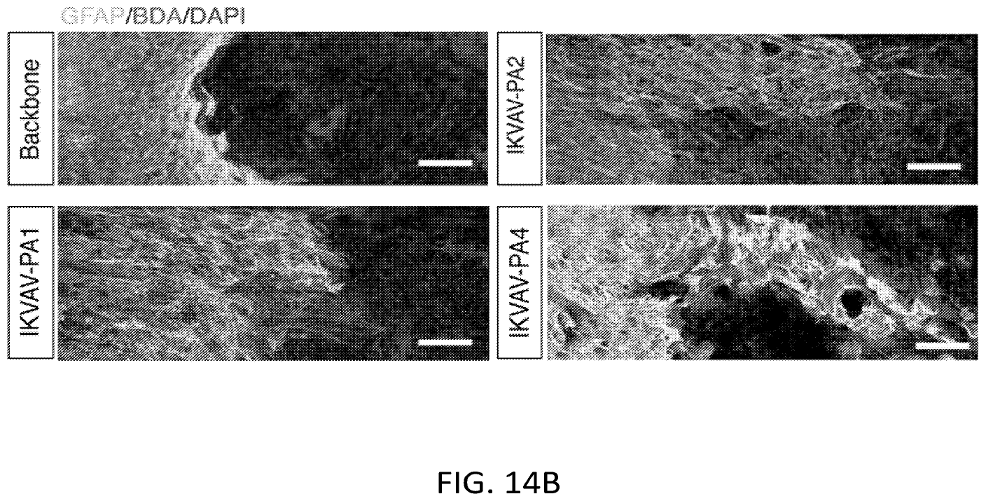

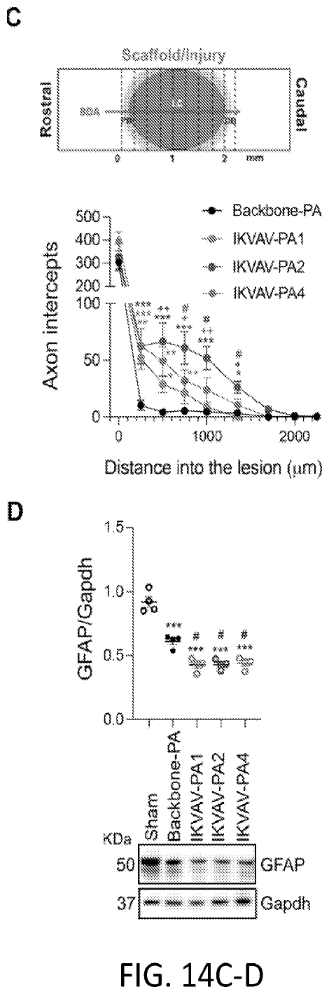

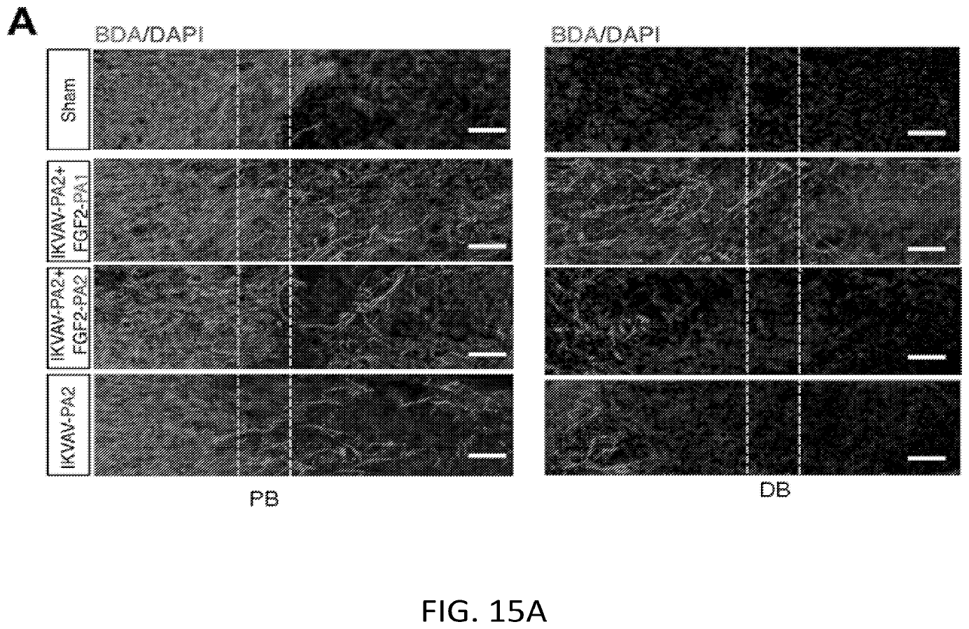

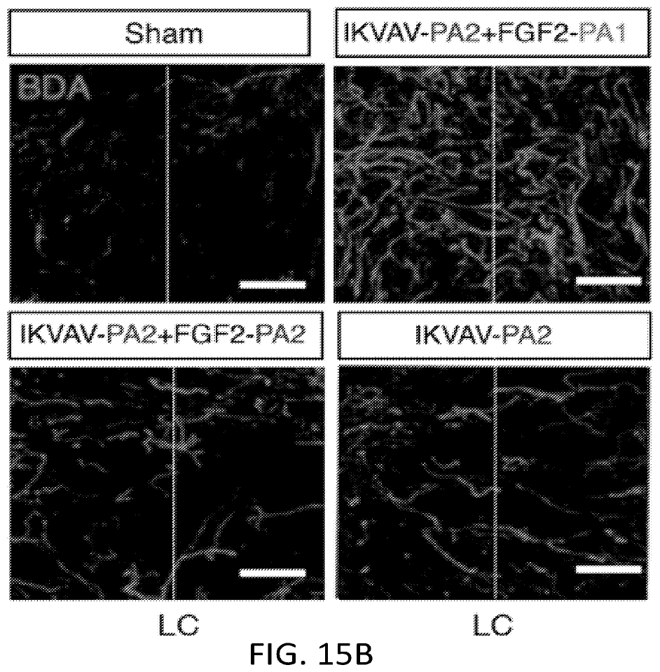

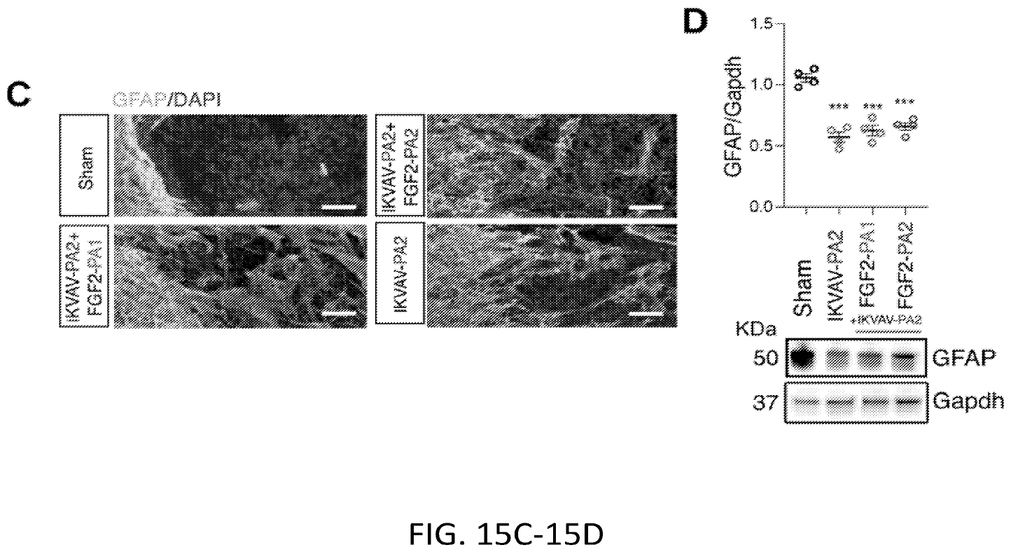

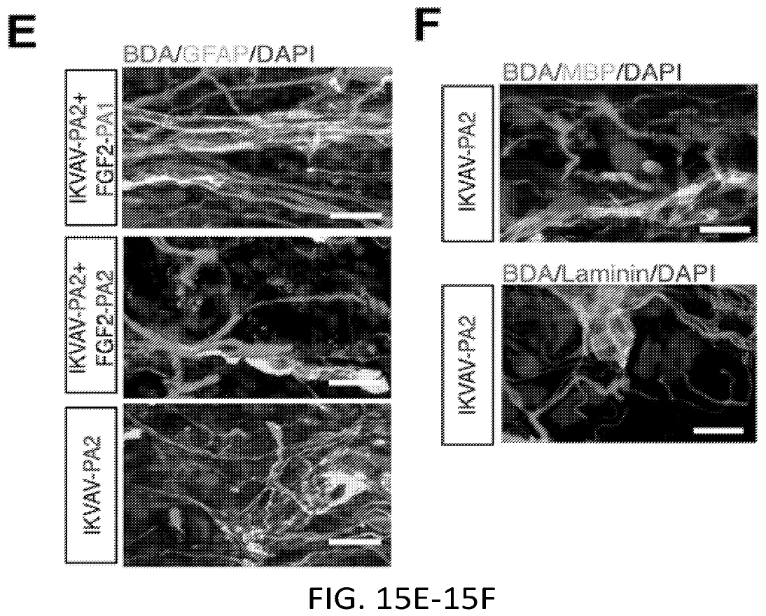

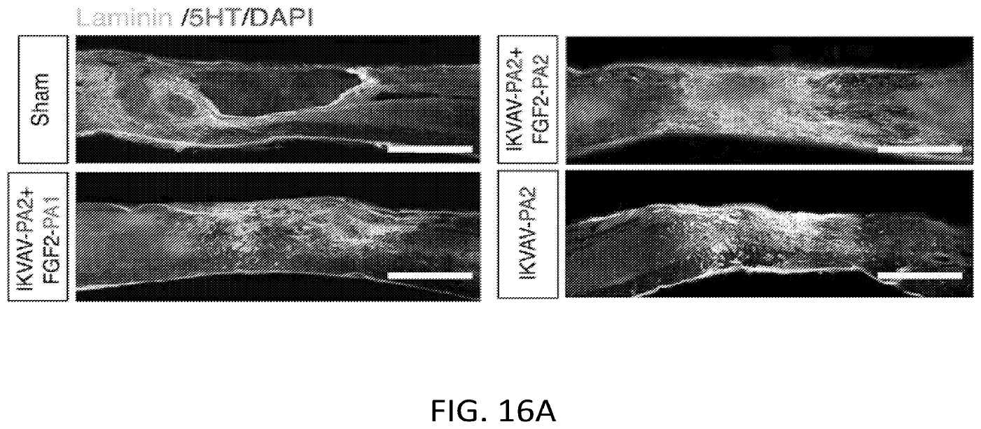

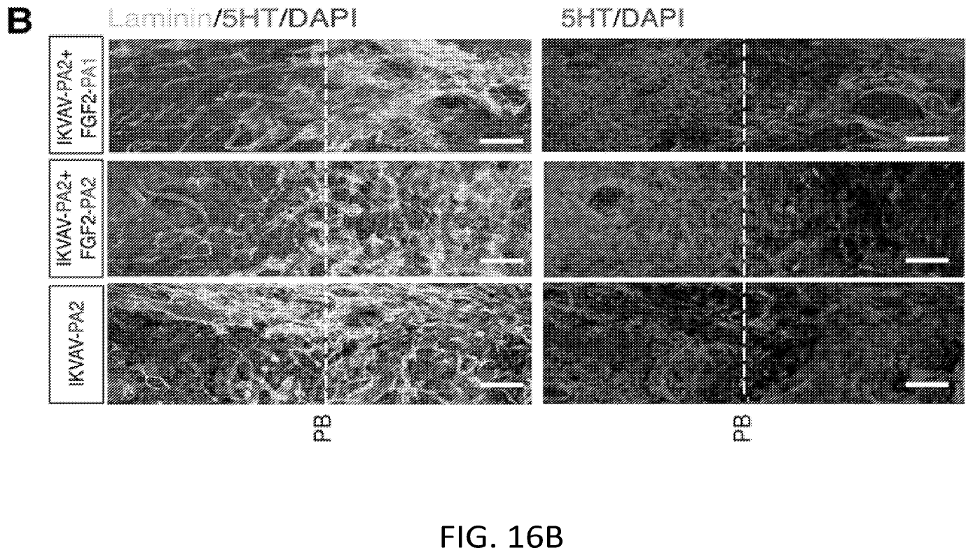

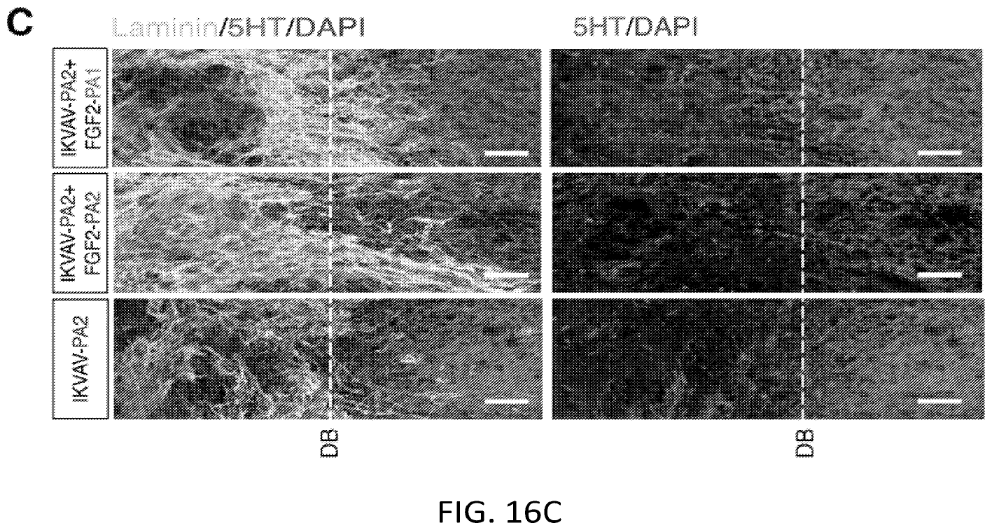

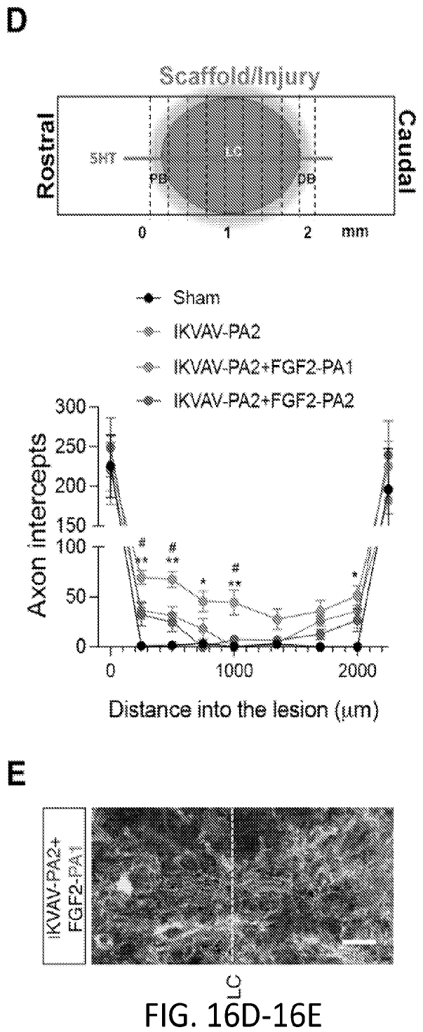

In mice injected with saline solution, regrown axons within the lesion were hardly observed, whereas some regrowth of axons for IKVAV PA1 was observed, in which fibers exhibited low mobility (FIG. 3G and FIG. 14). On the other hand, in mice injected with IKVAV PA2 alone or co-assembled with FGF2 PA2 (which shares the same A₂G₂ non-bioactive domain as IKVAV PA2), a modest, but increased axon regrowth was observed compared to the sham condition. However, injections of IKVAV PA2 co-assembled with FGF2 PA1 (which includes the V₂A₂ non-bioactive domain instead of A₂G₂) led to robust corticospinal axon regrowth across the lesion site, even surpassing its distal border (FIGS. 3, G and H, and FIG. 15). In this group, the total axon regrowth within the lesion was twofold greater than that in the group using the co-assembly of IKVAV PA2 and FGF2 PA2 and 50-fold greater than in the sham group (FIG. 3I). Serotonin axons (5HT), which may also play a role in locomotor function, also regrew within the lesion core with a similar trend as CST (FIG. 16).

Without wishing to be bound by theory, it is possible that the CST and 5HT axon regrowth observed could be in part due to the absence of a significant astrocytic scar, which is a strong barrier for axonal regeneration. In the sham and backbone PA groups, this barrier was revealed as a dense population of reactive astrocytes expressing high levels of GFAP at the borders of the injury while in all bioactive PA groups, the glial scar was less dense (FIG. 3H, FIG. 16). In agreement with these results, WB analysis showed a higher level of growth-associated protein-43 (GAP-43), which resides in the growth cone of regenerating axons, only in the most bioactive co-assembly (IKVAV PA2+FGF2 PA1) (FIG. 3J).

Next, it was determined whether PA scaffolds could induce remyelination of corticospinal axons 3 months post-injury. High levels of myelin basic protein (MBP) within the lesion were found, particularly wrapping the regrown axons in IKVAV PA2+FGF2 PA1 (FIGS. 3, J and K). Moreover, in this condition, many growing axons within the lesion were observed to be in contact with high levels of laminin and low levels of fibronectin, indicative of a reduced fibrotic core (FIGS. 3, K and L, FIG. 15). These histological and biochemical observations suggest that physical differences between the two supramolecular co-assemblies bearing two bioactive signals could greatly enhance neuro-regenerative outcomes after injury.

### SCI Model: Angiogenesis, Cell Survival and Functional Recovery

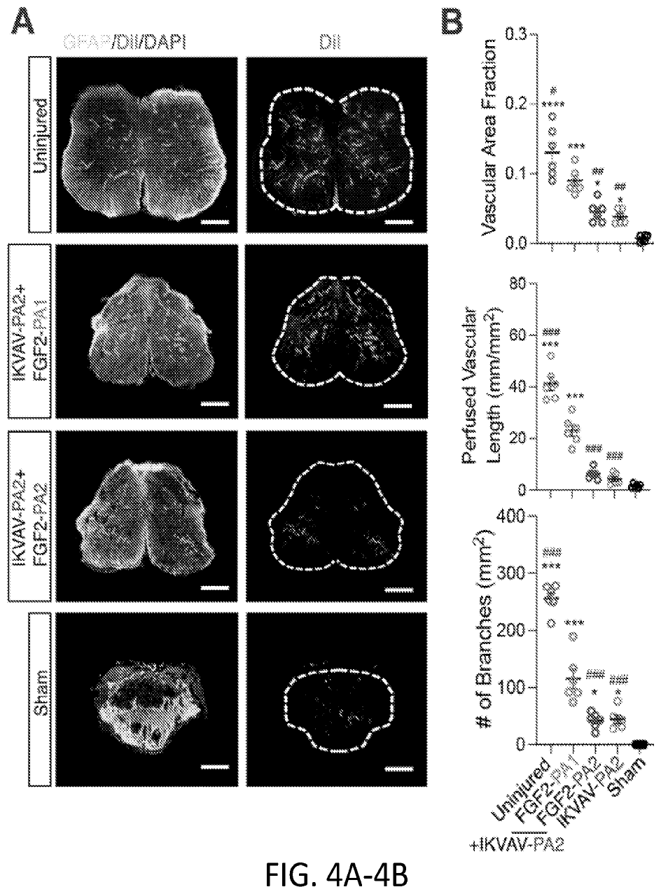

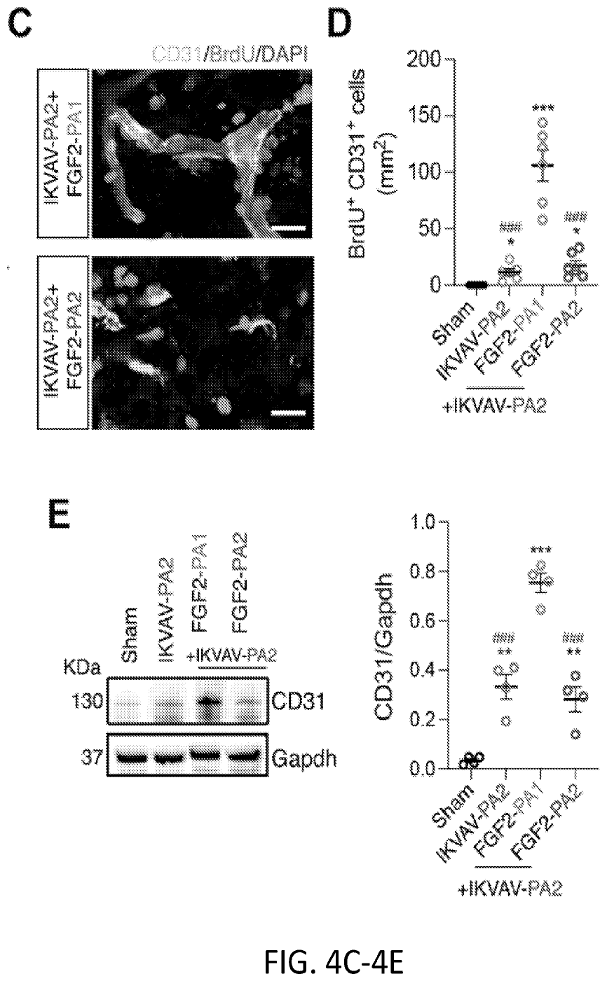

The impact of both dual signal co-assemblies on angiogenesis at the site of injury, important for a fully anatomical and functional regeneration, was next investigated. Relative to uninjured tissue sections, the transverse spinal cord sections of sham mice revealed a significant degree of tissue degeneration extending rostro-caudally more than 2.0 mm away from the center of the lesion. In this case, a significant decrease in vascular area fraction, vascular length, and branching was observed compared to the uninjured control (FIGS. 4, A and B). The existence of a functional vessel network was investigated by transcardially injecting a glucose solution containing 1,1′-dioctadecyl-3,3,3′,3′-tetramethylindocarbocyanine perchlorate (DiI), a lipophilic carbocyanine dye that incorporates into endothelial cell membranes (FIG. 4A). In groups treated with PA scaffolds, there was high preservation of the ventral tissue structure, revealing the maintenance of a functional blood vessel network. Treatment with the most bioactive co-assembly led to an increase in vascular area fraction, vascular length, and branching, especially in the dorsal region (FIGS. 4, A and B). These parameters did not differ significantly between the IKVAV PA2 alone and the less bioactive co-assembly group (IKVAV PA2+FGF2 PA2), implying that the mimetic FGF2 angiogenic signal was not functioning optimally in IKVAV PA2+FGF2 PA2.

In order to determine the origin of the blood vessels within the lesion, the thymidine analog 5′-bromo-2′-deoxyuridine (BrdU) was intraperitoneally injected during the first week post-injury. Newly formed blood vessels were observed within the lesion of the most bioactive co-assembly group 12 weeks after injury. This was confirmed by a significant increase in the number of BrdU⁺/CD31⁺ cells relative to samples for all other groups (FIGS. 4, C and D) as well as by WB analysis (FIG. 4E). The IKVAV PA2+FGF2 PA2 co-assembly and IKVAV PA2 alone led to a very modest but yet significantly increased blood vessel formation compared to the sham group.

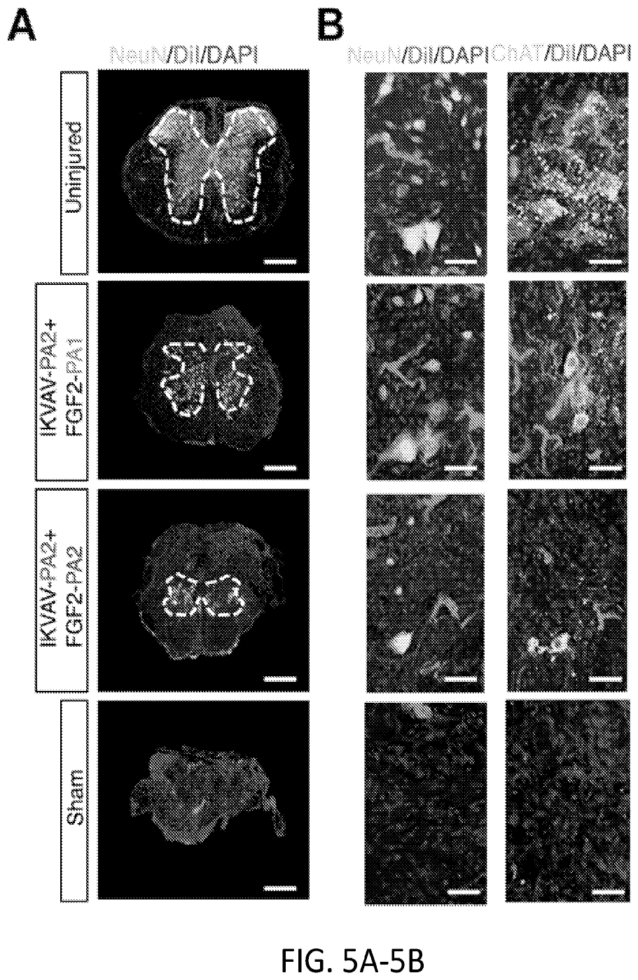

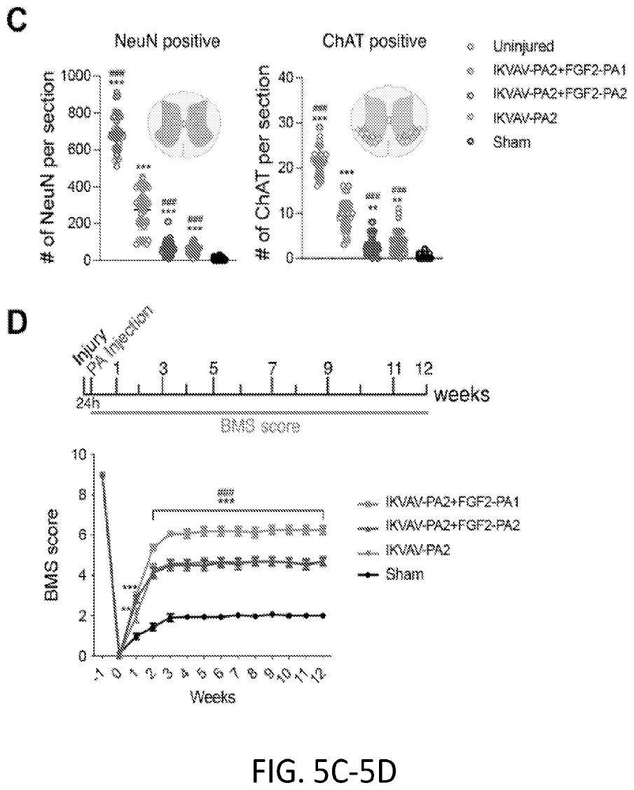

The effect of both dual signal co-assemblies on neuronal survival, maintenance of spinal circuitry and local function was also investigated. Native FGF-2 may be associated with an increase in neuronal viability after SCI. Transverse spinal cord sections of the most bioactive co-assembly group showed NeuN⁺ neurons near the newly generated vessels in the dorsal region similar to the uninjured control group (FIG. 5A). Furthermore, neurons (NeuN⁺ cells) that were also ChAT⁺ (motor neurons) were only found in the ventral horn when PAs were utilized, showing a significantly higher number in the most bioactive system relative to other groups (FIGS. 5, B and C). The lack of any double BrdU⁺/NeuN⁺ neurons within the lesion in any of the groups suggested the absence of local neurogenesis.

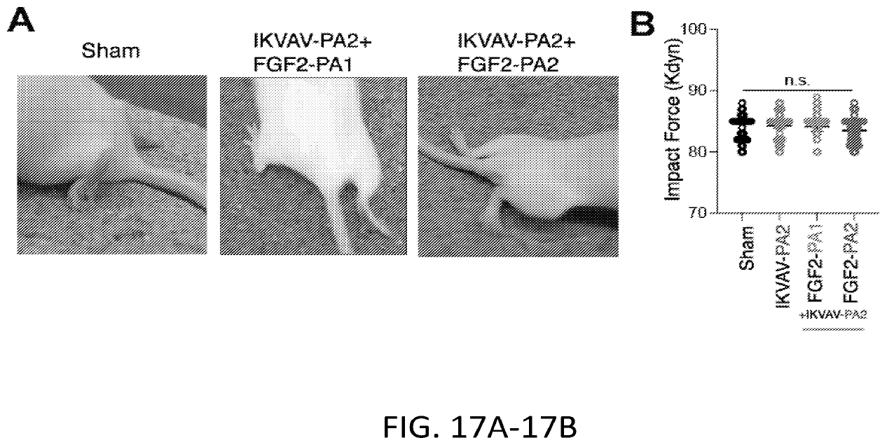

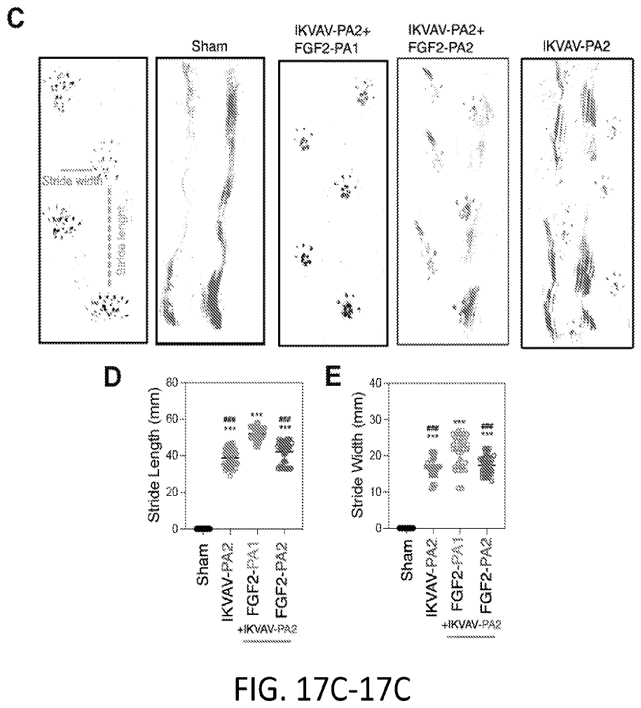

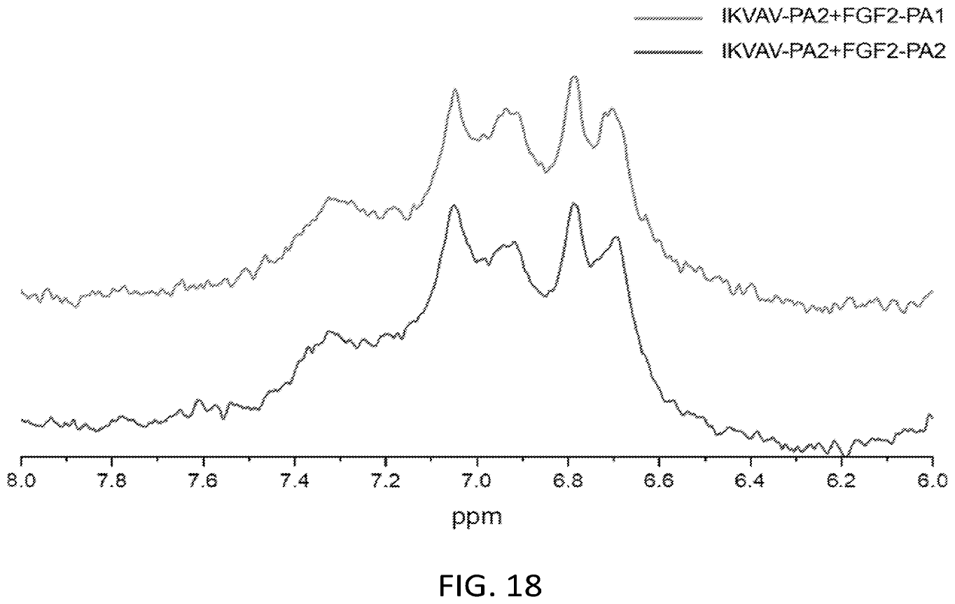

It was evaluated whether the observed axonal regeneration, angiogenesis, and local neuronal cell survival led to behavioral improvement in injured animals. For this purpose, Basso Mouse Score (BMS) open field locomotor scores and locomotor recovery by footprint analysis in all groups during the 12 weeks post-injury (FIG. 5D and FIG. 17) was evaluated. At one-week post-injury and thereafter, all PA groups demonstrated significant and sustained behavioral improvement compared to the sham group. Interestingly, three weeks post-injury, mice treated with the most bioactive co-assembly showed a significant functional recovery (5.9±0.5) compared to mice injected with IKVAV PA2+FGF2 PA2 and IKVAV PA2 alone (4.4±0.5 and 4.3±0.5, respectively) (FIG. 5D). Quantification of footprints revealed significantly larger stride length and width in mice treated with the most bioactive co-assembly relative to other groups (FIG. 17). Collectively, these data suggest that neuronal cell survival and functional recovery that was observed in dual signal systems are surprisingly linked to the differences in the chemical composition of their respective non-bioactive tetrapeptides.

### In Vitro Results on Human Endothelial and Neural Progenitor Cells

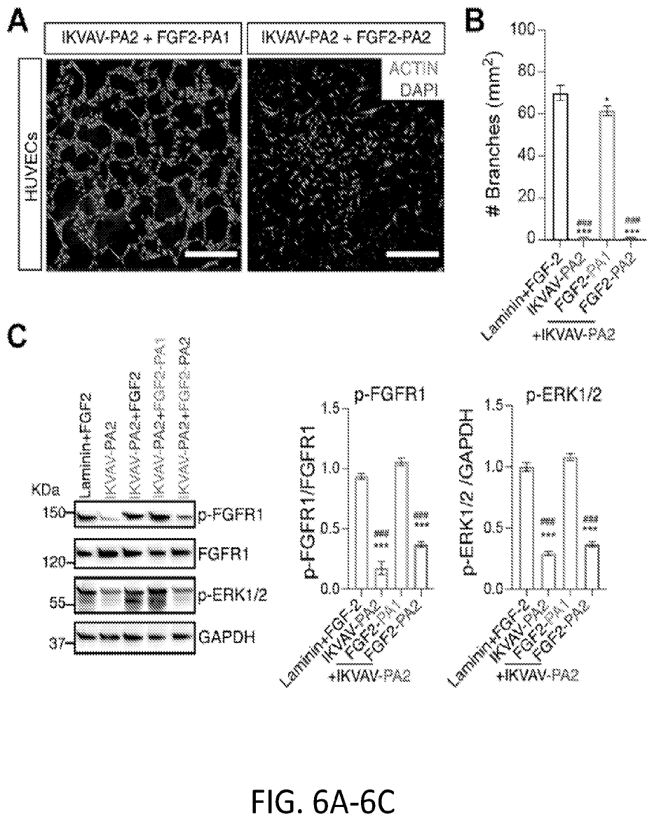

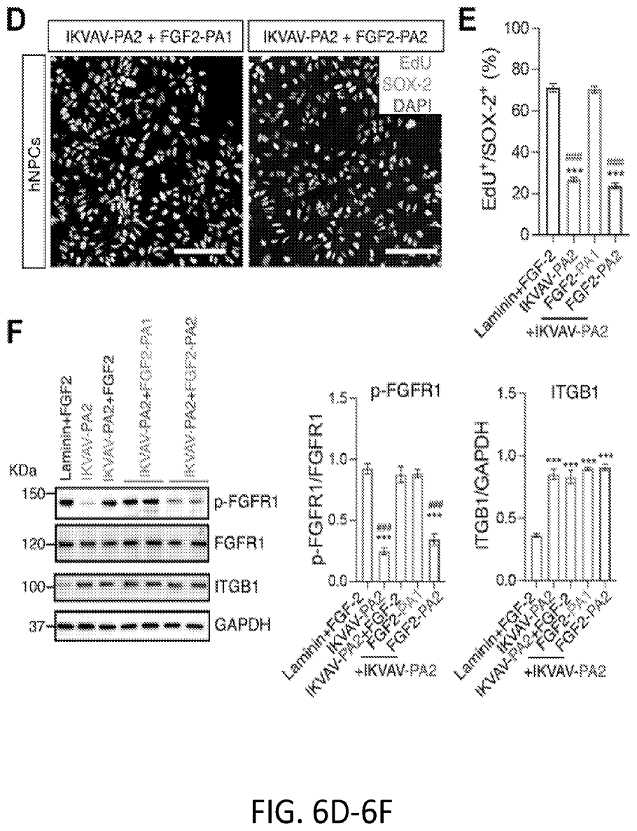

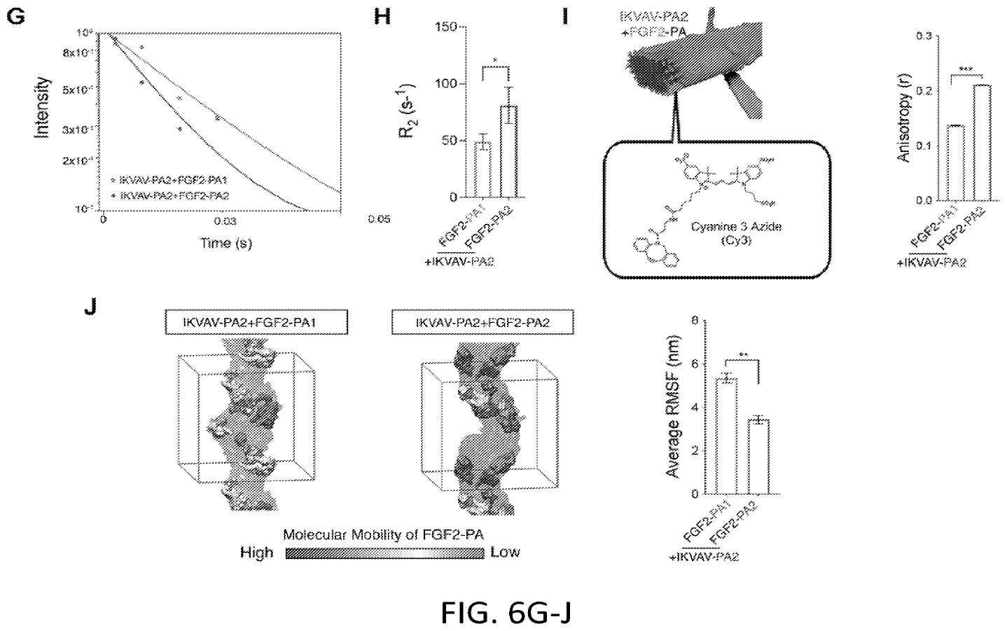

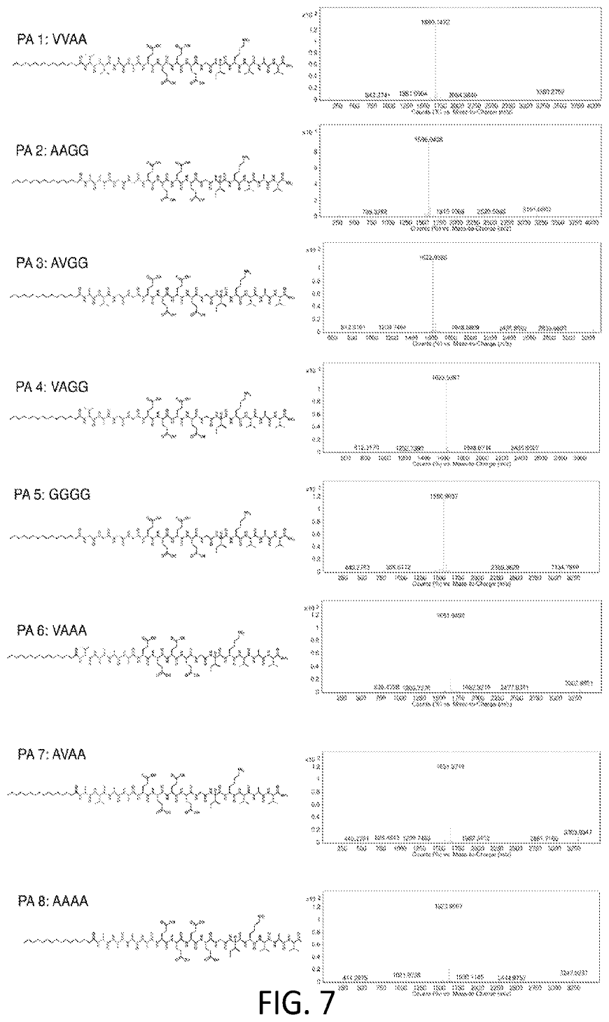

Based on results described above, the bioactivity of the FGF2 signal in vitro in both co-assemblies was evaluated using human umbilical vein vascular endothelial cells (HUVECs). Native FGF-2 enhances endothelial cell proliferation and network formation. Within 48 h of culturing HUVECs on the most bioactive co-assembly or FGF2 protein, extensive branching and formation of vessel-like capillary networks was observed (FIGS. 6, A and B). WB analysis was also performed to verify whether the observed in vitro bioactivity of the FGF2 PA1 co-assembled with the IKVAV PA2 was linked to the FGF-2 intracellular signaling pathway. HUVECs treated with the most bioactive co-assembly or native FGF-2 revealed high levels of p-FGFR1 and the downstream proteins p-ERK1/2, which activate proliferation and migration of endothelial cells (FIG. 6C). Systems containing the scrambled FGF2 mimetic sequence did not reveal any bioactivity.

To establish the simultaneous bioactivity of the IKVAV and FGF2 signals in both co-assemblies, the effects of these molecules on hNPC proliferation was assessed in vitro, by quantifying the double positive EdU⁺/SOX-2⁺ as well as the induction of ITGB1 and pFGFR-1 (FIG. 6, D to F). These experiments suggest that the FGF2 signal in the less bioactive co-assembly is largely non-functional, whereas the IKVAV signal remains operative in both. These results are consistent with observations in the SCI experiments.

### Physical Experiments and Computer Simulations on Supramolecular Motion

The potential physical reasons for the loss of in vitro and in vivo bioactivity when the tetrapeptide that follows the alkyl tail was mutated from V₂A₂ to A₂G₂ in the FGF2 PAs was investigated. Differences in dynamics between FGF2 PA molecules in the two co-assemblies were studied with T2-NMR spectroscopy and FD (FIG. 6, G to I). Relaxation rates of the aromatic protons in Y and W amino acids, which are only present in the FGF2 mimetic signal, were measured. The rates were slower in the most bioactive co-assembly, indicating greater supramolecular motion in the signaling peptide (¹H—R₂=49.3±11 s⁻¹ vs. 80.9±18.9 s⁻¹ for the less bioactive co-assembly) (FIGS. 6, G and H). FD experiments on the two co-assemblies were carried out using FGF2 PA molecules that were covalently labeled with a Cy3 dye (based on cryo-TEM images the dye did not disrupt the supramolecular assemblies). A lower anisotropy was found in the most bioactive co-assembly, indicating a higher mobility of the FGF2 signal molecules within the nanofibers (FIG. 6I).

CG-MD simulations supported the T2-NMR and FD results above by yielding higher values of RMSF for FGF2 PA molecules in the most bioactive co-assembly. The simulations also revealed that FGF2 PA molecules form clusters in both co-assemblies (slightly larger in the most bioactive system) with a distribution of mobilities (RMSF values) (FIG. 6J). The decreases in bioactivity in one of the systems could be attributed to differences in the extent of co-assembly between the two PA molecules bearing signals. However, 1D ¹H-NMR, diffusion ordered spectroscopy (DOSY), and T2-NMR of methylene units in alkyl tails indicate the occurrence of co-assembly in both systems (Table 4).

TABLE 4
Diffusion coefficients and radius of gyration of IKVAV PA2, IKVAV
PA2 + FGF2 PA1 and IKVAV PA2 + FGF2 PA2.

| Sample | D (m²/s) | Rₕ (nm) |
|---|---|---|
| IKVAV PA2 | 1.6 ± 0.01 × 10⁻¹⁰ | 1.4 |
| IKVAV PA2 + FGF2 PA1 | 8.7 ± 0.12 × 10⁻¹¹ | 2.7 |
| IKVAV PA2 + FGF2 PA2 | 8.9 ± 0.06 × 10⁻¹¹ | 2.7 |

The results obtained on greater degrees of motion in FGF2 PA1 molecules were counterintuitive because the tetrapeptide V₂A₂ (present in FGF2 PA1) had the least mobility in systems containing only IKVAV PA. The lower mobility in FGF2 PA2 molecules in the co-assembly with IKVAV PA2 was likely the result of greater interactions through hydrogen bonding and side chain contacts among the identical tetrapeptides present in both molecules. In contrast, two dissimilar tetrapeptides are present in the two molecules of the highly bioactive IKVAV PA2+FGF2 PA1 co-assembly, which would not favor a strong interaction between both types of molecules and lead to higher degrees of supramolecular motion.

The evidence for a strong interaction between IKVAV PA2 and FGF2 PA2 and less motion is the essentially invariant CD spectrum when FGF2 PA2 is added to IKVAV PA2. However, the CD spectrum was modified when the less interactive FGF2 PA1 is added to IKVAV PA2, thus suggesting a disruption of secondary structure. Thus, the greater motion detected by NMR for FGF2 PA1 molecules could indicate freer translational motion of its clusters within the fibrils or vertical motion of the signaling clusters in and out of the fibrils

### Additional Results:

### Co-Assembly Characterization

When IKVAV PA2 was co-assembled with FGF2 PAs (IKVAV PA2+FGF2 PA1 and IKVAV PA2+FGF2 PA2) both systems formed long ribbon-like structures at a molar ratio 90:10 (FIG. 3B). The morphological changes upon co-assembly were corroborated by SAXS and WAXS profiles as well as FT-IR spectra. SAXS profiles showed a slope in Guinier region of −1.2 for IKVAV PA2 and −1.6 for IKVAV PA2+FGF2 PA1 and IKVAV PA2+FGF2 PA2, indicating different degree of transition between cylindrical fibers (˜1.0) to ribbons (˜2.0). Moreover, PA co-assemblies (IKVAV PA2+FGF2 PA1 and IKVAV PA2+FGF2 PA2) showed consistent β-sheet spacing on WAXS, corroborated by the FT-IR spectra, showing similar β-sheet signature in amide I region to IKVAV PA2. High-magnification transmission electron microscopy (TEM) with negative staining of the co-assemblies showed an average fiber width of 13.7±0.3 and 15.6±0.2 nm for IKVAV PA2+FGF2 PA1 and IKVAV PA2+FGF2 PA2 respectively. The fiber width of both systems was much greater than its major counterpart alone (IKVAV PA2=10.6±0.1 nm) which could be due to the addition of FGF2 PAs to the system. The amount FGF2 PAs incorporated into the IKVAV PA2 was analyzed based on light scattering intensity (optical density) at 600 nm (O.D. 600 nm). FGF2 PAs (PA1 and PA2) at different mol % showed higher values than those same percentage co-assembled with IKVAV PA2. Therefore, although FGF2 PAs have low solubility alone, IKVAV PA2 enhances its solubility, leading to their assembly. For lower percentages of FGF2 PA (5-10 mol %) co-assembled with IKVAV PA2, the O.D. showed similar values to IKVAV PA2 alone (0 mol % of FGF2 PA) and significantly lower than FGF2 PAs free in solution.

### PA Controls in the SCI Model:

IKVAV PA1, IKVAV PA4, and PA fibers which do not have any bioactive signals on their surfaces (backbone PA) were injected as controls. In mice injected with backbone PA, regrown axons within the lesion were hardly observed, whereas some regrowth of axons for IKVAV PA1 was observed. Interestingly, IKVAV PA4, which was found to have intermediate supramolecular motion and bioactivity in vitro (between IKVAV PA1 and IKVAV PA2), also showed an intermediate level of axon regrowth in vivo.

hNPCs on Co-Assembled Systems:

hNPCs were seeded on top of the PA coatings and were found to attach and survive in a similar way for all PA conditions tested over one week. Although the number of hNPCs plated was similar for all conditions, the percentage of proliferating EdU⁺ and SOX2⁺ cells increased when hNPCS were cultured on IKVAV PA2+FGF2 PA1 or with native FGF-2, whereas that percentage decreased significantly when cells were cultured on IKVAV PA2+FGF2 PA2, IKVAV PA2 alone or commercial laminin (FIG. 6E). One week later, IKVAV PA2+FGF2 PA1 maintained a high pool of SOX2⁺ cells to the same extent as the native FGF-2 protein. Conversely, cells plated on the IKVAV PA2+FGF2 PA2, IKVAV PA2 and laminin resulted in an increased percentage of neuronal progenitor cells PAX6⁺ indicative of more differentiated cells. It was next examined whether the observed bioactivity of IKVAV PA2+FGF2 PA1 is indeed linked to the ITGB1 and FGFR-1 respectively by following their expression by WB. P-FGFR-1 was highly expressed in hNPCs seeded on IKVAV PA2+FGF2 PA1 or treated with native FGF-2, while active ITGB1 exhibited significantly higher levels in cells cultured on all PA conditions containing IKVAV PA2. Differences in the phenotypic profiles between cells plated on IKVAV PA2 co-assembled with FGF2 PA1 and those plated on IKVAV PA2 co-assembled with FGF2 PA2 were also observed. IKVAV PA2+FGF2 PA1 triggered higher expression of the neural stem cell marker SOX-2, neuronal marker β-Tubulin-III (TUJ-1), and the postmitotic marker PH3 in a similar way to cells seeded on laminin coatings treated with native FGF-2.

### ¹H-NMR, DOSY and Relaxation NMR for Co-Assembly Systems

¹H-NMR spectra revealed broader and lower intensity peaks from methylene protons in both co-assemblies relative to IKVAV PA2 alone and DOSY confirmed these results. Proton relaxation rate data was also obtained using T2-NMR much higher values for the co-assemblies vs. IKVAV PA2 (IKVAV PA2+FGF2 PA1: ¹H—R₂=59.6±1.9 s⁻¹ and IKVAV PA2+FGF2 PA2: ¹H—R₂=52.7+1.7 s⁻¹, and IKVAV PA2: ¹H—R₂=6.4 i 0.1 s⁻¹) were found. These results indicate that co-assembly of both signals does occur in both systems.

### Discussion

Described herein are bioactive scaffolds which physically and computationally reveal greater supramolecular motion, thus leading to greater functional recovery from SCI in a murine model. In one-dimensional scaffolds of non-covalently polymerized bioactive molecules, it was expected that polyvalency effects would help cluster receptors for effective signaling. It was also expected that the internal structure of the supramolecular scaffolds could limit free motion and favorably orient signals toward receptors perpendicular to their fibrillar axis. However, the surprising finding in this work is that the intensity of molecular motions within the bioactive fibrils, as measured on the bench, correlated with enhanced axonal regrowth, neuronal survival, blood vessel regeneration, and functional recovery from SCI. Computer simulations and experimental data suggest that translation on the scale of nanometers within or vertically out of the assemblies to reach receptor sites enhances bioactivity. Without wishing to be bound by theory, a highly agile and physically plastic supramolecular scaffold could be more effective at signaling receptors in cell membranes undergoing rapid shape fluctuations. An alternative explanation for the cause of the recovery could be broadly more favorable interactions of the molecularly dynamic scaffolds with the protein milieu of the ECM. In sum, the scaffolds described herein provide great opportunities in the structural design of dynamics to optimize the bioactivity of therapeutic supramolecular polymers.

### Materials and Methods

### Characterization of Peptide Amphiphile Nanostructures:

Synthesis and Purification PA synthesis and preparation: IKVAV PAs (IKVAV PA1: C₁₆-VVAAEEEEGIKVAV (SEQ ID NO: 20) and IKVAV PA2: C₁₆-AAGGEEEEGIKVAV (SEQ ID NO: 12), IKVAV PA3: C₁₆-AVGGEEEEGIKVAV (SEQ ID NO: 21), IKVAV PA4: C₁₆. C₁₆-VAGGEEEEGIKVAV (SEQ ID NO: 22), IKVAV PA5: C₁₆-AAAAEEEEGIKVAV (SEQ ID NO: 25), IKVAV PA6: C₁₆-GGGGEEEEGIKVAV (SEQ ID NO: 18), IKVAV PA7: C₁₆-VAAAEEEEGIKVAV (SEQ ID NO: 23), IKVAV PA8: C₁₆-AVAAEEEEGIKVAV (SEQ ID NO: 24), FGF2 PAs (FGF2 PA1: C₁₆-VVAAEEEEGYRSRKYSSWYVALKR (SEQ ID NO: 13) and FGF2 PA2: C₁₆-AAGGEEEEGYRSRKYSSWYVALKR (SEQ ID NO: 14), their scrambled versions scr-IKVAV PAs (VVIAK PA1: C₁₆-VVAAEEEEGVVIAK (SEQ ID NO: 27) and VVIAK PA2: C₁₆-AAGGEEEEGVVIAK (SEQ ID NO: 28)) and scr-FGF2 PAs (scr-FGF2 PA1: C₁₆-VVAAEEEEGWRSKKYSLYYVASRR (SEQ ID NO: 29) and scr-FGF2 PA2: C₁₆-AAGGEEEEGWRSKKYSLYYVASRR (SEQ ID NO: 30)), and the backbone PA (C₁₆-VVAAEE (SEQ ID NO: 19)) (see Table 5 for list of material names and corresponding peptide sequences; C₁₆ is a palmitoyl group) were synthesized by standard fluorenylmethoxycarbonyl (Fmoc) solid-phase peptide synthesis using a CEM model Liberty Blue Microwave Assisted Peptide Synthesizer on rink amide MBHA resin. Automated coupling reactions were performed using 4 equiv. Fmoc-protected amino acid, 4 equiv. of N,N′-diisopropylcarbodiimide (DIC), and 8 equiv. ethyl(hydroxyimino)cyanoacetate (Oxyma pure).

Removal of the Fmoc groups was achieved with 20% 4-methylpiperidine in DMF. Peptides were cleaved from the resin using standard solutions of 95% TFA, 2.5% water, 2.5% triisopropylsilane (TIS) and precipitated with cold ether. For IKVAV PAs, their scrambled versions, and the backbone PA, basic purification using reverse-phase high-performance liquid chromatography (HPLC) was then performed using a Phenomenex Gemini NX-C18 column, (C18 stationary phase, 5 um, 110 Å pore size, 150'30 mm) on a Shimadzu model Prominence modular HPLC system, two LC-20AP solvent delivery units, SPD-M20 Å diode array detector and a FRC-10A fraction collector, using H₂O/CH₃CN gradient containing 0.1% NH₄OH (v/v) as an eluent at a flow rate of 25.0 mL/min. FGF2 PAs and their scrambled versions were purified under acidic conditions (0.1% TFA v/v in the water and CH₃CN) using a Phenomenex Kinetex C8 column, (C8 stationary phase, 5 m, 100 Å pore size, 150×30 mm). The purity of lyophilized PAs was analyzed by liquid chromatography-mass spectrometry (LC-MS) using a Phenomenex Jupiter 4 m Proteo 90 Å column (C12 stationary phase, 4 m, 90 Å pore size, 1×150 mm) or Phenomenex Gemini C18, (C18 stationary phase, 5 m, 110 Å pore size, 150'1 mm) on an Agilent model 1200 Infinity Series binary LC gradient system, using H₂O/CH₃CN gradient containing 0.1% formic acid or NH₄OH (v/v) as eluents, respectively, with a flow rate of 50 μL/min. Electrospray ionization mass (ESI-mass) spectrometry was performed in positive scan mode on an Agilent model 6510 Quadrupole Time-of-Flight LC-MS.

For PAs with a covalently linked dye, an Alexa Fluor®-647 labeled-IKVAV PA2 and Cy3-labeled-FGF2 PAs, were synthesized with an added cysteine or azidolysine on the C-terminus of the sequences above respectively. The purified IKVAV PAs were dissolved with tris(2-carboxyethyl) phosphine (TCEP) hydrochloride (5 equiv. with respect to the PA) in pH 8 Tris buffer and reacted with maleimide-functionalized Alexa Fluor®-647 (Thermo Fisher). Purified FGF2 PAs were dissolved in N,N-dimethylformamide (DMF) and reacted with dibenzocyclooctyne-functionalized Cy3-DBCO (Click Chemistry Tools). The final dye-labeled PA products were purified by HPLC, lyophilized, and stored until use.

### Co-Assembled PA Preparation

For in vitro experiments: After lyophilization, PA powder was reconstituted in 150 mM NaCl and 3 mM KCl solution and adjusted to a pH of 7.4 using 1 μL additions of 1 M NaOH to ensure cell compatibility and material consistency. Different bioactive PAs were mixed at different mol % (see Table 3) and horn sonicated at 10% intensity, three times for 10 seconds. PA solutions were annealed at 80° C. for 30 min and then slowly cooled at 1° C. per minute to reach a final temperature of 27° C. using a thermocycler (Eppendorf PCR Thermocycler) for even and controlled heating and cooling of all samples. To prepare a PA coated substrate, 24-, 12-, or 6-well polystyrene cell culture plate or 12 mm and 18 mm glass coverslips (German Glass, Chemglass Life Science) were coated with poly-D-lysine (0.01 mg/mL, Sigma-Aldrich) for 3 h at 37° C. The plates were then rinsed with MilliQ water three times and allowed to dry for 4 h. PAs were painted on the coverslips or tissue culture plates by dragging a pipette (8-30 μL of annealed PAs (1 wt %)) to extrude a thin, even coating of material across the surface. PA coatings were incubated for 3 h at room temperature. The plates were gently rinsed with media before further use. For dye-labeled PAs experiments, Cy3-labeled-FGF2 PAs were co-assembled at 1 mol % with their corresponding non-labeled PA counterparts (see table 3).

For in vivo experiments: After lyophilization, PA powder was reconstituted in sterile Isotonic Saline Sodium Chloride, 0.9% (w/v) (Ricca Chemical) at a concentration of 1 mg/100 μl. The resulting PA solution was then adjusted to a pH of 7.4 using 1 μL additions of sterile 1 M NaOH, followed by co-assembly of IKVAV PA2 with 10 mol % FGF2 PA1 or FGF2 PA2 (see table 3). After mixing, the solutions were sonicated and annealed. For dye-labeled PA experiments, Alexa-647 labeled-IKVAV PA2 was co-assembled at 1 mol % with their corresponding non-labeled PA counterparts (see table 3).

### Transmission Electron Microscopy (TEM)

To prepare the negatively stained TEM specimen, 7 μL of PA sample solution (1 wt % PA prepared in 150 mM NaCl and 3 mM diluted 10 times in MilliQ water) was applied on the shiny side of a glow discharged grid (300 mesh copper with carbon film, Electron Microscopy Sciences), followed by rinsing three times with MilliQ water to remove the excess of salt. The grid was then stained with filtered 2 wt % uranyl acetate aqueous solution and dried in air. TEM imaging was carried out on a JEOL 1230 microscope with a LaB6 filament at 100 kV accelerating voltage, equipped with a Gatan 831 CCD camera. A cold finger was introduced for sample stabilization during imaging.

### Cryogenic Transmission Electron Microscopy (Cryo-TEM)

Plunge-freezing for Cryo-TEM samples were prepared using a FEI model Vitrobot MarkIV (FEI). 7 μL of the sample solution ([PA]=0.05-0.1 wt % in H₂O) was deposited on a plasma-cleaned TEM grid (300 mesh copper with lacey carbon, Electron Microscopy Sciences) and held in place with tweezers mounted on the Vitrobot. The specimen was blotted in an environment with 100% humidity at room temperature (blot force: 3, blot total: 1-2, wait time: 0.5-1 s, blot time: 3 s, drain time: 0-1 s), and plunged into a liquid ethane reservoir cooled by liquid nitrogen. The vitrified samples were stored in liquid nitrogen and then transferred to a Gatan 626 Cryo-TEM holder. Cryo-TEM images were obtained using a JEOL1230 electron microscope operating with a LaB6 filament at an accelerating voltage of 100 kV, equipped with a Gatan 831 CCD camera.

### Scanning Electron Microscopy (SEM)

PA coatings with or without cells were fixed in a mixture of paraformaldehyde (2.0%, Electron Microscopy Sciences), glutaraldehyde (2.5%, Electron Microscopy Sciences) in phosphate buffered saline (1×, Gibco) for 20 min. The fixative was removed, and the water was exchanged with ethanol by incubating the samples in a gradation of ethanol solutions with increasing concentration (30-100%) of 200 Proof ethanol (Decon Laboratories, Inc). Critical point drying (Tousimis Samdri-795) was used to remove the excess water. A purge cycle of 20 min was used to ensure sufficient exchange occurred. The resulting dehydrated samples were mounted on stubs using carbon adhesive tape (Electron Microscopy Sciences) and in some cases, carbon glue (Electron Microscopy Sciences). Samples were coated with approximately 10 nm of osmium (Filgen, OPC-60A) to make the sample surface conductive for imaging. All images were taken with an accelerating voltage of 2 kV with a Hitachi SU8030 SEM instrument.

### X-Ray Scattering

Small-angle X-ray scattering (SAXS), medium-angle X-ray scattering (MAXS), a wide-angle X-ray scattering (WAXS) experiments were performed at beamline 5-ID-D of the DuPont-Northwestern-Dow Collaborative Access Team (DND-CAT) Synchrotron Research Center at the Advanced Photon Source, Argonne National Laboratory. 100-150 μL of sample solution ([PA]=1-3 w/v % (˜6-10 mM) in aqueous NaCl and KCl ([NaCl]=150 mM and [KCl]=3 mM)) was introduced into capillary flow-cell with a fixed diameter and irradiated with X-ray for an exposure time of 0.5, 2, 3, or 10 s. Samples were oscillated at a rate of 10 L/sec in the capillary with a syringe pump during sample measurement to prevent damage due to beam overexposure. Data was collected with an X-ray energy at 17 keV (l=0.83 Å) with a triple-area detector system. The scattering intensity was recorded in the interval 0.002390<q<4.4578 Å⁻¹. The wave vector q is defined as =(4π/λ) sin(θ/2), where θ is the scattering angle. The acquired 2D scattering data were then reduced to 1D intensity vs. wavevector plots via azimuthal integration around the beam center in GSAS-II software. Background scattering patterns were obtained from samples containing 150 mM NaCl and 3 mM KCl. This background data was then subtracted from experimental data. All data was analyzed using the Irena software package running on IgorPro software.

### Circular Dichroism (CD) Spectroscopy

Each PA sample was diluted to concentrations between 0.01-0.04 wt % in either H₂O (no salt samples) or buffer containing 150 mM NaCl and 3 mM KCl (high salt). CD spectra was recorded on a JASCO model J-815 spectropolarimeter using a quartz cell of 0.5 mm optical path length. Continuous scanning mode was used with a scanning speed of 100 nm per minute with the sensitivity set to standard mode. High Tension (HT) voltage was recorded for each sample to ensure that the measurement was not saturated. An accumulation of three measurements was used and a buffer sample was background-subtracted to obtain final spectra. The final spectra were normalized to final concentration of each sample using a molar averaged molecular weight.

### Fourier Transformed Infrared (FT-IR) Spectroscopy

FT-IR spectra were recorded on a Bruker Tensor 37 FT-IR spectrometer. Samples were prepared in deuterated water (D₂O) and placed between two CaF₂ windows with a spacing of 50 m. Final spectra are the result of 25 scans with 1 cm⁻¹ resolution and atmospheric CO₂ and H₂O were background subtracted.

### Fluorescence Depolarization

An aqueous solution of 100 μL of annealed PA ([PA]=6 mM, [KCl]=3 mM, [NaCl]=150 mM) was added a THF solution (2 μL) of 1,6-diphenyl-1,3,5-hexatriene (DPH; 1.4 mM), and the mixture was incubated for 30 min at 25° C. Then, the mixture was diluted with aqueous KCl and NaCl (1900 μL, [KCl]=3 mM, [NaCl]=150 mM), incubated for 10-30 min at 25° C. to afford a solution of DPH (1.4 μM)-embedded PA (300 μM). To obtain IKVAV PA2 (A₂G₂) or IKVAV PA5 (G₄) in the presence of CaCl₂), 5 mM CaCl₂) was then added to the DPH-embedded PA aqueous solution to afford PA: CaCl₂) at a molar ratio of 6:1. DPH was exited at 336 nm and emission was recorded at 450 nm on an ISS model PC1 spectrofluorometer with a 300 W xenon arc lamp with power of 18 A. Excitation slit and emission slit widths were set as 1 mm (8 nm bandwidth). Anisotropy was calculated using the following equation:

$$A = \frac{{F}_{‖} - {gF}_{⊥}}{{F}_{‖} + 2 {gF}_{⊥}}$$

Where F_{∥} represents the parallel intensity to the excitation plane, F_{⊥} is the perpendicular intensity to the excitation plane, g is grating factor (G-factor) that represents the intensity ratio of the sensitivity of the detection system for vertically and horizontally polarized light. G-factors were determined individually in each measurement. Results were averaged based on 34 iterations from two measurements.

### Cy3 Functionalized FGF2 PA Samples for Fluorescence Studies

PA aqueous solutions ([PA]ₜₒₜₐₗ=6 mM, [KCl]=3 mM, [NaCl]=150 mM) of 90:10 molar ratio IKVAV/FGF2 mixture was prepared with 1 mol % of the FGF2 PAs functionalized with Cy3 DBCO dye. 100 μL of this solution was diluted with aqueous KCl and NaCl (1900 μL, [KCl]=3 mM, [NaCl]=150 mM) and the system was incubated for 10 min at 25° C. to equilibrate. Final PA and dye concentrations were 300 μM and 0.3 μM, respectively. Cy3 was exited at 535 nm and emission was recorded at 575 nm.

### Rheology

PA materials were prepared using the method described above for in vitro studies. An MCR302 Rheometer (Anton Paar) was used for all rheological studies. The instrument stage was set to 37° C. to simulate in vitro and in vivo conditions. The PA solution (150 μL) was placed on the sample stage and 30 μL of 25 mM CaCl₂) solution was pipetted onto the underside of a 25 mm cone plate positioned above the material. The plate was slowly lowered to the measuring position and a humidity collar was used to enclose the sample plunger and prevent sample evaporation during each 45 min experimental run. For each experiment, the sample was equilibrated for 30 minutes with a constant angular frequency of 10 [rad/s] and 0.1% strain. The storage and loss modulus (G′ and G″) were recorded after a plateau occurred.

### Optical Density (O.D.)

PA materials were prepared using the method described above for in vitro studies. The PA solutions were further diluted with a 1× saline solution to a total volume of 300 μL. 100 μL of these suspensions was pipetted into triplicate wells of a 96 well plate and their optical density was recorded at 600 nm using a Cytation3 cell imaging multi-mode reader (BioTek).

### Immobilization of IKVAV Peptide on Glass Surface

Borosilicate glass coverslips (12 mm in diameter; Fisher Scientific) were modified with synthetic IKVAV peptide. Borosilicate glass coverslips were cleaned with 2% (v/v) micro-90 detergent (Sigma-Aldrich) for 30 min at 60° C., rinsed six times with distilled water, rinsed with ethanol and then dried. Coverslips were plasma-etched (Harrick Plasma PDC-001-HP) with 02 for 30 sec, then immediately incubated in a 2% (v/v) solution of (3-aminopropyl) triethoxysilane (Sigma-Aldrich) in ethanol for 15 min. Coverslips were then rinsed twice with ethanol and twice with water and then dried in the oven. IKVAV peptide was then prepared at 50 nmol/mL in a 1.25 mg/mL solution of 1-ethyl-3-(dimethylaminopropyl) carbodiimide (Acros Organics) with 2% DMF (Sigma-Aldrich). Coverslips were incubated with this solution for 3.5 h at 40° C. After incubation, coverslips were washed with 100% acetic anhydride (Fisher Chemical), 2 M hydrochloric acid (Fisher Chemical), and 0.2 M sodium bicarbonate in succession. After rinsing with excess water, samples were sonicated in 4 M urea for 10 min followed by 1 M NaCl for 10 min and then rinsed with an excess amount of water and dried at 100° C. for 1 h.

### NMR Experiments

NMR spectra were acquired at 600 MHz on a Tecmag NMR spectrometer using a Doty diffusion probe with a sweep width of 6 kHz and 16k data points or at 600 MHz on a Brucker Neo system with QCI-F cryoprobe.

NMR spectra for IKVAV PAs were recorded at 25° C. using TFA-d, H₂O/D₂O in 9/1 ratio (D₂O contains 0.05 wt. % 3-(trimethylsilyl) propionic-2,2,3,3-d₄ acid, sodium salt) as solvents. Chemical shifts are reported in part per million (ppm). Structural assignment was performed using ¹H,¹H-gCOSY, ¹H,¹³C-gHSCQAD, TOCSY and NOESY. Multiplicities are quoted as singlet (s), doublet (d), multiplets (m), doublet of doublets (dd), doublet of doublet of doublets (ddd), triplet (t), quartet (q). The 900 pulse width was 15 s and typical spectra required 32 scans. Additional scans (512) were required for accurate estimation of the aromatic signal intensity since the epitope containing aromatic protons was present at only 10 mol %.

The diffusion coefficients were measured by pulse-field gradient NMR using the longitudinal eddy-current delay with bipolar pulse pairs pulse sequence with a maximum gradient strength of 53.5 G/cm and 16 values for the gradient strength. The peak intensity I was measured and fit to the Stejskal-Tanner equation:

$$I = {I}_{0} {e}^{- ( D {γ}^{2} {g}^{2} {δ}^{2} ( Δ - \frac{δ}{3} )} )$$

where I₀ is the intensity in the absence of the gradient pulse, D is the diffusion coefficient, γ is the proton gyromagnetic ratio, g is the gradient strength, δ is the length of the pulse field gradient pulse (2 ms) and Δ is the diffusion delay (0.1 s).

The radius of gyration R_{g} was calculated from the Stokes-Einstein equation as:

$${R}_{g} = \frac{{k}_{B} T}{6 π η D}$$

where k_{B} is the Boltzmann constant, T is the temperature, and f is the viscosity.

The spin-spin relaxation rates were measured using the Carr-Purcell-Gill-Meiboom pulse sequence with a delay time of 0.2 ms in a variable loop. The peak intensity data was fit to and exponential in the form:

$$I = {I}_{0} {e}^{- ( {R}_{2} τ )} + b$$

where τ is the length of the delay time, R2 is the spin-spin relaxation rate and b are the baseline.

### Peak Assignments

IKVAV PA1: ¹H-NMR (600 MHz, TFA-d₁): δ 0.83 (t, 3H, J=6.6 Hz, Pal-CH₃), 0.88 (t, 3H, J=7.2 Hz, Ile-C_{(δ)}H₃), 0.92-1.01 (m, 24H, 4× Val-C_{(γ)}H₃), 1.25 (m, 24H, several Pal-CH₂), 1.49 (m, 9H, 3× Ala-C_{(β)}H₃), 1.67 (m, 8H, 4× Glu-C_{(β)}H₂), 2.1 (m, 8H, 4× Glu-C_{(γ)}H₂), 2.24 (m, 3H, Pal-C_{(α)}H₂), 2.59 (m, 3H, 3× Val-C_{(β)}H), 2.81 (t, 2H, J=7.2 Hz, Lys-C_{(ε)}—H₂), 3.88 (m, Lys-C_{(α)}H₂, Ile-C_{(α)}H₂), 3.97 (m, 4H, 4× Glu-C^{(α)}H₂), 4.01 (m, 3H, 3× Val-C_{(α)}H), 4.38-4.5 (m, 6H, Gly-C_{(α)}H₂, 3×Ala-C_{(α)}H, Val-C_{(α)}H), 4.62-4.75 (m, Ala-C_{(α)}H₂).

IKVAV PA2: ¹H-NMR (600 MHz, TFA-d₁): δ 0.80 (t, 3H, J=6.5 Hz, Pal-CH₃), 0.86 (t, 3H, J=7.4 Hz, Ile-C_{(δ)}H₃), 0.92-0.96 (m, 12H, 2× Val-C_{(γ)}H₃), 1.01 (dd, J=10.9 Hz, 6.7 Hz, Ile-C_{(γ)}H₃), 1.21-1.33 (m, 24H, several Pal-CH₂), 1.45 (d, J=7.1 Hz, Ile-C_{(β)}H₃), 1.49 (m, 9H, 3×Ala-C_{(β)}H₃), 1.66 (m, 2H, Lys-C_{(β)}H₂), 1.72 (m, 2H, Lys-C_{(γ)}H₂), 1.85 (m, 8H, 4×Glu-C_{(β)}H₂), 2.1 (m, 8H, 4× Glu-C_{(γ)}H₂), 2.25 (m, 2H, Pal-C_{(α)}H₂), 2.52 (t, 2H, J=7.1 Hz, Lys-C_{(ε)}—H₂), 4.1-4.3 (m, 2H, Lys-C_{(α)}H, Ile-C_{(α)}H), 4.4-4.5 (m, 2H, 2× Glu-C_{(α)}H), 4.60-4.67 (m, 2H, 2× Glu-C_{(α)}H), 4.71-4.86 (m, 12H, 3× Gly-C_{(α)}H₂, 3×Ala-C_{(α)}H, 3× Val-C_{(α)}H).

IKVAV PA3: ¹H-NMR (600 MHz, TFA-d₁): δ 0.93 (t, 3H, J=6.8 Hz, Pal-CH₃), 0.99 (t, 3H, J=7.1 Hz, Ile-C_{(δ)}H₃), 1.04-1.16 (m, 18H, 3× Val-C_{(γ)}H₃, 3H, Ile-C_{(β)}H₃), 1.25-1.32 (m, 24H, several Pal-CH₂), 1.51 (m, 6H, 2×Ala-C_{(β)}H₃) 1.59-1.63 (m, 8H, 4× Glu-C_{(β)}H₂), 1.80 (dq, J=12.9, 1H, Val-C_{(β)}H), 2.25 (m, 2H, Pal-C_{(α)}H₂, 4H, 2× Glu-C_{(β)}H₂), 2.39 (m, 4H, 2×Glu-C_{(β)}H₂), 2.66 (t, 2H, J=7.1 Hz, Lys-C_{(ε)}—H₂), 3.86-4.01 (m, 5H, Ile-C_{(α)}H, 2× Glu-C_{(α)}H, Lys-C_{(α)}H, Val-C_{(α)}H), 4.55-4.62 (m, 2H, 2× Glu-C_{(α)}H), 4.77-4.82 (m, 6H, 3× Gly-C_{(α)}H₂), 4.86-4.92 (m, 3H, 2×Ala-C_{(α)}H, Val-C_{(α)}H), 4.98-5.01 (m, 1H, Val-C_{(α)}H).

IKVAV PA4: ¹H-NMR (600 MHz, TFA-d₁): δ 0.80 (t, 3H, J=6.9 Hz, Pal-CH₃), 0.86 (t, 3H, J=7.2 Hz, Ile-C_{(δ)}H₃), 0.91-1.06 (m, 18H, 3× Val-C_{(γ)}H₃, 3H, Ile-C_{(β)}H₃), 1.21-1.28 (m 24H, several Pal-CH₂), 1.45-1.51 (m, 8H, 4× Glu-C_{(β)}H₂), 1.56 (m, 6H, 2× Ala-C_{(β)}H₃) 1.71 (dq, J=12.9, 1H, Val-C_{(β)}H), 1.83 (m, 2H, Pal-C_{(α)}H₂, 4H, 2× Glu-C_{(β)}H₂), 2.13 (m, 4H, 2× Glu-C_{(γ)}H₂), 2.7 (t, 2H, J=7.3 Hz, Lys-C_{(ε)}—H₂), 4.14-4.22 (m, 5H, Ile-C_{(α)}H, 2× Glu-C_{(α)}H, Lys-C_{(α)}H, Val-C_{(α)}H), 4.24-4.29 (m, 2H, 2× Glu-C_{(α)}H), 4.43-4.52 (m, 6H, 3× Gly-C_{(α)}H₂), 4.62-4.68 (m, 3H, 2×Ala-C_{(α)}H, Val-C_{(α)}H), 4.73-4.86 (m, 1H, Val-C_{(α)}H).

IKVAV PA5: ¹H-NMR (600 MHz, TFA-d₁): δ 0.78 (t, 3H, J=6.8 Hz, Pal-CH₃), 0.84 (t, 3H, J=7.3 Hz, Ile-C_{(δ)}H₃), 0.90-0.94 (m, 12H, 2× Val-C_{(γ)}H₃, 3H, Ile-C_{(β)}H₃), 1.19-1.29 (m 24H, several Pal-CH₂), 1.44-1.49 (m, 11H, 4×Glu-C_{(β)}H₂, Ala-C_{(β)}H₃), 1.88 (m, 2H, Pal-C_{(α)}H₂), 2.11 (m, 8H, 4× Glu-C_{(γ)}H₂), 2.54 (t, 2H, J=6.9 Hz, Lys-C_{(ε)}—H₂), 4.13-4.29 (m, 5H, Ile-C_{(α)}H, 2×Glu-C_{(α)}H, Lys-C_{(α)}H, Val-C_{(α)}H), 4.41-4.47 (m, 2H, 2× Glu-C_{(α)}H), 4.62-4.67 (m, 8H, 4× Gly-C_{(α)}H₂), 4.72-4.79 (m, 2H, Ala-C_{(α)}H, Gly-C_{(α)}H₂), 4.73-4.86 (m, 1H, Val-C_{(α)}H).

IKVAV PA6: ¹H-NMR (600 MHz, TFA-d₁): δ 0.82 (t, 3H, J=6.9 Hz, Pal-CH₃), 0.88 (t, 3H, J=7.2 Hz, Ile-C_{(δ)}H₃), 0.93-1.06 (m, 18H, 3× Val-C_{(γ)}H₃, 3H, Ile-C_{(β)}H₃), 1.23-1.21 (m, 24H, several Pal-CH₂), 1.46-1.51 (m, 20H, 4× Glu-C_{(β)}H₂, 4× Ala-C_{(β)}H₃), 1.79-1.93 (m, 2H, Pal-C^{(γ)}H₂, 8H, 4× Glu-C_{(γ)}H₂), 2.09-2.20 (m, 8H, 4× Glu-C_{(γ)}H₂), 2.62 (t, 2H, J=6.9 Hz, Lys-C_{(α)}—H₂), 4.17 (d, 1H, J=17 Hz Ile-C_{(α)}H₂), 4.21 (d, 1H, J=17 Hz, Ile-C_{(β)}H₂), 4.45-4.49 (m, 5H, 4×Glu-C_{(α)}H₂, Val-C_{(α)}H), 4.55 (d, J=7.33 Hz, 1H, Val-C_{(α)}H), 4.61-4.71 (m, 4H, Ala-C_{(α)}H), 4.79-4.82 (m, 2H, Val-C_{(α)}H, Lys-C_{(α)}H), 4.87 (m, 1H, Val-C_{(α)}H).

IKVAV PA7: ¹H-NMR (600 MHz, TFA-d₁): δ 0.84 (t, 3H, J=7.1 Hz, Pal-CH₃), 0.89 (t, 3H, J=7.1 Hz, Ile-C_{(δ)}H₃), 0.89-1.03 (m, 18H, 3× Val-C_{(γ)}H₃, 3H, Ile-C_{(β)}H₃), 1.17-1.24 (m, 24H, several Pal-CH₂), 1.38-1.54 (m, 20H, 4× Glu-C_{(β)}H₂, 4× Ala-C_{(β)}H₃), 1.84-1.91 (m, 2H, Pal-C_{(α)}H₂, 8H, 4× Glu-C_{(γ)}H₂), 2.01-2.11 (m, 8H, 4× Glu-C_{(γ)}H₂), 2.72 (t, 2H, J=6.9 Hz, Lys-C_{(α)}—H₂), 4.14 (d, 1H, J=16.7 Hz Ile-C_{(α)}H₂), 4.21 (d, 1H, J=16.7 Hz, Ile-C_{(β)}H₂), 4.29-4.35 (m, 5H, 4×Glu-C_{(α)}H₂, Val-C_{(α)}H), 4.51 (d, J=7.12 Hz, 1H, Val-C_{(α)}H), 4.67-4.73 (m, 4H, Ala-C_{(α)}H), 4.72-4.81 (m, 2H, Val-C_{(α)}H, Lys-C_{(α)}H), 4.88 (m, 1H, Val-C_{(α)}H).

IKVAV PA8: ¹H-NMR (600 MHz, TFA-d₁): δ 0.85 (t, 3H, J=7.2 Hz, Pal-CH₃), 0.86 (t, 3H, J=7.1 Hz, Ile-C_{(δ)}H₃), 0.91-1.06 (m, 12H, 2× Val-C_{(γ)}H₃, 3H, Ile-C_{(β)}H₃), 1.11-1.20 (m, 24H, several Pal-CH₂), 1.29-1.44 (m, 20H, 4× Glu-C_{(β)}H₂, 4×Ala-C_{(β)}H₃), 1.81-1.92 (m, 2H, Pal-C_{(α)}H₂, 8H, 4× Glu-C_{(γ)}H₂), 2.02-2.14 (m, 8H, 4× Glu-C_{(γ)}H₂), 2.65 (t, 2H, J=7.1 Hz, Lys-C_{(ε)}—H₂), 4.09 (d, 1H, J=17.1 Hz Ile-C_{(α)}H₂), 4.13 (d, 1H, J=17.1 Hz, Ile-C_{(β)}H₂), 4.21-4.30 (m, 4H, 4×Glu-C_{(α)}H₂), 4.54-4.62 (m, 5H, Ala-C_{(α)}H), 4.69-4.84 (m, 2H, Val-C_{(α)}H, Lys-C_{(α)}H), 4.86-4.91 (m, 1H, Val-C_{(α)}H).

### Diffusion-Ordered Spectroscopy (DOSY)

PA materials were mixed (90 mol % of IKVAV PA2+10 mol % FGF2 PAs) and sonicated as described above and then lyophilized. Samples were then dissolved in D₂O water and solubilized in 1 equiv. NaOD at a concentration of 6 mM in standard 5 mm NMR tube with 0.25 mM sodium trimethylsilylpropanesulfonate (DSS) as a chemical shift reference and an intensity standard. After sonication for 20 min, the samples were annealed at 80° C. for 30 min, followed by slow cooling at room temperature. Diffusion coefficients were measured using pulse-field gradient NMR using the stimulated echo pules sequence with a 2 ms gradient pulse and a 100 ms diffusion delay time using a maximum gradient strength of 53 G/cm.

### 1. Simulation Procedures

The PAs were created in Avogadro and transformed to MARTINI force field coarse-grained (CG) representation using a modified version of martinize.py to include the palmitoyl tail and using coiled coil secondary structure for the peptide. The last two E residues (furthest from aliphatic tail) as well as the K and R residues in the epitopes were charged, while the first two E residues were treated as neutral as this was found to be ideal for fiber formation in previous simulations. The final charge is −1 for the IKVAV PAs and +3 for FGF2 PAs. Simulations were made in two steps in a cubic box 21.5×21.5×21.5 nm³ solvated with CG water and with enough ions to neutralize the systems. Firstly, 300 IKVAV PAs were randomly disposed on the box with a minimum intermolecular space of 3 Å. This gives a concentration of 50 mM (7.3-7.8 wt % for IKVAV PAs). This is within the range of concentrations commonly used to accelerate self-assembly simulations, which can be up to 10 times higher than the experimental system used. These systems were equilibrated for 10 s (FIG. 8). The fibers formed were centered in the box and 33 FGF2 PAs (10 mol %) were added randomly around the fibers with a minimum allowance of 3 Å. These systems were then equilibrated for 10 s in five independent simulations per system.

All visualizations were rendered using visual molecular dynamics (VMD). Coarse-grained molecular dynamic (CG-MD) simulations were performed in GROMACS 5.0.4, which was also used for the analysis of the simulations. A cut-off of 1.1 nm was used for intermolecular interactions using reaction field with a relative dielectric constant of 15 for electrostatics and potential-shift for Lennard-Jones interactions. All systems were minimized to 5000 steps or until the forces between atoms converged below 2000 pN. Self-assembly simulations were run using a 25-fs time step in NPT ensemble using V-rescale algorithm for the temperature (303 K, τT=1 ps) and Berendsen for the pressure (1 bar, τP=3 ps). Simulations were run for 100,000,000 steps corresponding to 10 s effective time.

Clustering was measured by considering a cutoff distance of 0.6 nm for PAs in the same cluster (FIG. 6J). Dynamism was measured using the root mean square fluctuations (RMSF) through the last 5 s of five simulations using the systems that are equilibrated after 5 s as shown by the root mean square fluctuations, RMSD. Therefore, excluding for this analysis the systems were some FGF2 PAs were not attached to the fibers before the last 5 s as they had a masking effect in the results. For visualization, the RMSFs were converted to 0-factors and all the results normalized to get comparable color codes.

### 2. In Vitro Cell Culture

### Human Umbilical Vein Cord (HUVEC) Culture

Human umbilical vein cord (HUVECs) (pooled donor, LONZA, Allendale, New Jersey) were grown to 70-80% confluence for each experiment (P2-P4) in a T-75 cell culture flask using complete media (Endo GRO-VEGF Complete Culture Media Kit, Millipore) supplemented with 1% penicillin-streptomycin. Media was changed every 3 days.

### HUVEC Treatments and Coatings

Treatments were prepared by dissolving the co-assembled PAs in media without serum. The total concentration of FGF2 PAs per treatment was 0.5 μM. FGF-2 native protein (Peprotech) was resuspended and used at 0.25 nM. For western blot experiments, HUVEC were cultured in 12 well plates at a density of approximately 150,000 cells/well for 2 days in vitro before being treated. Treatments were added for 2 h-2 days in vitro before cell lysates were harvested or cells were fixed. For PA coatings, PAs were painted on the coverslips (German Glass, Chemglass Life Science) or tissue culture plates as described in the Co-assembled PA preparation section. Three replicates per condition and three independent experiments were carried out for all conditions investigated.

Human Neural Progenitor Cell (hNPCs) Culture

Neural progenitor cells (NPCs) were differentiated from human embryonic stem cells HUES 64 (Harvard University) (see FIG. 2C). In short, 70% confluent stem cell cultures grown in Matrigel (Thermo Fisher) coated plates with mTESR medium (STEMCELL Technologies), were switched to N2B27 medium (50% DMEM: F12, 50% Neurobasal, supplemented with NEAA, Glutamax, N2 and B27; Gibco) containing dual SMAD inhibitors (SB431542, DNSK International and LDN-193189, Tocris) for 12 days to induce the generation of neural progenitors. Neuralization was enhanced by supplementing medium with laminin from day 5 to 12. Next, neural rosettes were dissociated with neural rosette selection reagent (STEMCELL Technologies) to obtain NPCs, which are expanded in N2B27 (Gibco) medium supplemented with bFGF (Millipore). For experimentation in the different PA platforms, NPCs were dissociated with Accutase (Innovative Cell technology), and cultured in the distinct platforms with DMEM: F12+N2+B27 medium with hyclone penicillin-streptomycin (GE Healthcare) and ascorbic acid (0.2 μg/mL; Sigma-Aldrich).

Human Neural Progenitor Cell (hNPCs) Cultures Treated with IKVAV PAs or Seeded on Co-Assembled PA Coatings

For IKVAV PA treatments, hNPCs were cultured on ornithine coatings (German Glass, Chemglass Life Science) in 6 well and 24 well plates at a density of 500,000 cells/well and 80,000 cells/well respectively. hNPCs were treated with 60 mM of IKVAV PAs ([PA]=6 mM, [KCl]=3 mM, [NaCl]=150 mM) or recombinant laminin protein (10 mg/mL) in solution. IKVAV PA2 and IKVAV PA5 were also mixed with 5 mM CaCl₂) at a ratio PA:CaCl₂) 6:1 to treat hNPCs. For 2D experiments, hNPCs were cultured on the different IKVAV PAs or laminin coatings in 6 well and 24 well plates at densities mentioned above. For co-assembled experiments, hNPCs were cultured on the different co-assembled PA coatings in 6 well and 24 well plates at a density of 400,000 cells/well and 50,000 cells/well respectively.

hNPCs were fed 4 times a week with DMEM: F12+N2⁺B27 medium with hyclone penicillin-streptomycin and ascorbic acid. After 24 h, 72 h 1- or 2-weeks in vitro, cell lysates were harvested or fixed for WB or ICC, respectively. Three replicates per condition and at least three independent experiments were carried out for all conditions investigated.

### Surface Sensing of Translation (SUnSET)

hNPCs cultured with the different IKVAV PA treatments were pulsed for 10 min with puromycin (20 μM, Sigma-Aldrich) at 37° C. Cells were pre-treated 2 h before puromycin pulse with Cycloheximide (100 mg/mL, Calbiochem, Millipore) as a translation inhibitor control. 30 g protein extracts were obtained and loaded on Mini-PROTEAN TGX Stain-Free gels (4 to 20% gradient, BIO-RAD). Total protein signal was detected with ChemiDoc™ XRS+(Bio-Rad) and newly synthesized protein was detected by western blot with anti-puromycin antibody (mouse anti-puromycin 1:5000, Millipore).

### Cell Viability Assay

HUVEC and hNPCs were treated or cultured on the different co-assembled PA systems for 2 days-2 weeks in vitro. Media was removed, and cells were rinsed once with HBSS 1× (Gibco). A Calcein-AM/ethidium homodimer-1 live/dead assay (Invitrogen) was used to assess cell viability. Calcein-AM/ethidium homodimer-1 solution in HBSS was added to each well for 20 min at room temperature (RT). The solution was removed, and samples were rinsed 3 times with HBSS (Gibco) before coverslips were mounted for imaging.

### EdU Analysis

For the hNPCs proliferation assay, 2 μM of the thymidine analog 5-ethynyl-2′-deoxyuridine (EdU, Thermo Fisher) was incorporated into the culture medium for 24 h. Cells were fixed at the indicated time points in 4% paraformaldehyde (PFA) for 15 min at RT. After fixation, samples were washed in PBS twice and then stained with Click-iT™ EdU Cell Proliferation Kit containing 11 mM CuSO4 (from 50 mM stock) and 1 μg/mL of Alexa Fluor-555 azide (from 0.5 mg/mL, Thermo Fisher) for 30 minutes at RT in the dark. The staining cocktail was then removed, and cells were washed with PBS. Then, hNPCs were counterstained with anti-rabbit SOX-2 antibody following the immunofluorescence protocol described below and 4′, 6-diamidino-2-phenylindole (DAPI, Thermo Fisher) at 5 μg/mL in PBS for 20 min at RT. The cells were then washed with PBS and mounted with Immu-Mount (Fisher Scientific) for imaging.

### 4. In Vivo Studies:

All animal housing and procedures were performed in accordance with the Public Health Service Policy on Humane Care and Use of Laboratory Animals. All procedures were approved by the Northwestern University Institutional Animal Care and Use Committee.

### Surgical Procedure

All experiments were conducted at the Simpson Querrey Institute at Northwestern University. 182 CD1 mice (Charles River) were subjects of this study (N=38 Sham, N=38 IKVAV PA2 alone, N=38 IKVAV PA2+FGF2 PA1, N=38 IKVAV PA2+FGF2 PA2, N=12 IKVAV PA1, N=12 Backbone PA, N=6 IKVAV PA4). The age of animals used in the study was in the range of 10-16 weeks. 8 independent in vivo experiments were carried out (injury+PA injection) and each of the 8 experiments had animals of the exact same age which were injected with at least the 4 main treatment groups: Sham, IKVAV PA2 alone, IKVAV PA2+FGF2 PA1, and IKVAV PA2+FGF2 PA2. Animals were anesthetized using 2.5% isoflurane gas with oxygen. A laminectomy was performed to expose the spinal cord at the T10-T11 spinal level. A severe contusive injury was performed using the Infinite Horizon Spinal Cord Impactor system (IH-0400 Precision Systems and Instrumentation) with 85 kdyn of impact force and a dwell time of 60 s. After the lesion, the skin was sutured using 9 mm wound clips (BD Biosciences) and the animals were recovered on a heating pad to maintain body temperature. Bladders were manually expressed daily during the entire 12 weeks of the experiment.

### Animal Inclusion and Exclusion Criteria

All mice were evaluated in an open-field environment 24 h after the lesion and animals exhibiting any hindlimb movements (score higher than 0 in the BMS score) were discarded from the study. Mice that passed this inclusion criterion were randomized into experimental groups for PA injection and were thereafter evaluated blind to their experimental condition.

### Injection Procedure

PAs (dissolved in sterile NaCl 0.9% solution at 1 wt %) were injected 24 h after SCI using a glass capillary micropipette (Sutter Instruments, Novato, CA) (outer diameter, 100 m) coated with Sigmacote (Sigma-Aldrich) to reduce surface tension as described elsewhere (19). The capillaries were loaded onto a Hamilton syringe using a female Luer adaptor (World Precision Instruments) controlled by a Micro4 microsyringe pump controller (World Precision Instruments). Under isoflurane anesthesia, autoclips were removed and the injury site was exposed. At 24 h post injury, the laminectomy in the spinal column was still intact and the bruise created by the lesion was visible. A stereotaxic Kopf apparatus was used to position the micropipette just dorsal to the lesion. The micropipette was lowered to a depth of 750 m measured from the dorsal surface of the cord, and 4-6 μL of the diluted amphiphile solution was injected at 1 L/min. The micropipette was withdrawn at intervals of 250 m to leave a trail (ventral to dorsal) of PA within the cord. At the end of the injection, the pipette was left in place for 2-3 additional minute to allow material gelation, after which it was withdrawn, and the wound was closed with 9 mm wound clips.

### Hindlimb Locomotor Evaluation

The motor function was evaluated with the locomotor open-field rating scale on the Basso Mouse Scale (BMS). A team of two experienced examiners evaluated each animal for 5 to 10 minutes and assigned a defined score for each hindlimb. For the footprint analysis, the hindlimbs of the mice were dipped in colored dyes. A narrow runway (80 cm length and 4 cm width) was lined with white paper as the animal walked across. The stride length was defined as the distance from the start to the end of a step with the back paw. The stride width was defined as the distance from the left outermost toe to the right outermost toe. All measurements were taken on each side for three consecutive steps and were averaged.

### Anterograde BDA Corticospinal Tracing

14 days before perfusion, 6 animals were used per condition to trace the corticospinal tract. The animals were anesthetized using 2.5% isoflurane gas in oxygen. The head of each mouse was stabilized using the stereotaxic frame (Stoelting Co.). An 8 mm incision was made through the skin along the midline of the skull using a surgical scalpel blade. Injections of 0.25 L of 10% biotinylated dextran amines (BDA, molecular weight=10,000, Thermo Fisher Scientific) were placed into each hemisphere spanning the motor cortex. The following coordinates were used: from bregma, rostral-caudal: ±0.0, 0.5, 1.0 mm; lateral: ±1.0, 1.5 mm; depth: 0.7 mm. Sections were processed for BDA with streptavidin-conjugated secondary antibody 555 (Thermofisher).

### BrdU Injections

24 h after PAs were injected into the spinal cords, 6 animals were used per condition to inject 5-bromo-2′-deoxyuridine (BrdU, Sigma-Aldrich) intraperitoneally (5 mg/10 g body weight) for a 7-day period (1 pulse per day). BrdU incorporation was analyzed by immunohistochemistry (rat-anti-BrdU, 1:1000; Abcam) at 12 weeks after PA injection.

### DiI Labelling

After deep anesthesia and shortly before perfusion, 20 μL/mL of DiI (Sigma-Aldrich) in PBS solution with 5 w/v % of glucose was transcardially injected into 6 animals per condition, as previously described (21). Immediately after, animals were transcardially perfused using an isotonic solution containing 4% of paraformaldehyde (PFA, in 0.4 M phosphate buffer, pH 7.4)

### Animal Sacrifice and Tissue Processing

All animals were sacrificed using an overdose of carbon dioxide and transcardially perfused with phosphate buffered saline (PBS) followed by 4% paraformaldehyde (PFA, Sigma-Aldrich) in isotonic solution (0.4 M phosphate buffer, pH 7.4). The spinal cords were fixed for 4-6 h in 4% PFA and overnight in PBS containing 30% of sucrose (Sigma-Aldrich). The spinal cords were then frozen in PBS containing 30% sucrose (Sigma-Aldrich) and 15% gelatin (Sigma-Aldrich) and sectioned on a Leica CM1850 cryostat at 40 m thick.

### 5. Biological Assays for In Vitro and In Vivo

### Immunofluorescence

For immunofluorescence, fixed primary cultures or free-floating tissue sections (40 m Thick) were incubated in blocking buffer containing 0.02% Triton-X (Sigma-Aldrich), 1% NHS (Gibco) and PBS 1× (Gibco). Samples were incubated with primary antibodies over night at 4° C. Alexa-488, Alexa-555 and/or Alexa-647 (1:500, Thermofisher) were used as secondary antibodies. DAPI (1:500, Thermofisher) was used to stain nuclei. Finally, the samples were mounted on coverslips with Immu-Mount (Fisher Scientific) solution for imaging.

### Western Blot (WB)

RIPA buffer (Thermofisher) with a cocktail of Halt Protease and Phosphatase Inhibitors (Thermofisher) was used to extract protein from in vitro and in vivo samples. For in vivo studies, spinal cord tissue was then sonicated using a horn sonicator (Branson) to break up the tissue. A Pierce™ BCA Protein Assay Kit (Thermofisher) was used to determine protein content for all samples used. Protein extracts obtained from cell cultures or tissue were separated using an SDS-polyacrylamide gel and electro-transferred to a nitrocellulose membrane (Bio-Rad). Membranes were blocked for 30 minutes to 1 hour using a 5% milk solution (Bio-Rad) and incubated over night with primary antibodies. Corresponding secondary HRP-conjugated antibodies (1:1000, ThermoFisher) were used for 1 h at room temperature. Radiance Bioluminescent ECL substrate (Azure Biosystems) was used to detect protein signal. The membranes were imaged using the Azure Biosystems imager and densitometry analysis were performed using ImageJ software.

### Antibodies for Immunofluorescence and Western Blot

The following primary antibodies were used for in vitro and/or in vivo studies: rabbit anti-GFAP (1:1000, Dako, Z0334), rabbit anti-laminin B1 (1:1000, Sigma-Aldrich), rabbit anti-Actin (1:2000, Sigma-Aldrich, A2066), mouse anti-GAPDH (1:2000, Cell Signaling, 97166), goat anti-5HT (1:1000, Abcam, mab66047), rabbit anti-CD31 (1:100, BD Pharmigen, 550274) mouse anti-Neurofilament (NF, 1:2000, Millipore, MAB1592), rabbit anti-GAP43 (1:2000, Cell Signaling, 8945), goat anti-Sox-2 (1:1000, Abcam, ab110145), Streptavidin Alexa Fluor™ 555 Conjugate (1:500, Thermofisher, S32355), rabbit anti-PH3 (1:1000, Cell Signaling, 9701), rat anti-BrdU (1:1000, Abcam, ab6326), rabbit-anti-MBP (1:500, Abcam, ab40390), mouse anti-NeuN (1:1000, Millipore, MAB377), goat anti-ChAT (1:1000, Millipore, AB144P), rabbit anti-FGFR1 (1:500, Cell signalling, 3472), p-FGFR1 (1:500, Cell signalling, 3471), mouse anti-ITGB1 (1:250, Millipore, clone HUTS-4, MAB2079Z), rabbit anti-p-ERK1/2 (1:1000, Abcam, ab196883), TUJ-1 (1:2000, Biolegend, 802001 and 801213), mouse anti-Pax-6 (1:500, Millipore, AD2.38), rabbit anti-Ki67 (1:500, Abcam, ab15580), rabbit anti-Nestin (1:1000 Millipore, ABD69) rabbit anti-ILK (1:500, Cell Signaling, 3862), rabbit anti-p-FAK (1:1000, Cell Signalling, 3281S), rabbit anti-FAK (1:1000, Cell Signaling, 3285S).

### Blood Vessel Analysis

To assess in vitro tubulogenesis and in vivo angiogenesis, an ImageJ (Fiji) script was established to automatically calculate 1) area fraction of blood vessels, 2) blood vessels length, and 3) number of branches. Images were processed, binarized and analyzed. Newly generated blood vessels in vivo were identified by quantifying the amount of CD31/BrdU double-positive cells within the region of interest. Functional blood vessels were identified using DiI staining in 8 sections within the lesion per mouse. 6 animals per group treatment were used in this analysis. The quantified cross sections were chosen as the first serial cross sections within the lesion that had DiI staining.

### Neurite Tracking

Axons labeled using BDA (Thermofisher, N7167) or stained with 5HT (Abcam) were quantified using Imaris® software version 9.3 as previously described (21). Lines were drawn across longitudinal spinal cord sections from the proximal border (PB) to the distal border (DB) of the SCI lesion at consistent distances and the number of axons intercepting the lines drawn was counted by researchers that were blinded to the experimental conditions. Multiple sections through the middle of the cord, where BDA or 5HT staining was denser, were counted per mouse and expressed as total intercepts per location, per animal. 6 animals per group treatment were used in these analyses.

### Degradation Studies and Tissue Clearing

Degradation studies of the PAs injected into the lesioned spinal cord were performed by covalently labeling the IKVAV sequence with Alexa-647 (click) fluorescent dye. At different time points after PA injection (2 weeks, 4 weeks, 6 weeks and 12 weeks) spinal cords were perfused, extracted, and cleared using benzyl alcohol-benzyl benzoate (BABB, Sigma-Aldrich). After clearing, spinal cord tissue became fluorescent at 488 nm. Full reconstructions were performed using spinning disk confocal microscopy and analyzed using Imaris software. Three spinal cords per group treatment and time point were used in these studies.

### Imaging

A Nikon AIR confocal laser-scanning microscope with GaAsP detectors, Nikon W1 Dual Cam Spinning Disk Confocal and Nikon A1RMP+ Multiphoton were used to visualize and image fluorescent cells, sections or full cleared spinal cord samples. Nikon Ti2 Widefield was used to acquire larger sections of spinal cords.

### Image Quantification and Analysis

For in vitro cell quantification, image files were imported into NIH Image J (1.51) software and the “analyze particles” and “cell counter” functions were used to measure the total number of cells in a determined area. Serial tissue sections were stained with NeuN and ChAT using free-floating immunostaining were quantified using Nikon Elements software. The rostral and caudal borders of the lesions were chosen as the first cross-sections that had NeuN staining in all four gray matter horns. 8 sections within the lesion were chosen per animal and expressed as number of neurons per section. Automated multichannel image acquisition, image stitching, and z-stack reconstruction (36-40 mm thick) were carried out on a Nikon GasP R1 confocal microscope to image the entire selected cross-section for NeuN and Chat markers for all conditions.

Fluorescence intensity of tissue sections stained for GFAP was analyzed using NIH Fiji Software. Scanned images with constant exposure settings in the various microscopes mentioned above were used for this analysis. Single channel immunofluorescence images were used to analyze the number of fluorescent positive pixels along the area selected in each image. Using “Plot Profile” function in ImageJ software, the average pixel values in reference to position along the line drawn were obtained. In order to plot the GFAP vs. distance, five sections through the middle of the cord were counted per mouse. 6 mice per group treatment were used for image quantification.

### Statistical Analysis

Data was analyzed using GraphPad Prism software (version 9.12). For each statistical analysis, for a quick identification of a normal distribution, we used Q-Q plots or frequency distribution plots (histograms). We also tested whether sample data fit into a Gaussian distribution using the Shapiro Wilk test and D'Agostino-Pearson omnibus normality tests. Comparisons between pairs of experimental groups were performed using unpaired Student's t-test. Comparisons among three or more groups were conducted using one-way ANOVA with post hoc independent pair-wise analysis as per Bonferroni. Mouse BMS behavioral scores were compared between PA- and saline-treated or between PA groups by two-way repeated measures ANOVA with Bonferroni. The statistical tests and parameters including the definitions and the number of experiments is reported in the corresponding figure legends. The in vitro data were represented using bar graphs. The error bars represent 30 images per experiment and 3 independent experiments per condition. The in vivo data were represented using dot plots, where each data point represents the value for one in vivo animal or tissue section (for all the staining experiments the ICC was repeated independently with tissue from 6 different animals with similar results. 8 images 36-40 mm thick were taken per animal per group). All data were presented as mean±standard error of the mean (SEM) unless otherwise noted.

1. A supramolecular assembly comprising at least two peptide amphiphiles, wherein the at least two peptide amphiphiles comprise:
a. at least one IKVAV peptide amphiphile comprising a hydrophobic segment, a structural peptide segment, a charged peptide segment, and a bioactive peptide comprising the amino acid sequence IKVAV (SEQ ID NO: 1); and
b. at least one growth factor mimetic peptide amphiphile.

2. The supramolecular assembly of claim 1, wherein the at least one IKVAV peptide amphiphile comprises a fluorescence anisotropy value of less than 0.3.

3. The supramolecular assembly of claim 1, wherein the at least one IKVAV peptide amphiphile comprises a proton relaxation rate (¹H—R₂) of less than 4s⁻¹.

4. The supramolecular assembly claim 1, wherein the hydrophobic segment comprises an 8-24 carbon alkyl chain (C₈₋₂₄)₂ the structural peptide segment comprises a sequence having at least 50% identity to A₂G₂ (SEQ ID NO: 4), and/or the charged peptide segment comprises a sequence having at least 50% identity to E₂, E₃, or E₄ (SEQ ID NO: 11).

5. (canceled)

6. (canceled)

7. The supramolecular assembly of claim 4, wherein the structural peptide segment comprises A₂G₂ (SEQ ID NO: 4) and the charged peptide segment comprises E₂, E₃, or E₄ (SEQ ID NO: 11).

8. The supramolecular assembly of claim 1, wherein the bioactive peptide is attached to the charged peptide segment by a linker.

9. The supramolecular assembly of claim 8, wherein the linker is a peptide linker or a non-peptide linker.

10. The supramolecular assembly of claim 1, wherein the at least one IKVAV peptide amphiphile comprises
(SEQ ID NO: 12)
C₈₋₂₄-A₂G₂E₄GIKVAV.

11. The supramolecular assembly of claim 1, wherein the at least one growth factor mimetic peptide amphiphile comprises a hydrophobic segment comprising an 8-24 carbon alkyl chain (C₈₋₂₄), a structural peptide segment comprising a sequence having at least 50% identity to V₂A₂ (SEQ ID NO: 3) or A₂G₂(SEQ ID NO: 4), a charged peptide segment comprising a sequence having at least 50% identity to E₂, E₃, or E₄ (SEQ ID NO: 11), and a growth factor mimetic peptide sequence.

12. The supramolecular assembly of claim 11, wherein the growth factor mimetic sequence is a vascular endothelial growth factor (VEGF) mimetic sequence, a fibroblast growth factor 2 (FGF-2) mimetic sequence, a Glial cell-derived neurotrophic factor (GDNF) mimetic sequence, a brain-derived neurotrophic factor (BDNF) mimetic sequence, or a Netrin-1 mimetic sequence.

13. (canceled)

14. The supramolecular assembly of claim 11, wherein the growth factor mimetic sequence is an FGF-2 mimetic sequence having at least 70% sequence identity to YRSRKYSSWYVALKR (SEQ ID NO: 2).

15. The supramolecular assembly of claim 11, wherein the growth factor mimetic sequence is attached to the charged peptide segment by a linker.

16. The supramolecular assembly of claim 15, wherein the linker is a peptide linker or a non-peptide linker.

17. The supramolecular assembly of claim 11, wherein the at least one growth factor mimetic peptide amphiphile comprises
(SEQ ID NO: 13)
C₈₋₂₄-V₂A₂E₄GYRSRKYSSWYVALKR
or
(SEQ ID NO: 14)
A₂G₂E₄GYRSRKYSSWYVALKR.

18. The supramolecular assembly of claim 11, wherein the at least one IKVAV peptide amphiphile comprises
(SEQ ID NO: 12)
C₈₋₂₄-A₂G₂E₄GIKVAV
and the at least one growth factor mimetic peptide amphiphile comprises
(SEQ ID NO: 13)
C₈₋₂₄-V₂A₂E₄GYRSRKYSSWYVALKR
or
(SEQ ID NO: 14)
A₂G₂E₄GYRSRKYSSWYVALKR.

19. The supramolecular assembly of claim 18, wherein the at least one IKVAV peptide amphiphile comprises
(SEQ ID NO: 12)
C₈₋₂₄-A₂G₂E₄GIKVAV
and the at least one growth factor mimetic peptide amphiphile comprises
(SEQ ID NO: 13)
C₈₋₂₄-V₂A₂E₄GYRSRKYSSWYVALKR.

20. A composition comprising the supramolecular assembly of claim 1.

21.-42. (canceled)

43. A method of treating a nervous system injury in a subject, comprising providing to the subject the composition of claim 20.

44. The method of claim 43, wherein the nervous system injury is a central nervous system injury.

45. The method of claim 44, wherein the central nervous system injury is a spinal cord injury.

46. The composition of claim 1, wherein the structural peptide segment has a propensity for forming β-sheet conformations.

47. The composition of claim 1, wherein the structural peptide segment has a total propensity for forming β-sheet conformations of 4 or less.

48. The composition of claim 1, wherein the charged peptide segment is acidic, basic, or zwitterionic.

49. The composition of claim 1, wherein a molar ratio of the at least one IKVAV peptide amphiphile to the at least one growth factor mimetic peptide amphiphile in the composition is about 80:20 to about 99:1.
